(19) United States
(12) Patent Application Publication
(10) Pub. No.: US 2020/0350072 A1
(43) Pub. Date: Nov. 5, 2020

(54) DIAGNOSITIC AND TREATMETNT TOOL AND METHOD FOR ELECTRONIC RECORDING AND INDEXING PATIENT ENCOUNTERS FOR ALLOWING INSTANT SEARCH OF PATIENT HISTORY

(71) Applicant: MIRR LLC, Houston, TX (US)

(72) Inventors: David McEwing, Houston, TX (US); Omar Higgs, Los Angeles, CA (US)

(73) Assignee: MIRR LLC, Houston, TX (US)

(21) Appl. No.: 16/859,260

(22) Filed: Apr. 27, 2020

Related U.S. Application Data
(63) Continuation-in-part of application No. 16/532,152, filed on Aug. 5, 2019.
(60) Provisional application No. 62/942,827, filed on Dec. 3, 2019.
(60) Provisional application No. 62/942,841, filed on Dec. 3, 2019.
(60) Provisional application No. 62/923,676, filed on Oct. 21, 2019.
(60) Provisional application No. 62/728,756, filed on Sep. 8, 2018.
(60) Provisional application No. 62/750,300, filed on Oct. 25, 2018.
(60) Provisional application No. 62/714,845, filed on Aug. 6, 2018.

(51) Int. Cl. G16H 50/20; G16H 10/60; G16H 70/20; G16H 50/70; G06Q 30/02; G16H 70/60; G06F 16/25; A61B 5/00; A61B 5/08; G06F 40/247

(52) U.S. Cl. CPC G16H 50/20; G16H 10/60; G16H 70/20; G16H 50/70; G06Q 30/0283; G06F 40/247; G06F 16/252; A61B 5/4851; A61B 5/0816; A61B 5/4585; G16H 70/60

(57) ABSTRACT

Device and method for improved diagnosis and treatment of patients. Detailed coding protocols are instantly assigned to diagnoses and treatment plans created by clinicians based upon initial patient encounter. Past patient records (EHR) can be instantly searched using the assigned codes during the patient encounter, thereby allowing the clinician to make a more complete diagnosis and plan of treatment. The search of past patient records utilizes the assigned codes as a search index thereby filtering out unrelated-non relevant patient medical history. Clinician notations of symptoms and possible diagnosis can be utilized for an expanded correlation to codes of medical code protocols relevant to treatment or tests. This expanded listing of codes can be displayed to the clinician during the patient encounter for evaluation in determining a diagnosis. Correlated medical codes can be disclosed with calculated or past recorded reimbursement information.

### RELATED APPLICATIONS

This application is a CONTINUATION IN PART of pending application Ser. No. 16/532,152 filed Aug. 5, 2019 entitled “INDEXING OF MEDICAL RECORDS UTILIZING CODING PROTOCOLS”. This application claims priority to the Ser. No. 16/532,152 application which is incorporated by reference herein in its entirety.

The Ser. No. 16/532,152 application claims priority to provisional application Ser. No. 62/714,845, entitled “INDEXING OF MEDICAL RECORDS UTILIZING CODING PROTOCOLS” and filed Aug. 6, 2018, said application is incorporated herein by reference in its entirety. The Ser. No. 16/532,152 application also claims priority to provisional application Ser. No. 62/728,756, entitled “INDEXING OF MEDICAL RECORDS UTILIZING CODING PROTOCOLS”, filed Sep. 8, 2018 and said application is also incorporated herein by reference in its entirety. Further, the Ser. No. 16/532,152 application claims priority to provisional application Ser. No. 62/750,300 entitled “INDEXING OF MEDICAL RECORDS UTILIZING CODING PROTOCOLS” filed Oct. 25, 2018, said application being incorporated herein by reference in its entirety.

This application also claims priority to provisional applications 62/923,676 filed Oct. 21, 2019, entitled “Disclosure of Reimbursement with Suggested Code Display”, 62/942,827 filed Dec. 3, 2019, and entitled “Display or Search of Treatment and Test Codes Correlated to Treatment”, and 62/942,841 filed Dec. 3, 2019 entitled “Display or Search of Diagnosis, Treatment and Test Codes”. These three provisional applications are incorporated herein by reference in their entirety.

### FIELD OF USE

This disclosure pertains to electronic recording of clinician notes of patient encounters, thereby creating patient medical records (EMR) of a single office or facility or patient electronic health records (EHR) that are accessible from multiple facilities. The records are contemporaneously indexed utilizing one or more established and accepted alphanumeric code protocols wherein the protocols may be the same utilized for achieving reimbursement of medical services from third party payors. The codes are assigned to specific medical diagnoses, treatments or tests. The codes have been prepared by medical professionals and provide individual codes to detailed diagnoses, treatments and tests. This disclosure teaches incorporating the coding protocols into EMR or EHR records that therefore may be instantly searched utilizing the coding as an index. Instant access taught by this disclosure to relevant portions of a patient history facilitate accurate diagnosis and initiation of treatment and testing. It also facilitates understanding of patient history and medical prognosis in exigent circumstances wherein scarce medical resources must be allocated among competing needs. Protocols include the International Classification of Diseases (ICD), the Current Procedural Terminology (CPT) and the Healthcare Common Procedure Coding System (HCPCS) code protocols. This disclosure also pertains to conducting searches of patient specific electronic health records utilizing one or more indexing alphanumeric code protocols.

Also included in the disclosure is a system including a device for electronically entering patient identifying information, diagnostic information or symptoms, other medical information pertaining to tests or treatments and entering appropriate codes from a selected protocol correlated to the information. The device taught by this disclosure allows speedy entry of information by the clinician and instant display of possible relevant detailed codes that can be used for searching the patient's electronic records. The information can be transmitted electronically to a repository of EHR records or to the databases of individual medical facilities, collectively referred to as EHR records. The device may allow “one touch” initiation of searches across multiple repositories in multiple states. The device may allow initiation of the repository searching its records and transmitting the search results to the device or other display or storage mechanism. The electronic search results utilizing the coded index may be made available to the clinician in real time thereby facilitating appropriate patient treatment.

It will be appreciated that without access to a patient's medical history, the clinician may only treat the observed symptoms in contrast to the underlying problem or cause.

### Related Prior Art

A medical health care provider creates a record of patient diagnosis and treatment. The record is individualized by patient. The medical records are patient specific. These records may be hand written and unindexed (other than perhaps by chronological order).

It is difficult for a subsequent medical health care provider to rapidly obtain access to the patient's records of earlier test, treatments or diagnosis that were conducted by earlier and separate health care providers. Requests require the patient's written consent and this consent must be provided to the earlier health care provider before there can be any release of patient records.

A patient's records of earlier treatment, testing and diagnosis may be in a variety of locations. The patient may have a limited recollection of the prior medical health care providers and have no contact information.

Records that are received are often copies of paper records with multiple handwritten notations that may be difficult to timely decipher. Much of the paper records that are received may be unrelated to the existing medical event and therefore be of little or no value to the subsequent/requesting medical health care provider.

Also a patient's medical record may currently be stored or recorded on an electronic format. (The patient's electronic medical record, typically located at a single facility, is termed an EMR.) The records may be in a format that does not easily allow the EMR to be searched for particular topics. It may be necessary to attempt to convert some or all of the EMR files to an electronic format compatible to the format of the requesting medical health care provider. The requesting health care provider may be at a different health care facility. The EMR is in an electronic format and identifies the patient and the date that service was provided. The EMR is intended to be the electronic equivalent of the paper records. A collection of multiple patient specific EMRs from multiple entities is termed a patient's EHR.

The EMR records include the description of the patient, the symptoms, the diagnosis and treatment. The EMR records may be specific to each health care provider, e.g., physicians, laboratories, etc., or health care facility, e.g., clinic or hospital, etc. The records may be paper or recorded in one of multiple electronic media. As stated above, the media may be incompatible with the system or format utilized by the requesting entity (subsequent medical health care provider). The records are seldom, if ever, effectively indexed by date, medical event or tests/procedures.

All of these limitations make timely receipt of relevant past medical treatment and testing to be very problematic. Although the prior records may be retained in an electronic format, there are very significant issues of compatibility of formats and an inability to perform searches, e.g. word search of the records. According it is necessary to parse the received records similar to that required of paper records. The result is that the patient EMR records may, at best, be communicated only inside of an individual hospital or physician's office.

The above described current medical practice of maintained medical records, often relying on paper records or variously maintained EMR formats at disparate locations, can be voluminous. Accessing the past health care records is a time consuming matter and may not be effective in attempting to provide best care to the patient in real time. It will be appreciated that this problem may be especially acute in emergency care situations.

There has been increasing emphasis to maintain the patient health care records in an electronic format. This would shrink the storage burden of maintaining archives of voluminous patient paper files. It has the potential to also speed the retrieval of past information for the benefit of the current health care provider and patient. However there is not uniform electronic record storage format. There are multiple electronic medical record formats and service providers. Examples of EMR service providers include Cerner, Epic, and Allscripts.

There also has been considerable effort to document patient diagnosis, treatment and testing using a common protocol of descriptors. Several attempted comprehensive protocols have been developed to standardize patient histories. These standard protocols utilize alphanumeric coding schemes wherein different diagnoses have separate codes. Similarly, different treatment procedures have individualized alphanumeric codes. Also test and laboratory testing procedures are included in these coding protocols. There complexity of medicine and the tremendous variety of aliments and diseases has required these coding protocols to incorporate tens of thousands of separate codes.

This coding effort has been adopted by third party payors such as health insurance companies, Medicare and Medicaid, in an attempt to standardize reimbursement for the same or similar procedure, understanding that the multiple health care providers use different terminology to describe the same or comparable services. However due to the great number of codes that can be assigned and the vast number of procedures and diagnosis, it has been frequently necessary to create a class of employees, i.e., coders. to review medical records of treatment and assign the appropriate codes. Obtaining consistency and accuracy is a significant task. Typically, matching the clinician notes to appropriate coding takes place hours after the initial patient encounter. This prevents the coding to be a basis or index for real time record searching during a patient encounter as taught below by the disclosure. This disclosure, in addition to the utilization of these detailed codes as an instant search indexing mechanism available to the clinician, e.g., treating physician, provides code selection by the clinician at the time of a patient encounter in contrast to current practice, deciphering of clinician notes hours or days later and assigning codes based upon this decryption.

There is also the factor of facilitating the sharing of patient medical information while maintaining patient privacy. The Health Insurance Portability and Accountability Act of 1996 (“HIPAA”) imposes controls for privacy protection. This includes a requirement that the patient consent to any disclosure of information not intended for rendering medical services to the individual patient.

### BRIEF SUMMARY OF DISCLOSURE

Electronic health records (EHR) are understood to contain all of the information recorded in paper records and charts or contained in EMRs compiled at individual facilities. The EHRs are designed to collect all medical information from all healthcare sources, including multiple physicians, hospitals, and the patient themselves.

In addition to providing a record of patient care, including diagnoses, procedures/treatments and test results, the medical health care provider's records, either maintained in paper or electronic format, are used to generate invoices for submission to the patient or a third-party payor, typically an insurance provider. The records of the individual hospital or medical service provider may be termed “patient medical records” (PMR). If maintained electronically, the records may be designated EMR.

To facilitate payment of medical invoices by third party payors, i.e., typically health insurance companies, the medical services are converted or indexed to a uniform coding scheme or protocol (hereinafter “coding protocol”). There are differing systems. Medical service coding protocols subject of this disclosure include but are not limited to the International Classification of Diseases (ICD), the Current Procedural Terminology (CPT) and HCPCS protocols.

In current practice, the PMR created by the physician or other service provider may be reviewed by a medical billing coder. The medical billing coder (residing as an employee or contractor of the medical service provider) assigns alpha-numeric codes consistent with diagnosis, treatment, procedures or testing. The coding is in accordance with one of the several medical coding protocol or coding schemes. The coding protocol may include procedures for oversight and review of the accuracy of the coding assignments to the health care provider's PMR or EMR. The coding information may be submitted for review, approval and payment by a third party payor. The assignment of codes to the patient's medical record coding is separate from the clinician's creation of the patient's medical health care record. The coding information is frequently not retained as part of the patient's PMR or EHR. Also the assignment of codes to the clinician's notes is performed hours later, sometimes at the end of the clinician's shift or performed separately by nurse or medical coders. Again this timing sequence prevents the assigned coding from being used as an index for a real time EMR or EHR search.

### SUMMARY OF DISCLOSURE

This disclosure includes teaching a system for providing immediate patient medical history and information at the onset of a patient encounter. The immediate access to this information will be invaluable, particularly in an emergency room setting where the health care provide likely has no knowledge of the patient's medical history. It will be appreciated that the health care provider can observe only the patient's symptom. Critical time must be spent on examination and testing or examination procedures (e.g. X-rays or imaging) before a picture of the underlying causes can be assessed.

The teaching of this disclosure are also invaluable in the admitted rare but real exigent circumstance where a scarce life saving medical resource must be allocated among competing patients. For example, information regarding the patient's medical history, e.g., history of respiratory condition and disease, may be relevant in determining whether an individual patient should receive use of a ventilator when another patient, also requiring a ventilator, does not have an adverse medical history. Sadly this is a real occurrence at the time of submission of this disclosure.

The teachings of this disclosure include a diagnostic and treatment software tool that allows electronic recording of initial or subsequent patient encounters wherein the clinician notes may be recorded with appropriate and accepted coding protocols and optionally code descriptors. The software of this disclosure correlates the health care provider's (herein after clinician) text notes with the appropriate alphanumeric codes. The alphanumeric codes (each correlated to a separate and medically recognized topic) are uniquely suited to be used as an index or search tool for an electronic search of the patient's electronically recorded medical history. This electronic search can be initiated and relevant search results returned and displayed to the clinician, e.g., an emergency room doctor, in real time. (By “real time” or “on the fly”, the Applicant intends a time lapse of only seconds or minutes. The Applicant's use of the term “relevant search results” will be appreciated to mean that a search using codes correlated to symptoms of shortness of breath and chest pain will likely not return information regarding the patient's prior ankle surgery.)

The disclosure also teaches adopting these software suggested correlated codes into the clinician's notes of symptoms, diagnosis and treatment. The combined diagnostic and treatment notes, containing such codes, create a more complete patient specific EHR utilizing standardized terms. This more robust EHR containing an alphanumeric or numeric coding index may be used to electronically search the patient's history to corroborate or assist in future diagnoses and treatments. With instant access to the relevant portions of the patient's history, the clinician can treat in real time the cause of the patient's malady in contrast to merely responding to observed symptoms.

The device or tool of this disclosure, by providing instant access to the relevant portions of the patient's history may confirm the clinician's initial diagnosis, thereby expediting the start of treatment, and avoiding delay waiting for lengthy (and costly) test results.

This disclosure teaches integrating established and accepted code protocols used to categorize diagnoses, treatments and tests with the specific medical information or applicable portions of the patient's electronic health record (EHR).

There are 4 primary code sets or protocols. Each is discussed below:
CPT© Standardized reporting of medical, surgical and diagnostic services
HCPCS Level II Used to report procedures, services, supplies, drugs and equipment
ICD-10-PCS Used to report inpatient procedures (hospital)
ICD-10-CM Used to report diagnoses for patients of inpatient or out patient providers.

The Current Procedural Terminology (CPT) code protocol has been developed in the U.S. by the American Medical Association. CPT® is a registered trademark of the American Medical Association. The CPT have been created to standardize reporting of medical, surgical and diagnostic services and procedures in both the inpatient and outpatient setting. The AMA update the CPT code protocol annually. Updates include term nomenclature or medical language to reflect updates in medicine as well as updates in medical terminology and term usage. It invites providers and organizations to participate in the ongoing maintenance of the code set, welcoming those who use it to suggest changes to codes and code descriptors. CPT codes are currently used to report procedures and services to federal and private payers for reimbursement of rendered health care.

CPT codes consist of 5 characters. The majority of the codes are numeric but some codes have a fifth alpha character such as a F, T or U. Examples of CPT codes include:
33275 Transcatheter removal of permanent leadless pacemaker, right ventricular
3006F Chest X-ray results documented and reviewed (CAP)
0510T Removal of sinus tarsi implant
0079U Comparative DNA analysis using multiple selected single-nucleotide polymorphisms (SNPs), urine and buccal DNA, for specimen identity verification.

It will be appreciated that use of standard terminology of diagnoses, testing and treatments utilized by the code protocols will facilitate adoption of standard terms and descriptions being used by clinicians.

Another medical coding protocol widely used in the International Classification of Diseases (ICD) code protocol development by the World Health Organization and adapted for use in the American health system. The ICD codes are used to capture medical diagnosis and procedures information about patients. The current version of the ICD protocol is ICD-10 and contains over 70,000 entries. An updated ICD code (ICD-11) has been promulgated and adopted. Member countries will start reporting using ICD-11 in January 2022.

There are two code sets within ICD-10 (and its predecessor ICD-09). ICD-10 comprises ICD-10-CM and contains diagnosis codes. ICD 10-PCS which contains medical procedure codes. ICD-10 codes are 3 to 7 characters in length. ICD-09 are 3 or 4 digits and appear in the format XX.YZ with an optional fifth digit. The ICD-10 code protocol contains approximately 5 times the number of ICD-9 codes.

The US Medicare system utilizes a variation of the CPT code termed HCPCS coding. There is a HCPCS level I coding protocol which is essentially the CPT codes. The HCPCS level II code that includes orthotic and prosthetic procedures, hearing and vision services, ambulance services, medical and surgical supplies, drugs, nutrition therapy, durable medical equipment, outpatient hospital care, and Medicaid.

This disclosure also teaches components or devices that allow health care providers (elsewhere termed “clinicians”) to electronically enter data of patient encounters, such as identity, symptoms, diagnosis or treatment. In response to the data entry, the device may display software suggested code protocol entries appropriate to the entered data, e.g., symptoms or diagnosis. The suggested codes may be adopted by the health care provider. The codes may then be added to the entry of medical data as part of the EHR. The codes then become an alphanumeric index to facilitate future searching of a patient EMR or EHR.

This disclosure includes expanding patient electronic health records (EHR) stored or recorded by physicians, hospitals, clinics, laboratories, etc., (hereinafter “health care providers” or “clinicians”) to incorporate industry coding protocols, correlating the EHR described diagnosis, treatment or service. It is appreciated that these coding protocols are currently used to facilitate payment to the health care providers from third party payors, i.e., health insurance companies, Medicare and Medicaid.

A further object of the disclosure is the clinician's review and adoption of the correlated suggested codes. This concurrent assignment of codes with the rendering of services facilitates the accuracy of the coding needed for submission of an acceptable invoice to the third party payor. It will be appreciated as discussed further below that current practice is to have separate individuals (medical coders) later interpret the clinician's notes to assign billing codes. The coding protocol can also contain text descriptors that will appear on the invoice and provide useful information to the patient.

This disclosure also expands search access beyond the patient medical records (EMR) retained by an individual physician, clinic or hospital. The disclosure includes mechanisms, e.g., API, allowing real time electronic access to medical records of the patient existing beyond the walls of the individual health care facility, i.e., an EHR. In an embodiment, the disclosure allows communication across different EHR software services or providers, e.g., between Epic and Cerner.

The expanded use of such coding protocols as taught by this disclosure will also facilitate the ability of health care providers to search and access patient histories from patient EHRs. The coding, now incorporated into the patient specific EHR as taught by this disclosure, will provide an index to search the EHR for similar past diagnoses or treatments. A medical health care provider can request patient specific information utilizing the appropriate code or codes to extract the relevant EHR records without having to parse through a voluminous totality of health care records or guess of the previously used key word descriptors. It will be appreciated that much of contents of the health care records may not be relevant to the instant medical event.

Accordingly, in one embodiment of the disclosure, the treating physician, e.g., emergency room doctor, may be able to quickly learn of the patient's relevant medical history by code searching of the patient's EHR. It will be appreciated that in one embodiment, a clinician used key word may be matched to a code descriptor assigned to a specific alphanumeric code.

In another embodiment of the disclosure, Artificial Intelligence (AI) or machine learning may be utilized to correlate notations of symptoms, diagnosis, treatment or tests to the established coding protocols. This may use clinician noted terms and searching for synonyms or related terms utilized or defined in medical literature and dictionaries to correlate to the established code protocols and particularly to the appropriate code descriptors. This knowledge database may be refined over time by continuous review of noted clinician text (vernacular or nomenclature) and selected codes or proscribed treatments and tests.

The disclosure includes comparison of existing EHR records with existing and correlated invoice data (wherein the invoice data includes the assigned code). This step can correlate and assign the codes to clinician's text notations of an EHR with the previously assigned and accepted invoice codes to bolster or expand the database responsive to a clinician's search of EMR or EHR utilizing the codes relevant to clinician text notations. It will be appreciated that some EHR records do not contain an integration of the coding protocols. However the contemporaneous invoicing records very likely contain such coding information. Therefore invoicing resulting from medical services in 2009 will likely contain the codes relevant to the medical services provided. By examining the invoicing records for 2009, the software will correlate the invoicing to the EMR or EHR relevant to the invoiced services. It will be appreciated that the software subject of the disclosure may perform a two step process, i.e., first searching the invoicing records of relevant codes and second, performing a search of the EHR records correlated to invoices. This step will constitute indexing past records within the EHR with the code protocols to facilitate current or future searching.

Also, the software subject of this disclosure may be utilized to retroactively annotate existing health care records with the appropriate codes from the established code protocols. This can be independent of the information or codes that may be contained in the invoice records. It will be appreciated that the software program will have been refined utilizing machine learning from past and current EHRs and accepted coded invoices correlated to the EHRs. Thus existing databases can be enhanced to be utilized in later (current) searches of past medical histories initiated by clinicians. Stated differently, existing EHR records can retroactively be supplemented with the applicable alphanumeric codes. These codes may have been assigned to the past services in the invoicing step. As supplement, the EHR contains the code protocol that can be used for real time electronic searching.

The disclosure also teaches a method of expeditiously and reliably assigning relevant coding protocols at the time a health care provider may be entering patient examination notes, surgery or procedure summaries, etc. It will be appreciated that the number of accepted and maintained code sets may exceed 70,000 codes with individual code descriptors. The quantity of possible codes makes current bill coding practices tedious and time consuming for both the medical coder or the medical health care provider. This also results in mistakes, delays, incorrect payment, etc. This disclosure may reduce these issues.

It will be appreciated that often current practice for medical coding is that medical codes are subsequently and separately added after the patient encounter or treatment. The code entry is often performed by medical billing coders who are not trained health care providers. This sequence and practice results in numerous incorrect code entries, causing numerous billing errors, payment delays and over/under payment.

Similarly, this difficulty may exist when the code protocols are used to index and identify diagnosis, treatment and test results that may be relied upon by other (subsequent) medical health care providers. Accordingly, the disclosure also teaches a method of auto identification and suggestion of applicable/relevant codes at the time the examination notes, etc. are created. The codes provide an alpha numeric sequence (currently up to 7 characters in length) combined with a brief description of the medical diagnosis, procedure or test, i.e., text or code descriptors.

An object of the disclosure may be to supplement the clinician's entry of patient symptoms or draft diagnosis by the suggestion of responsive code entries. Using AI or machine learning, the software can suggest possible treatments or tests to the clinician based on the clinician's entry. This supplemented information may comprise the alphanumeric codes and code descriptors for the suggested treatment or test. These suggested treatment or tests may be displayed to the clinician for evaluation. This additional information displayed to the clinician may alert the clinician that her initial diagnosis entries “were headed down the wrong path” and additional notation may be merited. This supplemental information can serve as a check of the initial text entry. The clinician's rejection of suggested tests or treatments may also be used in machine learning to become smarter and perhaps to not suggest certain test/procedures in response the clinician's notation of symptoms or diagnosis.

Another object of the disclosure is to include the suggested tests/procedures (regardless of perhaps not having been displayed to the clinician) as part of the clinician requested electronic search of the patient's EHR. This inclusion will broaden the search categories. For example, a patient may not have been previously diagnosed in the manner of the current clinician, but has received testing or undergone treatments that would be very useful to the current clinician to know as part of her assessment of the patient. Again, it must be recalled that the subject electronic search is to be performed in real time, thereby the clinician will have the history prior to the return (or ordering) of new and perhaps duplicative tests or procedures.

Stated differently, use of this expanded search criteria may return relevant results from the patient's EHR that may not have been responsive to a search limited to the clinician's notation of symptoms or diagnosis as supplemented with the alphanumeric codes.

It is another object of the invention to allow the software to not only suggest alphanumeric codes (ICD, CPT or HCPCS codes), but to also identify and display the third party reimbursement for each code. This reimbursement information may be calculated utilizing published guidelines, e.g., Medicare reimbursement rates modified by geographic location, or from a database of past reimbursements for the same code. As will be discussed, the identity of the patient's third party payor is typically determined prior to medical services being provided. Third party payors include health insurance companies. Display of this agnostic patient information (reimbursement paid for medical services for other patients wherein the services have the same alphanumeric code but without identification of patient identities) may serve as further check for the clinician that her symptom or diagnostic notes are being accurately correlated by the software to the correct code(s).

The software therefore can provide extensive information to the clinician based upon the notations of symptoms and initial diagnosis. This information includes the correlated medical coding protocol with text descriptors, related suggested possible treatment and tests (again correlated by the medical coding protocol) and the historically determined reimbursement rates associated with the menu of likely services.

In addition, the software provides a search tool utilizing the appropriate codes to identify relevant patient information from the EHR. The search is conducted electronically and in real time. The search results, relevant past patient medical encounters, test results and treatment, can be displayed to the current clinician. It will be appreciated that in some situations, the clinician may have no patient history and is able only to treat the observed symptoms with no clue as to the underlying cause.

The disclosure also teaches devices such as electronic tablets, e.g., iPad, or smart phones containing software subject of this disclosure and programed to accept entry of medical data from a clinician and responsively suggest and display one or more code protocol entries. These entries are available for possible adoption by the health care provider into the EHR as well as for medical expense invoicing. Also the device, having received medical code entries from the clinician, may be used as a platform to initiate a search of the applicable patient's EHR for similar past medical events, thereby allowing the clinician to receive the patient's medical history in real time. The disclosure includes utilization of the device, e.g., display screen of the tablet, smart phone, etc., to convey the patient's medical history as search results.

For example, if the patient exhibits symptoms of a knee injury, the current ICD-10 codes S83.4 and S83.5 may be displayed along with the text descriptor to the clinician. If deemed appropriate, the codes may be adopted into the patient's EHR. Also an electronic search may be initiated via the device. Entry of these codes would return any past patient records of sprains of the cruciate ligament of a knee and sprains of collateral ligament of a knee. Entry of code S83.20 will provide information of past diagnosis of tears of a meniscus of the patient's knee. Note that specifying the 4 character codes will return entries of all underlying codes, i.e., entry of code S83.4 will also return an entry of S83.41. The software may also suggest other possible causes pertinent to the clinician's entered description of symptom. This suggestion may assist the clinician.

The disclosure teaches a system for improving the efficiency and reliability of entering correct codes for each diagnosis, treatment or test by a coding protocol or program that auto suggests coding entries in response to the text added by the health care provider creating the EMR. Alternatively, the program may suggest one or more codes based upon the totality of the health care provider's entry. The coding entries can be alpha-numeric, alpha or numeric codes contained in a medical provider's service code protocol.

The operator (medical coder or medical health care provider) may enter a description of diagnosis, treatment or testing into a particular data area utilizing a key board and entered data may be displayed on the portable or fixed device screen. A dynamic list of possible codes is generated and displayed. The listing is based on entered text for the diagnosis, treatment or testing, etc. In one embodiment, the suggested (or selected) codes may appear within the entered text or in a separate designated area. It will be appreciated that the data entry for coding and recording will be performed with a key board and display screen. For example, the data entry field may be accompanied by a column, row or machine fillable block (“code space”) located to the left or right of the data entry field. The data entry field and code space may be expandable to allow unlimited text content. The code space may be designated as “code”, or “diagnosis, treatment, test code” or similar. As the clinician enters text into the data field, possible codes may be simultaneously displayed in the designated code space. As the diagnosis, etc. is entered, suggested codes may appear in the code space. The clinician may select one or several alternate codes for recording with the EHR.

The suggested code selection may be based upon text entered by the clinician. As the clinician enters characters of text data item, the list of corresponding diagnosis, treatment or test codes are searched for an entry that is appropriate for or matches the entry typed in the data field. If a code match is found, then the matching code (or codes) is displayed in the code space to the clinician as a suggested completion. The clinician can then elect to accept the suggested completion or to continue entering the data item. The coding may be separately inserted. However that coding input will be recorded into the EHR and may be utilized as an indexing tool for later searches.

The disclosure may also include incorporating an auto correct function. As text is typed or otherwise entered into the data entry field, the program may automatically suggest a completed word or entry. The auto correct function may correct spelling or grammatical errors. The suggested entry may match a text description that accompanies an alpha-numeric code. A code protocol can comprise the alpha-numeric code plus a text descriptor. This feature can save clinician time.

The ICD, CPT or other protocol utilized by third party payor coding scheme can be used to provide clinicians with an index that facilitate the clinician obtaining timely access into a patient's voluminous EHR.

This disclosure teaches a system algorithm for classification and indexing of patient electronic health records (EHR). The algorithm utilizes continuously updated code descriptors or code classifiers wherein the applicable alphanumeric codes, described in the code protocol, are assigned to a patient's electronic health record (EHR) entries. The EHR entries are created by clinicians in describing or listing diagnoses, treatment procedures and testing information. The indexing can facilitate identifying prior health diagnoses, treatments and tests for the patient that may be related or relevant to the patient's current medical event. The index identifies the parts of the body that have been affected, the nature of the health issue, any treatment performed or tests (and test results) that have been conducted.

One of the goals of this disclosure is to utilize existing medical billing code protocols to provide a ready and consistent index to patient electronic health records (EHR). One manner of accomplishing this goal is to incorporate the assigned billing codes into the EHR. The codes can be embedded into the transcript of the patient's prior diagnosis or used as part of an EHR section header. The incorporated code may include the code descriptor. The code descriptor may, in one embodiment, contained the amendments or annotations created by the prior clinician. The alphanumeric code is intended to be closely associated with the relevant and corresponding portions of the EHR entry created by the clinician. Searching the patient's EHR by code number will lead to the notes, treatment and test results that pertain to the medical conditions assigned to the specific alphanumeric code or related alternate codes.

The disclosure also includes an EHR system wherein the records are annotated with the applicable codes from the protocol. It will be appreciated that such an EHR system will be readily searchable. The EHR system and method of creating such system is disclosed and may be accomplished by the use of the tool and method of use.

In one embodiment, it is the goal that the current clinician can receive the information of past (and alphanumeric code indexed) medical examination, etc., in real time during, for instance, the current initial examination of the patient. The electronic records can be electronically recognized, parsed and the responsive portions be communicated to the requesting clinician without human intervention. In an embodiment, the information can be displayed on a clinician's device, e.g., iPad. Again cryptography such as public and private keys may be utilized to ensure the authenticity of the received records. Similarly, cryptography can be used to ensure the requesting clinician is authorized to receive the information extracted from the EHR. The validation measures may include block chain technology to ensure the validity and authenticity of the extract portion of the patient's EHR.

It is one goal of the disclosure that the current clinician's electronically recorded observations of the patient's condition and resulting diagnosis become an entry into the patient's EHR. The electronic interface device may allow access to a network or system containing the patient's EHR. The entry may be indexed by the applicable alphanumeric code as well as by date. The alphanumeric codes are assigned to separately identified medical conditions utilizing recognized industry coding protocols. For example, it will be possible to learn of treatment for knee injury by entering search terms consisting of one or both text entries, e.g., knee pain, knee swelling, etc. or by code entries, e.g., ICD-10 codes S83.4, S83.5, etc. The EHR can also be searched by date, e.g., date of treatment, test or diagnosis, etc.

It is a goal that the clinician's code indexed entry into the EHR be contemporaneous with the clinician's examination of the patient. The clinician's entry of notes or diagnosis may allow an algorithm of the disclosed software to assign one or more codes from an industry accepted code protocol at the time the entry is created. The disclosure includes the algorithm of the system suggesting applicable codes to the clinician based upon the text of the clinician's notes, etc. The suggestions are to be enhanced by the system algorithm having learned the correlation of terms used by the clinician with the code protocol.

Therefore the disclosure includes machine learning capabilities wherein the algorithm associates and integrates past data entries with adopted code selections. This will allow subsequent data entries to prompt a revised (and more responsive or applicable) listing of suggested codes. This learning may be device specific (reflecting data entries and code selections of an individual clinician) or system wide.

It is yet another goal for the clinician to be able to promptly request a search of the patient's EHR for similar prior events or occurrences. For example, it will be helpful for a clinician examining and treating a patient complaining of knee pain to know that the patient has previously had a surgical procedure to repair a torn meniscus of his/her knee. For example, the current clinician may quickly learn whether the complained event is a new injury or an aggravation of an old injury. Further the clinician may learn of past unsuccessful treatment. This may allow the current diagnosis to be more accurate and to allow more effective treatment.

Therefore the disclosure teaches an embodiment wherein the clinician may initiate a search request, via his/her electronic interface device, of the patient's EHR based upon the codes assigned to the current patient observations or diagnosis. The search request may be initiated upon the clinician authenticating his/her authority for access to the patient's EHR. The search request may include procedures to ensure that any received information is valid and authenticate. In an embodiment, the clinician may be able to initiate the search request without having to leave the device electronic screen displaying the clinician's current diagnosis. Stated differently, the clinician's draft notes and diagnosis, correlated with a suggested or accepted alphanumeric code(s), may serve as the search request.

Another goal is that the suggested codes include the generic code as well as the species that may be selected by the clinician (or modified by amendment of the code descriptor). For example, a code indexed diagnosis for pain in knee may include broad generic codes for knee as well as one or more species codes for specific to the clinician's diagnosis. The device subject of this disclosure may allow the clinician to tailor a search broadly or narrowly.

It is another goal to provide a system in which the clinician can only enter his/her notes of observation or diagnosis after the clinician is identified and authenticated as having access to the patient's EHR. This may be performed by several different steps, including a two factor formulation or authentication procedure. The advantage of this embodiment include that user login, i.e., the clinician, utilizes two stages: a first stage, including anonymous authentication and a second stage, including identifying information authentication, in which the user needs to provide a user name and password for authentication.

This application pertains to a tool and method for accessing a repository or database of electronic health records, i.e., EHR records. This application also pertains to a repository of electronic healthcare records that may be accessed electronically and electronically stored health care records (EHR) of individually identified patients searched for specific terms or medical conditions, diagnoses, medical procedures or treatment, or testing such as laboratory tests, imaging, etc.

The EHR records of multiple patients may be collectively stored in an EHR repository. The records may be searched by individual patient name or other identifiers such as but not limited to patient name, date of birth (DOB) or social security number (SSN). The EHR records may alternatively access through a health information exchange (HIE). The EHR records may be accessed directly from separate offices, clinics or hospital utilizing differing EHR services, e.g., Epic, Cerner, etc., utilizing application program interfaces or similar technology.

Multiple EHR or EMR records for an individual patient may be stored in chronological order.

The EHR records may contain information regarding health events and medical encounters with clinicians. The EHR records may contain diagnoses, medical procedures or treatments or laboratory test results or imaging, e.g., MRI or X-ray. Each entry of diagnosis, procedure or testing may be annotated or accompanied by an alphanumeric code. The code may be correlated to and identify a specific diagnosis, treatment or test procedure.

The application includes a system of components that allows a clinician to (i) identify the patient, (ii) record a draft for final diagnosis, (iii) enter procedures or test performed, etc. The recording components (elsewhere termed “electronic device” or “user device”) can electronically transmit the recorded patient information to an EHR repository. The transmitted information may be added to the chronological listing of EHR events. The device can also be used to transmit the information to cause a search to be conducted of earlier recorded EHR events for possibly related medical treatment or tests.

The EHR records, created by the device for (i) memorializing the diagnosis and treatment of a medical event or (ii) searching past EHR records within the repository for related or similar diagnoses, treatments or tests, may include alphanumeric codes correlated to or representing/identifying specific medical diagnosis, treatment or testing. The prior EHR records may incorporate similar alphanumeric codes. The function of searching the repository for related or similar diagnoses, treatments or tests may utilize the coding protocol for identifying the diagnoses, treatments or tests.

The search results, incorporating the same or similar alphanumeric codes, may be transmitted to the requesting party having initiated the search, thereby allowing a clinician to learn the patient's history of medical conditions or treatments. This will also avoid duplicative tests, lost time and waste

The user device subject of this disclosure may be the user device described in the related applications identified above.

The system also includes the transmitting components such as servers, the Internet, an intranet or internal network, and an EHR repository accessible to the device utilizing communication components. The system may also comprise public key infrastructure. The disclosure also includes a method for promptly searching and retrieving medical histories for real time use by a health care provider.

### BRIEF SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1 illustrates one embodiment of the patient examination, diagnosis and treatment cycle with designation of applicable ICD codes from the coding protocol. Included are steps of initiating a search of the patient's EHR using primary & secondary ICD codes and initiating an invoice for services utilizing the primary ICD codes.

FIG. 2 illustrates an embodiment wherein the patient is examined and a diagnosis assigned utilizing the ICD coding protocol, including optional assignment of additional secondary codes that may be relevant to the diagnosis or follow-on treatment. Treatment can be initiated as identified by and incorporating ICD or CPT codes with initiation of search and invoicing using ICD or CPT codes.

FIG. 3 illustrates an embodiment of the health care provider entering text (patient observations and diagnosis information) to an EHR with the device suggesting applicable codes (with code descriptors) and device learning modified code descriptors (code identifiers) from accepted text and codes.

FIG. 4 illustrates an embodiment of the method subject of the disclosure including a search of an EHR repository utilizing patient ID and selected codes.

FIG. 5 illustrate an example of a logic flow step wherein the clinician's entry is parsed by the system of the disclosure to suggest relevant diagnostic codes, selection of codes, and optional system selection of relevant treatment and test codes for EHR search.

FIG. 6 illustrating a further embodiment wherein the system simultaneously parses the clinician's entry for context and for matching keywords contained in the codes wherein the dual activities are combined at the initiation of the search.

FIG. 7 illustrating the system utilizing machine learning to correlate selected clinician diagnosis codes with relevant test or treatment codes and the aggregate of such codes are subject of the search. In an option, the system suggested test/procedure codes are displayed to the clinician prior to initiating the search.

FIG. 8 illustrating the steps of displaying past reimbursement based upon EHR entity database.

FIG. 9 illustrates clinician's entry of text, etc., based upon diagnosis, procedures or tests, optional search request to network, etc., review of draft EHR entries and optional search results. Also illustrated is the coding decision transmitted to a document manager and submission of final EHR entry to a file storage data base. Note that the file storage may be a distributed network wherein each node of the network possesses an identical copy of the patient EHR.

FIGS. 10a-10f illustrate one embodiment of a screen display of the device subject of this disclosure wherein the provider's (clinician's) entries are displayed (with auto correct and auto completion features) and auto or manual filling of codes applicable to the provider's text entries. In the illustrated example, the clinician's notes are contained on the right hand side of the screen display and the suggested codes are displayed in a left hand panel of the screen display. The Figures illustrate the system use of machine learning to select/suggest diagnostic as well as treatment and test codes.

FIG. 11 illustrates an embodiment of the clinician interfacing with a document manager, including the clinician inputting text information (right hand side of screen) and presentation of possible/suggested coding decisions on the left hand side of the screen. Also shown is the data entry information for utilizing the information presented on the display screen to initiate an optional search function. Information includes patient identifying information as well as identity of clinician requesting search. The display screen features scroll bars to allow greater input and display of data. Also featured is a single click search button.

FIG. 12 illustrates an embodiment of the clinician's system interface wherein a clinician initiated search utilizing machine learning suggests a possible diagnosis and related relevant code entries.

FIG. 13 illustrates an alternate embodiment of a display screen for review of data returned from a search. The clinician may scroll through the documents. Also shown are components to accept or reject the suggested code, submission of the accepted code and selected document notations for entry into the patient EHR.

FIG. 14a illustrates an embodiment wherein the contents of a patient's Electronic Health Records (EHR) can be classified into class and multiple subclasses, etc., by entry of subject matter utilizing a coding protocol such as the Classification of Procedural Terminology (CPT). “EHR File Storage/Document Collection” may include a patient EHR or draft EHR.

FIG. 14b illustrates an embodiment of a classification system wherein an EHR document (presumably uncoded) is created or retrieved. The document may be presented to the user and a user coding decision made. Documents or text may also be machine reviewed for suggested coding into a class or subclass.

FIG. 15 illustrates an embodiment showing a relationship between multiple health care providers (clinicians or users), the user device (including display, processor, storage device and input or interface device) and a network of possible health care entities possessing patient EHR documents. The network may constitute a distribution network wherein each entity contains identical copies of a patient EHR. This can include transmission and storage of new entries to the EHR. Thus the system may function as a distributed network.

FIG. 16 illustrates a further relationship between the processor of the health care entity possessing EHR documents and the components of the entity storage and user device.

FIG. 17a illustrates an embodiment of the steps wherein the health care provider requests that past EHR entries be searched utilizing the accepted codes as indexing components with the accepted diagnosis, text, etc. Illustrated is an embodiment wherein the health care provider selects the entities from whom a search is requested. Also illustrated is an embodiment of the steps for identifying and authenticating the requesting health care provider prior to receiving EHR documents. Authentication information and privacy protection components are also illustrated.

FIG. 17b illustrates a more detailed description of authentication and privacy steps required in order to receive EHR extracts matching or relevant to the code indexed request.

FIG. 18 illustrates one embodiment showing the relationship between the clinician's device display, input or interface component and the information classification system.

FIG. 19 illustrates an embodiment of the steps to request a medical record from an EHR repository. The method includes optional steps for using a user name and password of a clinician as well as authenticating a user device such as a tablet computer, CPU, smart phone, etc. Note this embodiment also includes the clinician supplying identifying information at the initiation of the process. This is illustrates another embodiment for clinician/requestor identification and EHR privacy.

FIG. 20 illustrates an embodiment utilizing a PKI infrastructure, including the clinician transmitting an authorization certificate along with a public key at the start of the request process to the EHR repository.

FIG. 21 illustrates an embodiment of a PKI infrastructure.

FIG. 22 illustrates another embodiment of a PKI infrastructure. Included is the optional Online Certificate Status Protocol, Certificate Transparency Log and Root CA.

FIG. 23 illustrates an embodiment of the components of the system utilizing optional encryption and steps of EHR data source processing the request and returning requested search information.

FIG. 24a illustrates utilization of the QR code.

FIG. 24b illustrates use of the signature encryption and cyphertext authentication module.

FIGS. 24c & 24d illustrate extraction steps of the cyphertext encryption module.

FIG. 24e illustrates an embodiment wherein a clinician device transmits a login request to a repository system.

FIG. 25a illustrates a flow path utilizing the QR cyphertext methodology.

FIG. 25b illustrates a two step clinician authentication protocol.

FIG. 25c illustrates the first step of the two factor authentication protocol.

FIG. 25d illustrates detail of the second step of the two factor authentication protocol.

FIG. 25e illustrates the relationship of the Verification Code Authentication Module to the Username and Password Authentication Module.

FIGS. 25f & 25g illustrate a further embodiments of the two factor authentication protocol.

FIGS. 26a, 26b & 26c illustrate a computer and computer environment suitable for supporting the operation of a device embodiment subject of the disclosure. FIG. 26c illustrates a flow chart for a autocomplete function.

FIG. 27 illustrates the operations of an autocomplete function that may be used by the disclosure and which is incorporated into the display of one embodiment of the device subject of the disclosure.

FIG. 28 is a further illustration of an embodiment of the operation of the autocomplete command.

FIGS. 29a & 29b are further illustrations of an embodiment of the operation of the autocomplete command. FIG. 29a expands the description of a step illustrated in FIGS. 26c and 27. FIG. 29b is an expansion of a step illustrated in FIG. 29a.

FIG. 30 illustrates an autocomplete algorithm using input parameters. It is an expansion of a step illustrated in FIG. 4c.

FIG. 31 illustrates a flow chart of one embodiment of the disclosure. The relationship to current coding/invoicing practice is shown.

FIG. 32 illustrates a flow chart for identifying a medical term within the clinician notes utilizing keyword searching and code protocols.

FIG. 33 illustrates a flow chart wherein keywords are extracted from patient encounter notes and sequential searching of medical term fields utilizing an algorithm with entry into a code protocol.

### DETAILED DESCRIPTION OF DISCLOSURE

Millions of individuals are examined, diagnosed and treated for medical issues each year in the United States. Each physician, hospital, clinic, laboratory or medical testing facility (hereinafter “health care provider”) records the examination, diagnosis and treatment pertaining to an individual patient in either paper or electronic format. Further, most individuals are covered under some form of third party medical service payment system, i.e., health insurance, Medicare, Medicaid, etc. In the past, each health care provider submitted invoices for reimbursement to one or more third party medical payment systems. Each clinician utilized its own system for describing common diagnoses and services.

To simplify the reimbursement process, standardized coding schemes or protocol have been developed. One principal code, Current Procedural Terminology (CPT) has been developed in the U.S. by the American Medical Association. This code has developed succinct but detailed classifications to accurately distinguish among different activities (including differing activities pertaining to the same body part, e.g., knee or kidney). This medical coding scheme is used in invoicing and payment for medical and health care services. Identical treatments, although described in different terms, are intended to be assigned the same billing code. This has numerous benefits, including expediting payments to clinicians. For instance, third party payors are able to assess variations in invoices for identical medical treatment service.

Another medical coding protocol widely used in the International Classification of Diseases (ICD) code protocol development by the World Intellectual Property Organization (WIPO). The current version of the ICD protocol is ICD-10 and contains over 70,000 entries.

Often, the assignment of codes is performed separately from the documentation of a patient diagnosis, treatment or test. The code is used to as billing codes. The codes are not routinely part of the patient's medical record (PMR) or electronic health records (EHR). The codes are assigned by trained individuals frequently termed as medical billing coders. However, medical billing coders are not trained medical professionals. Coders seek to interpret the notation of diagnosis, treatment and testing created by clinicians and correlate the diagnosis, etc. to the code protocol. A numeric or alpha numeric coding scheme such as the CPT or ICD is used by the medical billing coder. Frequent mistakes or mis-interpretations occur resulting in delayed payment etc.

The coders endeavor to assign an appropriate code to the diagnosis and treatment services provided to the patient. Services pertaining to a patient's knee receive different code(s) from the codes assigned for treatment of an intestinal disorder. Surgical services are coded differently that drug therapy. Services such X-ray and laboratory blood work are also coded differently. This pre-established coding scheme is intended to be applied uniformly for the same treatment or diagnosis for multiple patients. One or more codes are assigned to each diagnosis or treatment service provided to the patient. The coding schemes or protocols provide a uniform basis for time and cost efficient review, approval and payment of medical invoices by third party insurance providers. The patient is typically the beneficiary of a health insurance contract with the third party payor.

Increasingly, patient medical records (PMR) are retained electronically by a health care entity and the records are frequently termed electronic medical records (EMR).

One aspect of this disclosure is to provide a diagnostic tool to expand the EMR created by one or several health care providers into a more comprehensive electronic health record (EHR) by encouraging the common use of medical coding schemes or protocol such as CPT that is applied to all patient records created at all (or nearly all) heath care providers.

This disclosure also pertains to making a patient EMR or a physician, clinic or hospital patient records accessible to other clinicians. In this manner, the disclosure may serve as a Fast Healthcare Interoperability Resource (Fhir) and may be consistent with the HL7 standards. The disclosure may work in conjunction with Health Information Exchanges (HIEs) or directly with the health care provider's EMR utilizing application program interfaces (API).

This disclosure also pertains to providing a tool facilitating expansion of the electronic health records (EHR) to correlate the EHR described diagnosis, treatment or service to a third-party medical service providers payment coding scheme. Annotating or recording each clinician's record of diagnosis and treatment with the CPT or ICD, now principally utilized solely as a billing coding protocol, allows other medical service providers with expedited and efficient access to past patient medical records. It will be appreciated that coding typically occurs after the fact (hours or days after the patient encounter and is performed by medical coders, in contrast to a clinician interacting with a patient). Further, the coding assigned for invoicing purposes may ignore specific diagnosis and treatment steps but rather be edited by an entity's, e.g., hospital, coding assignment and review procedures to reflect a general “theme of the patient treatment.” A clinician's diagnostic notes of codes may be omitted from the EMR. The third party payor coding protocol utilized by the clinician as assisted by the software and display devices subject of this disclosure, in contrast to the medical coder's interpretation, can be used to provide medical providers with access to the patient's EHR. The coding protocol can be used as an index to search a patient's EHR.

The patient diagnosis and treatments records contained within the individualized EHR can be indexed by patient identity, e.g., name, SSN, service provider assigned patient number, insurance provider and insurance policy number, etc.

Indexing Clinician Notes into EHR, Adoption of Codes and Focused Searching

This disclosure pertains to a tool that electronic records of a clinician's diagnostic or treatment notes that can be incorporated in the relevant patient's EHR. The tool also displays the accepted diagnostic and treatment coding protocols correlated to the clinician's notes. The codes may be adapted by the clinician. This display and adoption of codes into the patient's EHR occurs at the time of the patient encounter. These incorporated codes create an index of the patient's medical records by the billing coding numbers. This indexing, created by the use of the tool, will allow a medical service provider to search and receive past patient's treatment records by the service (diagnosis, treatment, laboratory test results, etc.). The relevant past medical service records are accessed by utilization of the uniform service coding protocol. The relevant past medical records can be accessed via this diagnostic and treatment tool. The coding protocol, utilized to facilitate medical payment, can be expanded to be used to search individual EHR records. Search by medical treatment code numbers would substantially narrow the scope of review required of health care providers. It would also save time and effort of the past health care providers in searching and copying records. The instant health care provider would have greater confidence in the accuracy of the records received from individual past providers, in contrast to receiving redacted records from one or more central clearing house or third party service.

Requesting medical records by patient name or SSN will result in a totality of all medical records i.e., EHR, pertaining to that patient being provided. However, the party requesting the records (the requestor being another medical clinician providing current medical services related to a past medical treatment) may be interested only in medical records pertaining to a specific medical condition, e.g., the patient's knee or knee injury.

For example, an orthopedic surgeon evaluating treating a patient for complaints of left knee pain may be interested in records pertaining to past left knee orthoscopic surgery. The orthopedic surgeon may not be interested in medical records pertaining to the patient's past hospitalization for pneumonia. In this situation, it would be productive and time and cost efficient to request medical records for patient that pertain past diagnosis, treatment or testing (e.g., X-rays) to patient's left knee. This disclosure teaches a method for prompt receipt of relevant EHR pertaining to the left knee by requesting the EHR by the patient's identification and the relevant diagnosis and treatment codes. The codes utilized for billing purposes also have an expanded utility of prompt and efficient receipt by a clinician of past diagnoses and treatment.

The search request could specify records associated with particular billing codes for knee surgery, X-ray or other imaging of the left knee, pharmacologic treatment for inflammation or treatment of arthritis. In one embodiment, the disclosure contemplates treatment and test codes relevant to the clinician's diagnosis will form part of the search. This enhanced searching utilizes artificial intelligence and machine learning to select relevant treatment and test codes. It will be appreciated that this embodiment will cast a broader net for searching of relevant records. The search will not be dependent upon the clinician's adopted diagnosis codes.

In one embodiment, a search request may comprise the patient's name, date of birth and possibly other identifying information such as social security number, address, date of past services (if known) and a consent for release of information signed by the patient. (This consent can be obtained as part of the patient's initial information disclosure to the health care entity, e.g., clinic or hospital.) The search request would also describe the instant treatment issues and possibly the code or codes associated with that medical issue. The recipient of the search request may only be required to locate the records, and submit records responsive to the listed coding information. It of course will appreciated that the full EHR may be later submitted if requested.

It will be appreciated that over time, an individual patient's EHR will increase in size as the patient receives additional medical examinations and treatments for multiple medical events or conditions. It will further be appreciated that the subject matter of the treatments will vary. It is not time or cost efficient for the voluminous totality of past medical treatment records (PHR) be solicited from multiple service providers and then parsed for information pertaining to the instant medical event. Even if the patient's records are at one location, it is not feasible to quickly parse through voluminous records that pertain to multiple diagnoses and treatment to find information specific to the instant illness or trauma.

Under current practice, the providing of medical services includes the steps of (i) patient identification, (ii) identification of patient payment mechanism for medical services, (iii) examination, (iv) diagnosis (v) treatment (vi) submission of records for coding, (v) submission invoice to third party payor for payment, and (vii) record retention. There may also be the step of adding the records to the totality of the EHR. The retained records are not under current practice, however, correlated to the assigned payment coding. However, the payment coding scheme or protocol is intended to have a comprehensive number set for all diagnoses, treatment, surgery, drug therapy and ancillary services such as X-ray, test scans (e.g., MRI) and laboratory work and testing. This disclosure teaches an expanded use of the ICD or CPT codes from merely a tool for classifying treatment and diagnosis for billing but to provide a comprehensive index of medical treatment.

### EHR Coding Protocols

The current practice utilizes one of several existing coding schemes. One coding protocol is the International Classification of Diseases (ICD). The ICD has been created by the World Health Organization. This classification or coding scheme is, like other classification or coding protocols, continually updated as treatment procedures expand and are supplemented. Currently utilized ICD-10 contains over 70,000 separate available code entries. ICD-11 has already been released and will begin implementation in 2022.

The ICD coding protocol (currently identified as ICD-10 for tenth updated revision and implemented beginning in 2015) is broken down into categories and subcategories and may contain as many as 7 digits. The detail of the ICD-10 coding is sufficient to distinguish between the right and left side of a patient, e.g., the coding distinguishes between the right and left arm.

Published examples of the ICD code include:

B01.2 Varicella pneumonia
K21.0 Gastro-esophageal reflux disease with esophagitis

030.003 Twin pregnancy, unspecified, third trimester

The ICD code structure is
a. Characters 1-3—Category
b. Characters 4-6—Etiology, anatomic site, severity, or other clinical detail
c. Characters 7—Extension

Note that for the first entry above, B01.2 is the applicable code. “Varicella pneumonia” is the code descriptor.

The following example shows the more detailed information gained through the added characters.
S52 Fracture of forearm
S52.5 Fracture of lower end of radius
S52.52 Torus fracture of lower end of radius
S52.521 Torus fracture of lower end of right radius
S52.521A Torus fracture of lower end of right radius, initial encounter for closed fracture

In the above example, S52 is the category. The fourth and fifth characters of “5” and “2” provide additional clinical detail and anatomic site. The sixth character in this example indicates laterality, i.e., right radius. The seventh character, “A”, is an extension that provides additional information, which means “initial encounter” in this example. See AMA publication entitled “Preparing for the ICD-10 Code Set: Oct. 1, 2015 Compliance Date. See AMA publication entitled “Preparing for the ICD-10 Code Set: Oct. 1, 2015 Compliance Date”, 2014. These publications are incorporated by reference into this disclosure herein in their entirety.

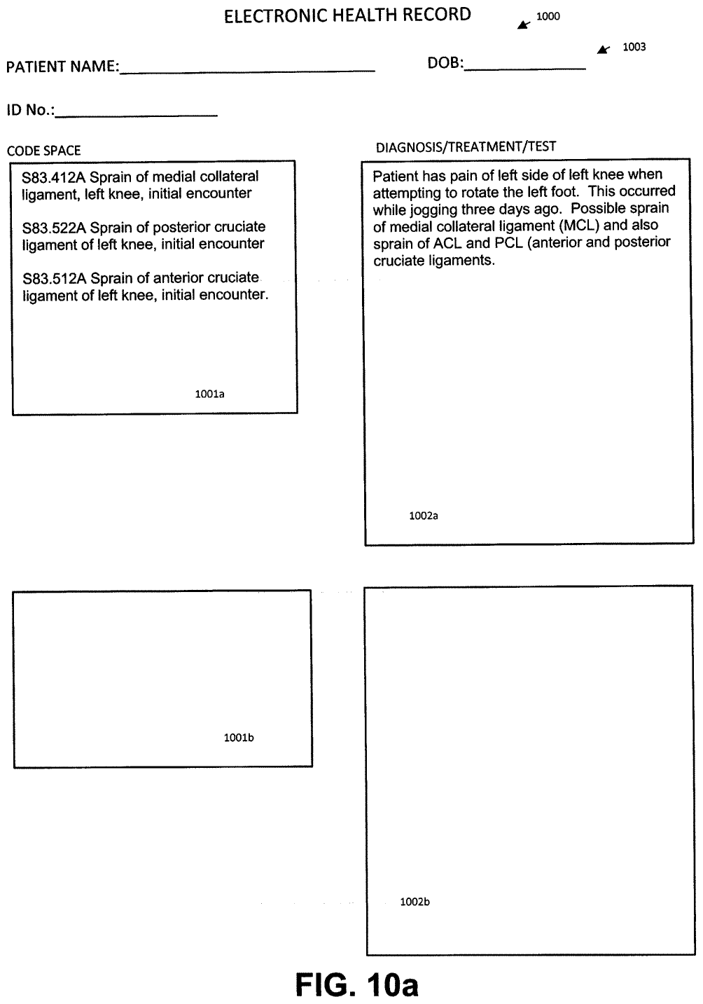

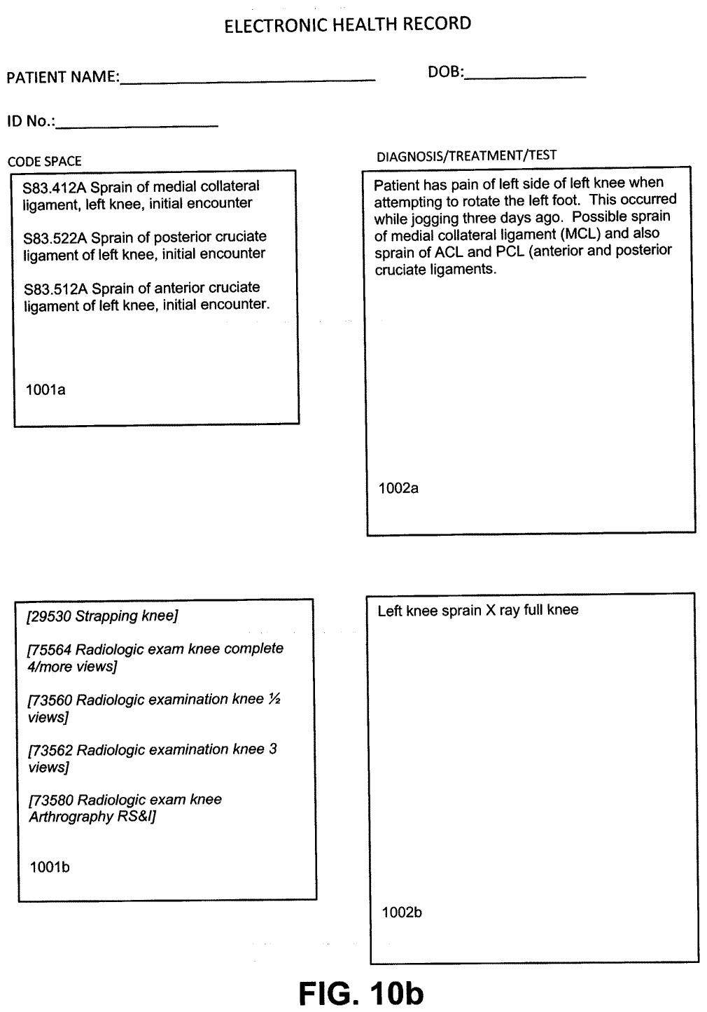

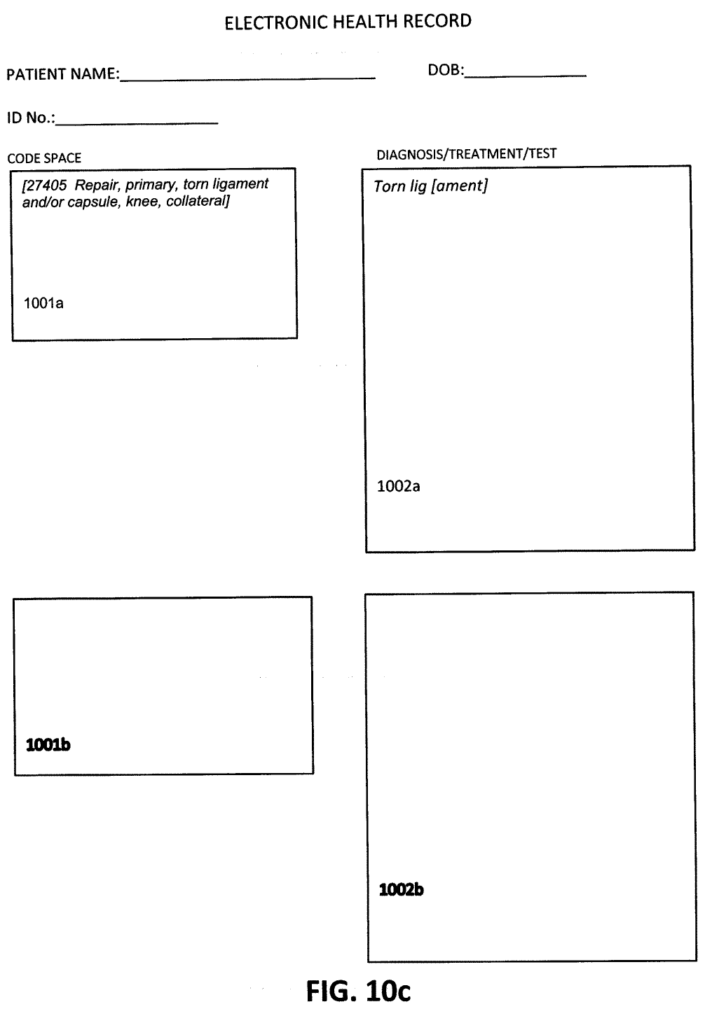

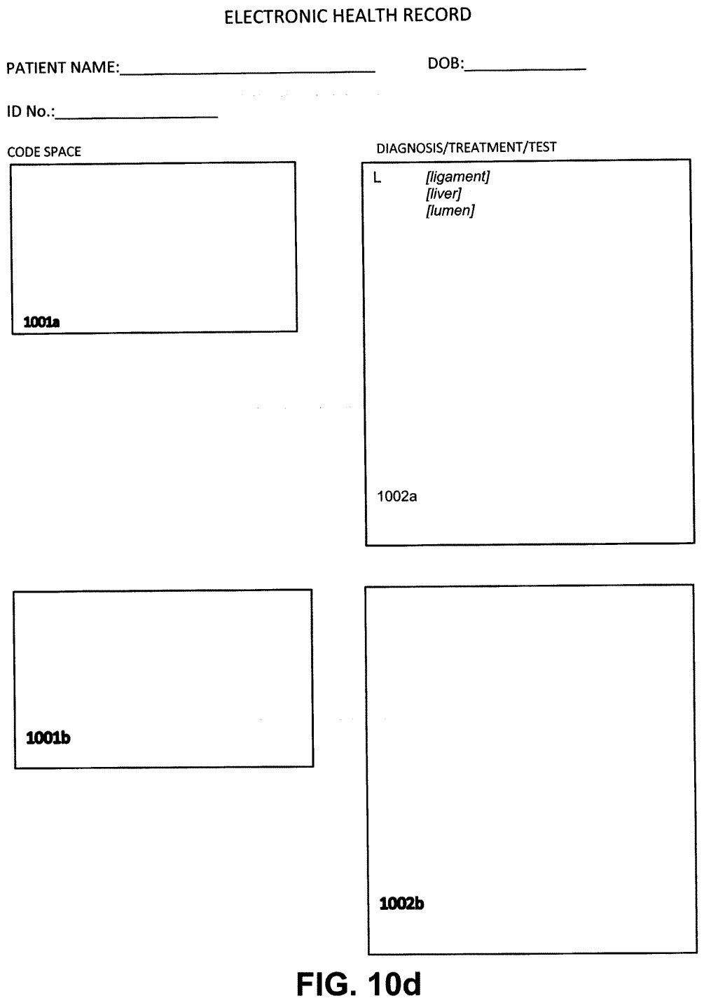

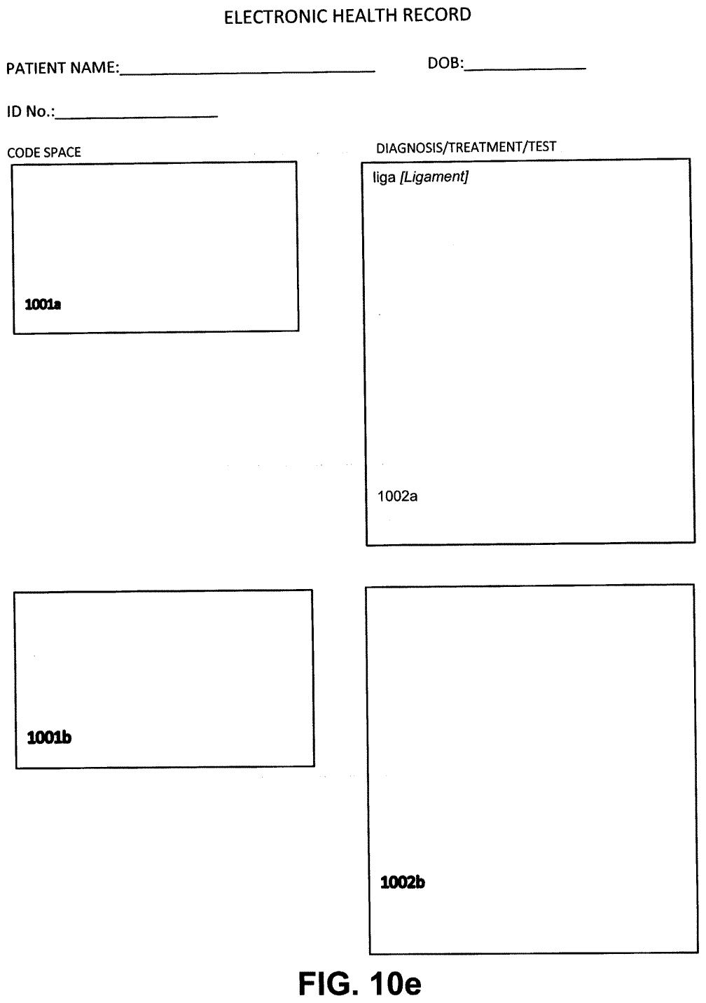

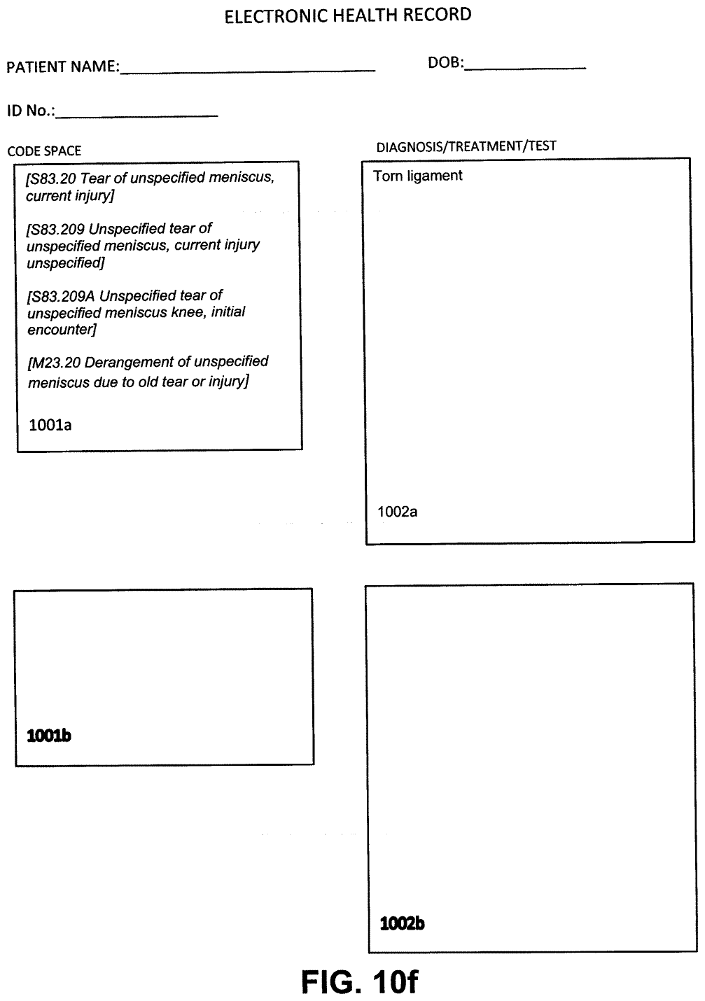

Search of the ICD-10 CM for “knee ligament” returns multiple entries. See FIG. 10. Entries include, but are not limited to, the following examples:

| | | |
|---|---|---|
| a. | S83.4 | Sprain of collateral ligament of knee |
| b. | S83.5 | Sprain of cruciate ligament of knee |
| c. | S83.401A | Sprain of unspecified collateral ligament of right knee |
| d. | S83.4015 | Sprain of unspecified collateral ligament of right knee, |
| | | sequela |
| e. | S83.4015 | Sprain of unspecified collateral ligament of left knee, |
| | | sequela |
| f. | S83.501A | Sprain of unspecified cruciate ligament of right knee, |
| | | initial encounter |
| g. | S83.512A | Sprain of anterior cruciate ligament of left knee, initial |
| | | encounter |
| h. | S83.512D | Sprain of anterior cruciate ligament of left knee, |
| | | encounter subsequent |
| i. | S83.512S | Sprain of anterior cruciate ligament of left knee, |
| | | sequela |
| j. | S83.521A | Sprain of posterior cruciate ligament of right knee, |
| | | initial encounter |
| k. | S83.512D | Sprain of posterior cruciate ligament of right knee, |
| | | subsequent encounter |
| l. | 583.512S | Sprain of posterior cruciate ligament of right knee, |
| | | sequela |

### Primary Difference Between ICD-10-CM and ICD-10-PCS

When most people talk about ICD-10, they are referring to ICD-10CM. This is the code set for diagnosis coding and is used for all healthcare settings in the United States. ICD-10PCS, on the other hand, is used in hospital inpatient settings for inpatient procedure coding.

ICD-10-CM breakdown

Will replace ICD-9-CM
Approximately 68,000 codes
3-7 alphanumeric characters
Facilitates timely processing of claims
ICD-10-PCS breakdown will replace ICD-9-CM for hospital inpatient use only. ICD-10-PCS will not replace CPT codes used by physicians. According to HealthCare Information Management, Inc. (HCIM), “Its only intention is to identify inpatient facility services in a way not directly related to physician work, but directed towards allocation of hospital services.”
ICD-10-PCS also comprises 7 alphanumeric characters
ICD-10-PCS is very different from ICD-9-CM procedure coding due to its ability to be more specific and accurate. “This becomes increasingly important when assessing and tracking the quality of medical processes and outcomes, and compiling statistics that are valuable tools for research,” according to HCIM.

### ICD and CPT Code Protocols

Other coding protocols may be used for classifying treatment, e.g., Current Procedural Terminology (CPT) created and maintained by the American Medical Association. Note that the ICD-10-PCS protocol does not replace CPT codes used by physicians. (The CPT© is a registered trademark of the American Medical Association (AMA). The CPT is also copyrighted and maintained by the AMA.)

It will be appreciated that the existing code protocols have tens of thousands of individual codes. Each code has a code descriptor. Both the ICD and CPT code protocols include multiple categories and levels of classifications, i.e., classes and subclasses. There are also many multiple sub categories. The ICD code protocol has been briefly discussed above. Below is a brief overview of the CPT code protocol. It will be appreciated that the use of the ICD and CPT code protocols are used only as examples and the teachings of this disclosure will also apply equally to other code protocols.

The CPT code is another medical code set that is used to report medical, surgical, and diagnostic procedures and services to entities such as physicians, health insurance companies and accreditation organizations. CPT codes are the United State standard for how medical professionals document and report medical, surgical, radiology, laboratory, anesthesiology, and evaluation and management (E/M) services.

The five-character CPT codes are used by insurers to help determine the amount of reimbursement that a clinician or other health care entity will receive for services provided. This has been the primary function of the code protocols. The codes have not been used as a tool of treatment.

Current Procedural Terminology (CPT) codes were first published in 1966 and are developed, maintained, and copyrighted by the American Medical Association (AMA). Thousands of CPT codes are in use, and they are updated annually.

The CPT is divided into three categories of codes. Category I: Procedures that are consistent with contemporary medical practice and are widely performed. Category II: Supplementary tracking codes that can be used for performance measures. Category III: Temporary codes for emerging technology, services and procedures.

A sampling of the CPT coding scheme is as follows:
a. Codes for Evaluation and Management: 99201-99499
b. Codes for Anesthesia: 00100-01999; 99100-99150
c. Codes for Surgery:10021-69990
d. Codes for Radiology: 70010-79999
e. Codes for Pathology & Laboratory: 80047-89398
f. Codes for Medicine: 90281-99199; 99500-99607
g. Individual sections are then broken down further. For example:
h. Office/other outpatient services: 99201-99215
i. Hospital observation services: 99217-99220
j. Hospital inpatient services: 99221-99239
k. Consultations: 99241-99255*
i. Problem focused: 99241
ii. Expanded problem focused: 99243
iii. Detailed: 99244
iv. Comprehensive: 99245
v. Emergency department services: 99281-99288
vi. Critical care services: 99291-99292
Note the physician must provide enough detail to support the level of service. A problem focused visit evaluates one to five elements within a system. An expanded problem focused visit evaluates at least six elements. A detailed visit evaluates at least two elements in six systems or 12 elements in two or more systems. A comprehensive exam evaluates the entire review of systems, identifying one or two elements per system.

In addition to the CPT codes, some third-party payors require use of the Healthcare Common Procedure Coding System (HCPCS). The HCPCS incorporates two levels, i.e., the first level consists of the CPT codes. The second level identifies medical products and services not included within the CPT coding scheme. An additional coding protocol is the Diagnostic Related Grouping (DRG) codes. This coding protocol is used only to code inpatient billing claims.

As used herein, “patient electronic medical records” (or EMR) includes but is not limited to machine readable electronically stored records incorporating ICD, CPT or HCPCS code protocols. The EHR records contain the recorded text of the health care provider's observations of the patient, patient history and diagnosis, as well as treatment notes, record of ordered tests and test results. It may also contain images, e.g., X-rays and CT scans.

### Coding Function

It will be further appreciated that one function of the coding protocols or coding/classification schemes is to convert or translate the medical health care provider's (clinician) notes of diagnosis and treatment into a universally understood common “language”. Treatments and procedures performed by various providers or clinicians can be readily identified the commonly assigned treatment/procedure codes. However, currently, this common language is utilized only for billing purposes. The object of this disclosure includes expanded utilization of this common “language” to provide an index of patient specific medical diagnosis, treatment and testing index. This index can be used, in one embodiment, for searching a patient's EHR for relevant past medical diagnosis, treatment and testing.

This disclosure teaches integrating the assigned codes with the specific medical information or portion of the patient's electronic health record (EHR). In accordance with this disclosure, the EHR is annotated with the code protocols previously assigned for medical/insurance invoicing. The patient's EHR (containing past medical services, treatment, procedures, surgeries, etc.) can be searched by requesting records associated with specified codes applicable to the current medical event, i.e., illness, injury, trauma, surgery, etc.

For illustration, typing the term “kidney stone” (an example of a key word) returns one exact match under the ICD-10 protocol, i.e., “Calculus of kidney and ureter” N20. Related ICD terms are: N13 Obstructive and reflux uropathy, N21 Calculus of lower urinary tract, N42 Other and unspecified disorders of prostrate, Z84 Family history of other conditions, Z87 Personal history of other diseases and conditions.

Continuing with the illustration, entry of the key word term “knee ligament” returns approximately 20 direct CPT match entries. There are approximately another 50 partial matches. Entering the term “torn knee ligament” returns 12 direct keyword matches.

In an embodiment of this disclosure, the clinician provides medical examination services for a patient experiencing pain in his/her left knee. The examination may result in a diagnosis. This diagnosis is recorded in the clinician's record or file for the patient. Typically, this record may, or may not, contain entries of prior examination or treatment procedures. The service provide is typically relying upon the patient's memory regarding past treatment and diagnosis. In recording the instant examination for left knee pain, the clinician may notate the ICD code applicable for the diagnosis. This code is part of the patient's EHR record.

In a further example, the patient files are electronically recorded. In one embodiment subject of this disclosure, the ICD diagnosis code is part of the electronic record. The patient's electronic record may be searched for diagnoses for left knee pain by entering the ICD or CPT code. It will be appreciated that multiple codes may be entered as a search criteria wherein these multiple codes pertain to various topics or procedure related to the left knee.

The search may be broadened for other diagnoses pertaining to the left knee by entering the first three characters of the ICD code for the left knee. Note, the simple entry of “left knee” returns 2180 possible entries. For example, M25 is the code for “other joint disorder, not elsewhere classified”. This code entry is expanded by approximately 9 sub sets such as M25.01, “fistula of joint” and M25.5 “pain in joint”. Note further that the entry of right knee returns the same codes. A generic or broad entry of “right elbow pain” returns only the general code M25.5.

### Patient Encounter, Notation of Symptoms & Diagnosis and Code Searching

This disclosure includes integrating the diagnosis or treatment code(s) with the patient's electronic health record (EHR). Although EMR and EHR are sometimes used interchangeably, the EHR is deemed to have broader inclusion of records than may be contained in the electronic health record (EMR). The clinician's records may comprise a format where the ICD code (or other code) is distinguished from the text of the diagnosis to facilitate electronic search of records. The electronic search tools may include, but are not limited to, optical character recognition (OCR) components. The ICD may be separated from the text by columns, font size or font characteristic, etc.

In one embodiment of this disclosure, a CPU of a medical clinician is programed to contain one or more databases of medical treatment activity or medical diagnosis where each database contains activity or diagnosis descriptions with corresponding code designators. The CPU can contain components including but not limited to a display screen, information input device such as a mouse, keyboard, touch screen, mouse, etc., processor, and memory including non-transitory memory. The EHR is also searchable and readable by the CPU. Activity data or diagnosis data is entered into the CPU by the clinician; the CPU evaluates the data entry in comparison to the databases; the CPU matches the data entry with one or more database diagnosis or activity descriptors and corresponding diagnosis or activity database codes; the CPU records the data activity or diagnosis descriptor and one or more corresponding database codes into a patient database. The search results may be displayed to the current clinician. In an embodiment, the database may be ICD or CPT databases.

It will be appreciated that the CPU may comprise a mobile device such as a tablet computer, e.g., iPad, or a smart phone. In another embodiment, the CPU may be positioned on a cart and wheeled into the patient examination area (patient encounter) or treatment area. The CPU (i.e., “device”) display screen can be used to display various options or data entries made by the clinician. It can also display suggested code selections. The device display can also provide other options such as a search option as will be discussed below.

In another embodiment, an activity or diagnosis is entered into the CPU along with at least one corresponding ICD or CPT code.

In another embodiment, the CPU records the combined entered description of activity or diagnosis along with the database codes corresponding to the entered description.

In another embodiment, the CPU recorded data includes the patient's identity criteria. In another embodiment, the date of treatment or diagnosis is included in the recorded information within the database. The recorded data is indexed by treatment activity, diagnosis or assigned ICD or CPT code. The database may also be indexed by patient name, DOB, address or other identifier. The identifier may be one or more assigned patient identification numbers. It will be appreciated that different health care providers may assign different patient identification numbers.

It will be appreciated that a patient identifier, (e.g., patient name, patient name and DOB, health care provider identification number, SSN, etc.) may be entered into a database at the start of the patient encounter. Reference FIG. 313001. The clinician observes the patient, obtains patient history if available, examines the patient and notes symptoms and possible cause or diagnosis 3002. The disclosure teaches the clinician having a device to record this information (or recorded by a “scribe” shadowing the clinician). The device contains software that contains or has accesses to the medical codes, e.g., CPT or ICD codes 3003. The software parses the clinician's entry of symptoms and diagnosis and may present the suggested codes the clinician 3004. This information may come from a database accessed by the device. As will be discussed further, the device may have access to a database containing the patient's history of past diagnosis and treatment. It will be appreciated that the records or data may be electronically recorded health care records from multiple health care providers pertaining to the identified patient. These records may comprise the patient's EHR. In an embodiment, the clinician may initiate a search of the electronic record database. The records received from the search will be limited to the data pertaining to the entered codes. In another embodiment, the database will return records of treatment or diagnosis that related or similar to the entered search codes or search words. The patient encounter may end with the patient being discharged, conclusion of the appointment, the patient being provided medical proscriptions, transferred for testing or treatment, or the scheduling of treatment or testing.

As stated above, the ICD-10 contains over 70,000 separate available code entries. Correctly assigning a correct code for a diagnosis or treatment can be a daunting task. However, it is very important that the diagnosis or treatment of a clinician be correctly coded if the patient electronic medical record is to be useful to subsequent clinicians. Hence the value of the code suggestion function taught by this disclosure.

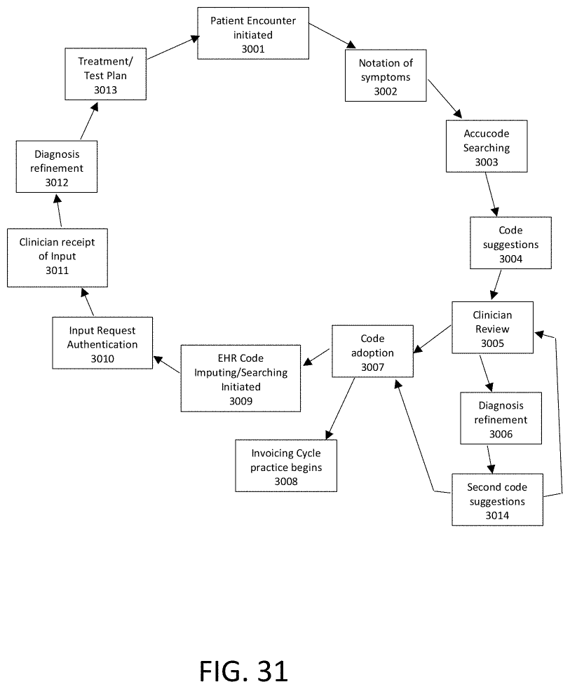

In recognition of this issue, this disclosure also includes a method for the electronic medical record to suggest possible classification codes based upon information inputted into a CPU by the clinician to for the creation of the electronic medical record. This entry of data may be concurrent with the clinician's examination or treatment of the patient. Thus the clinician receives the suggested code and descriptor (based upon the clinicians entry of data) in real time. Thus the clinician may be prompted to modify the entered terms for greater accuracy within the EHR. Reference FIG. 31 3005, 3006, 3014. Alternatively, the clinician may adopt one or more of the suggested codes 3007 which may be then integrated in the EHR. It will be appreciated that there may be multiple cycles of review before adoption. This application incorporates herein by reference U.S. Pat. No. 5,845,300 issued Dec. 1, 1998 to Ross Ward Comer et al., U.S. Pat. No. 5,978,766 issued to William W. Luciw on Nov. 2, 1999, and U.S. Pat. No. 8,670,979 issued to Thomas Robert Gruber et al. on Mar. 11, 2014. These patents are incorporated herein by reference in their entirety. These patents teach computer readable storage medium related to operating an intelligent automated assistant, i.e. artificial intelligence. The assistant can respond to speech input, i.e., speech recognition. Accordingly this disclosure includes the clinician dictating his/her evaluation or treatment and immediately receives the codes and descriptors.

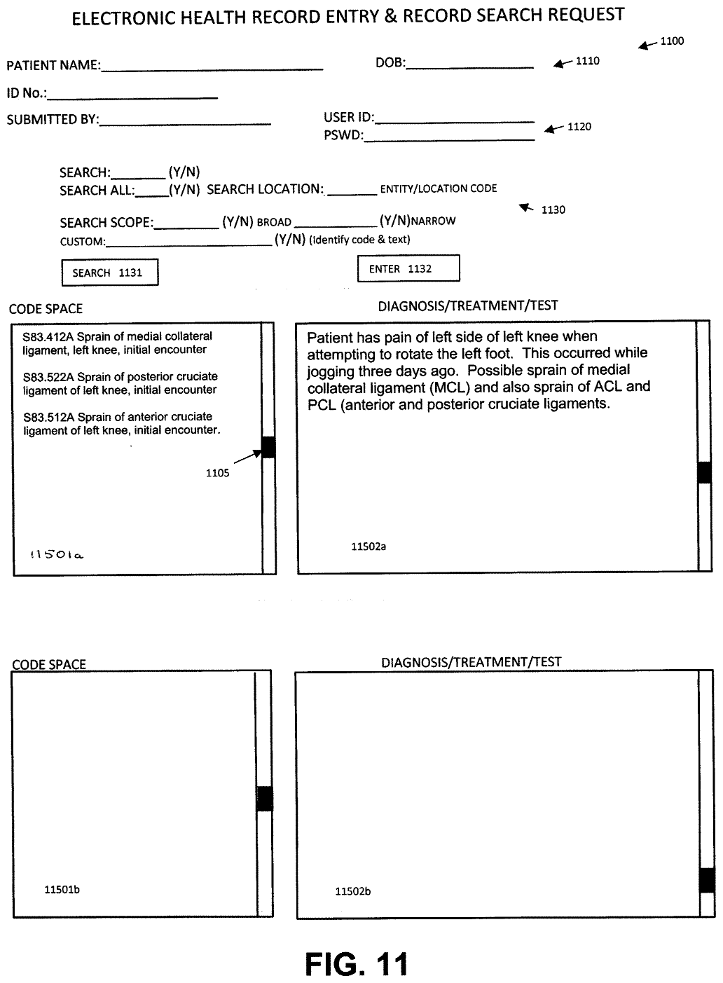

Accordingly, this disclosure teaches a system for improving the efficiency and reliability of a clinician entering data pertaining to a patient into a patient specific database or spreadsheet computer program containing medical diagnosis, treatment, procedure, tests, etc., by providing suggested entries, including but not limited to codes, (e.g. ICD, CPT) or completions to the data entry operator, e.g, clinician. The entries can include, but are not limited to, alpha-numeric, alpha or numeric codes contained in a medical provider service code protocol. The operator/clinician enters data regarding the description of diagnosis, treatment or testing into a particular data area and a dynamic list of possible codes is generated based on entered text for the diagnosis, treatment or testing, etc. The entry of text may be based upon inclusion of key words or the analysis by the software (machine learning function) of the entered text. See the suggested screen for data entry presented in FIG. 11.

Upon adoption of one or more codes by the clinician, the normal invoicing cycle may begin 3008. A search of the patient's EHR may be initiated 3009. The alphanumeric codes may be utilized in this search 3009. In an embodiment (discussed more fully below) there may be an authentication step required by EHR repositories 3010. In an embodiment, the clinician received the input 3011 from the EHR search during the progress of the patient encounter. Again, utilizing or evaluating the search results, the clinician has an opportunity to refine the initial diagnosis 3012. The patient encounter may conclude with the clinician determining a treatment or test plan for the patient 3013.

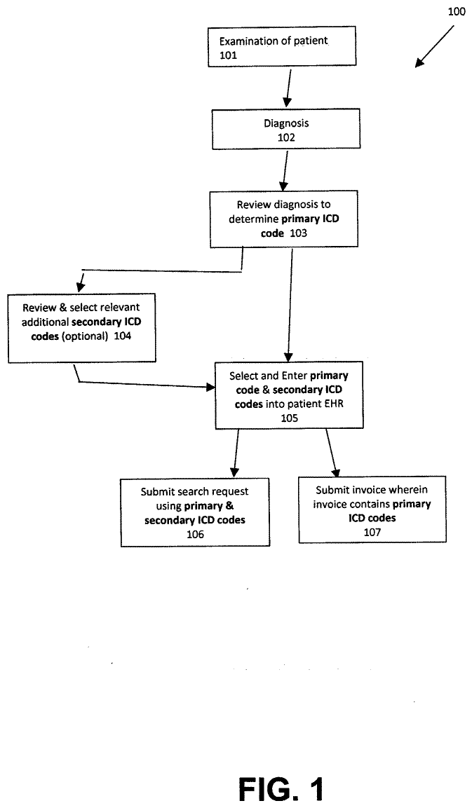

FIG. 1 illustrates an embodiment 100 of the disclosure wherein the utilization of the device and method subject of the disclosure begin with examination of a patient 101. This may be termed a “patient encounter”. Information is obtained and the clinician may enter symptoms or diagnosis 102. Suggested codes, e.g., ICD codes, may be suggested by the CPU and displayed on the device 103. These suggested codes may be the product of artificial intelligence, machine learning or key word correlation. The clinician may optionally review the suggested codes, alter the notes of symptoms or diagnosis review and select alternate or secondary codes 104. The clinician may review and select the primary and secondary codes into the patient's EHR 105. The codes therefore become part of the patient's records. The clinician may optionally initiate a search of the patient's EHR using the primary or secondary codes 106. Recall the patient may not be able to provide any or little accurate history of past health issues and treatment. The diagnosis and selected codes may be used to initiate an invoice for reimbursement of services 107. It will be appreciated that currently, this invoicing task is typically performed long after the patient encounter by “medical coders”, i.e., individuals that may not have health care training. The after the fact recording is based upon the clinician's, PA or nurse's notations of examination and treatment. Under the current practice, there are often inaccurate coding, billing errors or under billing.

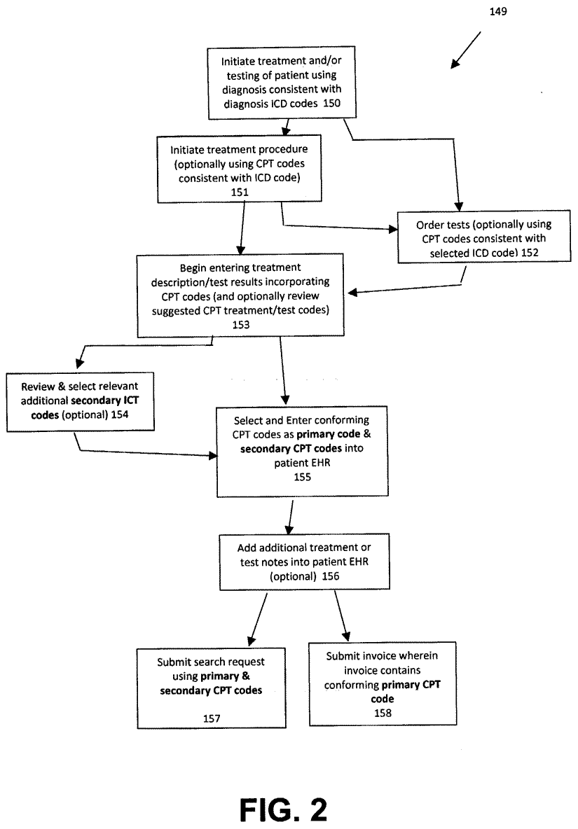

FIG. 2 illustrates another embodiment 149 wherein treatment can be commenced consistent with suggested ICD diagnosis codes 150 and correlated/consistent CPT codes 151. Testing 152 may also be initiated consistent with the clinician's adopted ICD codes and test results entered 153. The clinician may review and select additional relevant secondary codes 154. The CPT treatment codes, prompted by the device software, i.e., the codes correctly describing the treatment/test protocol selected by the clinician, are included within the patient history 155. Again it will be appreciated that this embodiment allows the correct code to be selected concurrent with treatment and testing and not “second guessed” by a later medical coder. Additional test or treatment may be conducted 156. The clinician may initiate a further search 157 of the patient's past medical history (EHR) and the clinician may enter supplemental notations for inclusion within the EHR 156. The coded information may also be utilized within an invoice 158.

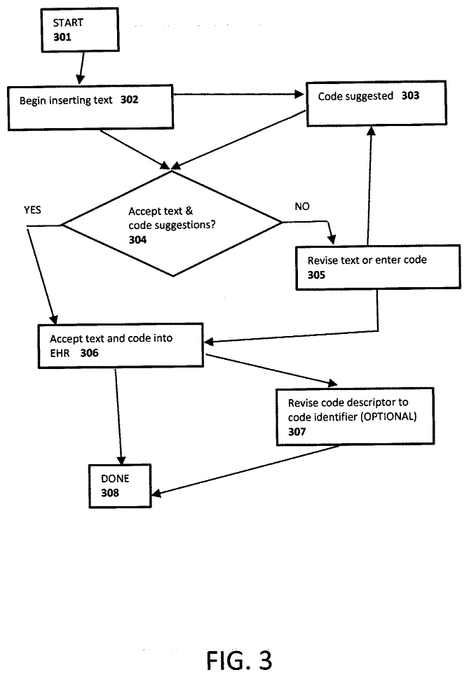

FIG. 3 illustrates a more simplified description of the disclosure. The process starts with a patient encounter where in the clinician begins inserting electronically discernable text 302 in the device subject of the disclosure. This device is described in greater detail below. The device software parses the text and suggests applicable codes. The codes, along with code descriptors, are displayed to the clinician 303. The clinician may accept the entered text and correlated code entries 304. The clinician may choose to reject the display and provide revised text 305. Alternatively, the clinician may accept the entry, select from and adopt one or more of the suggested codes and the information is included within the patient EHR 306. The clinician may optionally elect to amend the code descriptor to match the intended use of the code 307. Such practice may result in revised code descriptor's in a later up date of the code.

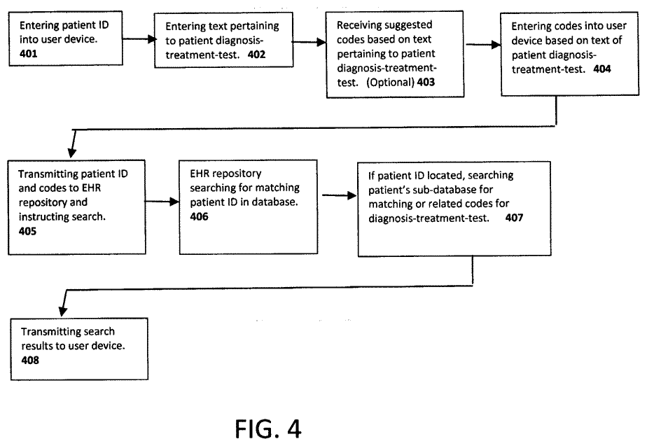

FIG. 4 presents another embodiment of the disclosure, again beginning with the patient ID information being entered or automatically presented in the clinician's device 401. This information may be obtained during the patient's initial check in at the health care entity, e.g. emergency room. The clinician enters text (electronically readable) into the device regarding patient diagnosis/treatment/test 402. Again the device software may display suggested codes pertaining to the diagnosis/treatment/tests 403. The clinician adopts the suggested codes into the text of the diagnosis/treatment/tests 404. The clinician can initiate a search utilizing the patient ID and adopted codes. 405. A search of the patient's EHR is conducted 406 and any responsive information 407 is displayed to the clinician 408. Again it will be appreciated that this process can occur within real time, i.e., seconds or minutes between initiation of the search and receipt/display of responsive information.

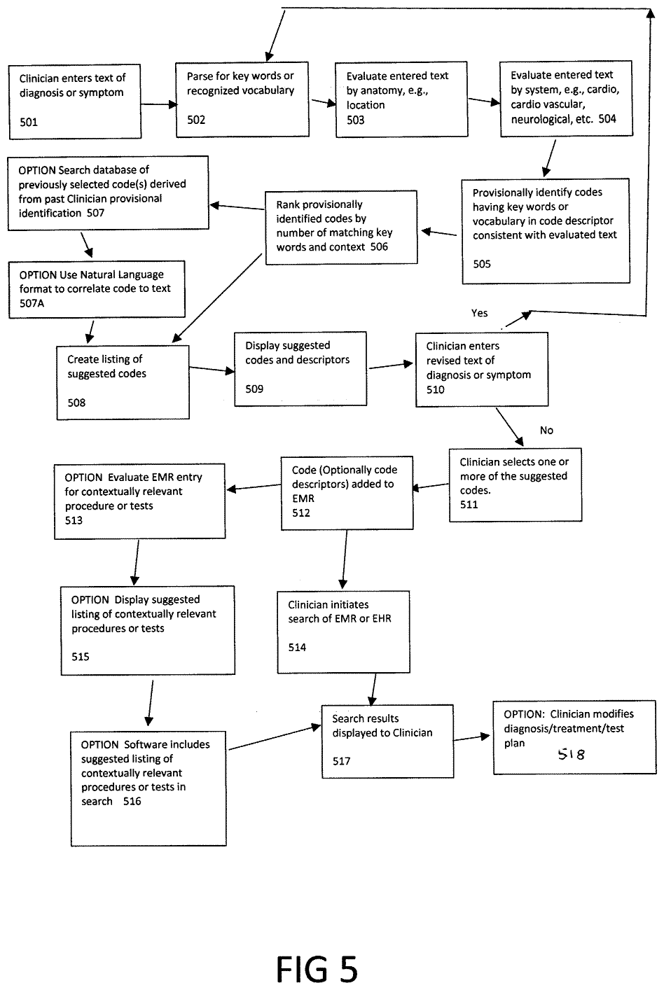

FIG. 5 illustrates a more comprehensive description of the disclosure, beginning with the clinician entering electronically readable text of the patient diagnosis or symptoms 501. The text entry is parsed for key words or recognized vocabulary 502. This can be words included with the code descriptors or which are recognized by the software as relevant to the description or context of the description. It will be appreciated that this can include machine learning. Further the software analysis includes determination of patient anatomy 503 or anatomical systems 504. This software analysis (referred as “parsing”) results in identification of one or more codes relevant to the evaluated text. It will be appreciated that in some instances the clinician's entry does not result in sufficient information to allow suggestion of relevant codes. Further, the software may rank the plurality of suggested codes by relevancy using matching key words or discerned context. 506. It will be further appreciated that the software may have the capability to search prior utilization of terms by the clinician to correlate previously adopted terms. 507. Also Natural Language format can be used to correlate codes to clinician text. 507A. In either event, the disclosure teaches developing a listing of suggested codes responsive to the clinician's text 508 and the display of such codes to the clinician utilizing the device described in this disclosure 509. At this point the clinician can review and reject the suggested codes. The clinician may then supplement her description 510. Alternatively the clinician may select one or more of the suggested codes 511. It will be appreciated that if the codes are rejected and alternate text description entered, the text analysis will be repeated. It will be further appreciated that all of these steps are electronically processed and the events are occurring in real time.

Continuing with FIG. 5, the codes are incorporated into the current EHR entry 512. As an option, the current EHR may also be supplemented with contextually relevant procedures or test (in additions to diagnosis) 513. In an option this supplemental listing may also be displayed to the clinician via the device for consideration 515. It will be appreciated that these optional steps may assist the clinician in confirming the software results are valid in terms of the clinician's notation of symptoms and diagnosis. A search can be initiated by the clinician or the patient's past EHR entries 514 and the search results displayed to the clinician 517. This search can include results from software suggested contextually relevant procedures or tests 516. The clinician may modify the diagnosis/treatment/test plan 518. Importantly, this real time search of the patient's EHR allows the clinician to evaluate her diagnosis or notation of symptoms. It will be appreciated that such opportunity is not provided to the clinician prior to this disclosure.

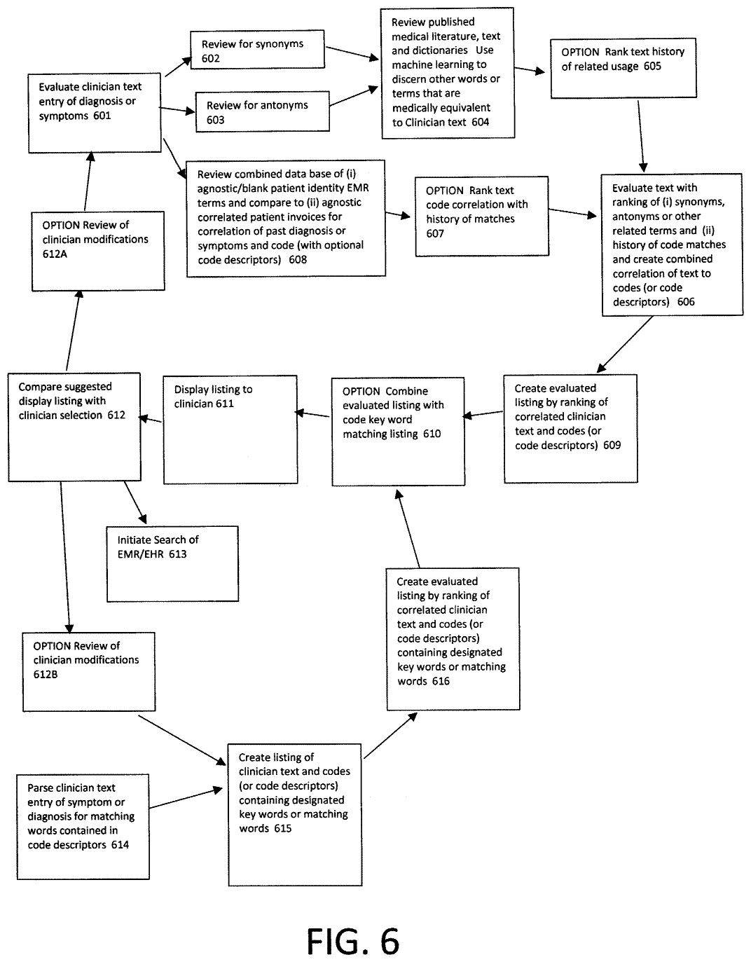

FIG. 6 is further detail of the method of the disclosure described in FIG. 5. Again the steps begin with the clinician entering an electronically discernable entry of patient diagnosis or symptoms 601. In one path, the text is parsed for synonyms 602 which can be analyzed using machine learning with medical literature 604. In another path, the text is reviewed with a combined database of previous clinician terminology with documented diagnosis or symptoms and selected codes/code descriptors 608. This review of past usage of text and correlated codes can be ranked 607. The input of these two paths can be combined 606 and utilized to create an enhanced listing of suggested codes 609. This enhanced listing can be combined 610 with a more elementary parsing of the clinician's text for words or terms matching the text of the codes and code descriptors 614. This step includes creating a listing of text and codes containing key words 615. The contents of this listing can also be ranked 616. The results of these several paths can be displayed to the clinician for adaption or modification 611. A comparison may be made between the suggested display and the clinician's selection 612 and review may be conducted 612A, 612B. The results can be used for the clinician's initiated search of the patient's EHR 613. It will be appreciated that these multiple steps and methods of analysis will achieve a highly relevant search criteria.

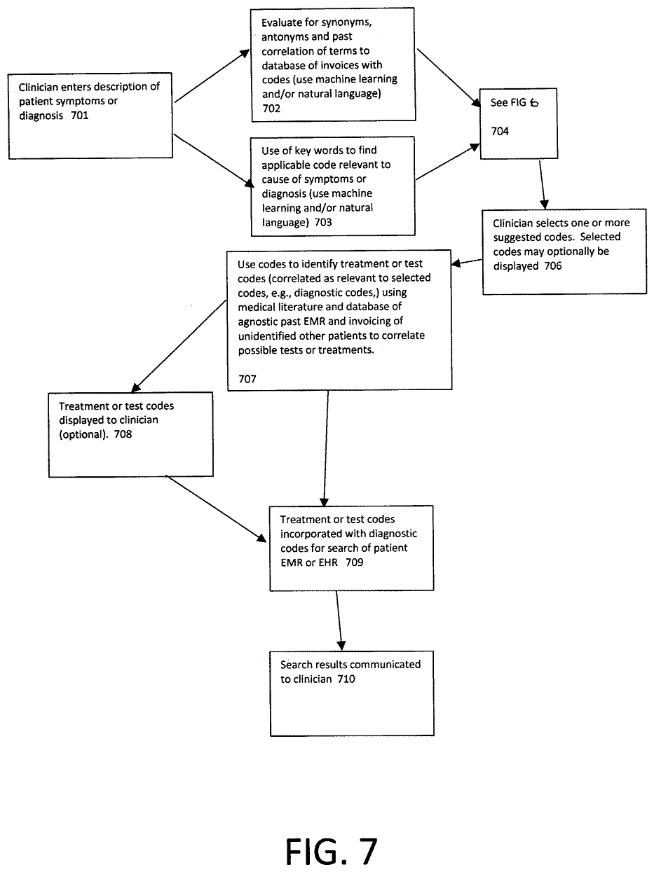

FIG. 7 expands upon FIG. 6 discussed above by utilizing the adopted symptom or diagnosis codes 702, 703 to allow the software to further identify treatment or test protocol and related codes for inclusion in the search 707. The information can be derived by access to database information of past (unidentified/patient agnostic) patient records (EMR) and invoicing. In an embodiment, this enhanced listing of treatment and tests (with clinician selection 706) can be disclosed to the clinician 708. The expanded search (including treatment and test codes) can be performed 709 and search results communicated 710 in real time to the clinician.

### Correlation and Reimbursement

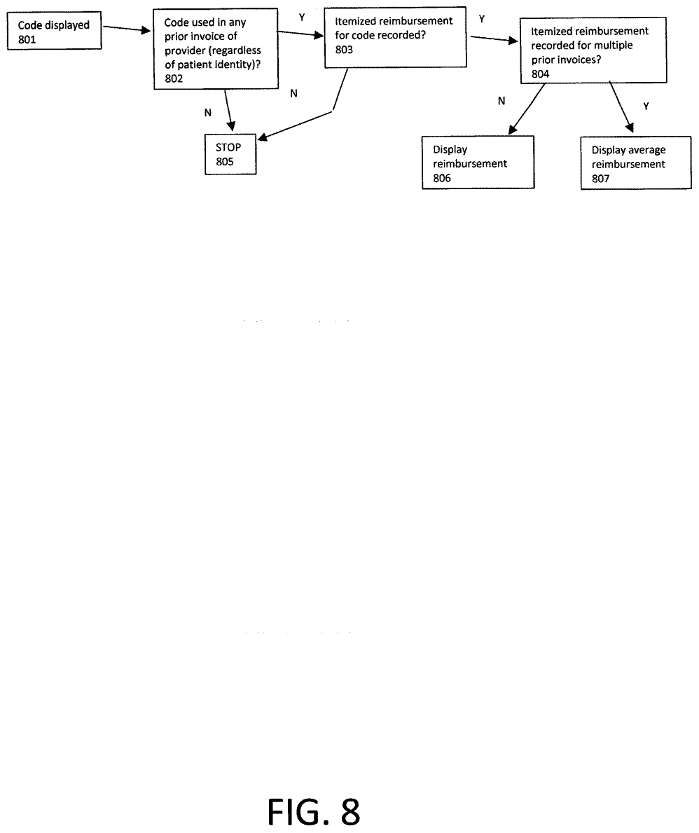

FIG. 8 illustrates yet another novel embodiment of the disclosure. The codes contained in the hospital or clinic invoicing and payment records can be are displayed 801 and correlated to past actual payment. In instances where Medicare coding (Healthcare Common Procedure Coding System (HCPCS)) is utilized, the software of the disclosure can compute the expected reimbursement based upon published schedules and modifiers for geographic regions.

Continuing with FIG. 8, the software suggested code can be correlated to an actual payment record 802 (of other patients but without disclosure of any patient identity). This review of other patient information without disclosure of patient identity is sometimes referred to as “patient agnostic”. If there is not payment history, the correlation is completed 805. If there is record of reimbursement 803, the software can determine if multiple reimbursements have been made under this code 804. In instances of multiple reimbursement, the instances may be averaged and displayed to the clinician along with the suggested alphanumeric code and descriptor 807. Individual instances can also be displayed 806.

It will of course be appreciated that all of these several embodiments for creating a search greatly enhance the clinician's ability to treat the patient, now having knowledge of the patient's history and this history having been provided within minutes of the start of the patient encounter.

The disclosure includes multiple health care providers aggregating payment rate information that can be correlated to the services described in each coding protocol. These can be payments received from similarly coded services and procedures. Some medical providers, such as hospitals with significant quantities of invoicing to various third party payors will readily have a large database of payments and reimbursements for variously coded services and procedures. Information can similarly be exchanged to clinicians through professional organizations. The professional organizations may receive this information from large entities such as major hospitals. Alternatively, health care providers may share information by payment of membership dues to professional organizations. Members may also receive access to the information as an added benefit of their EHR software program provider. Other member may receive a discount on the subscription costs for their EHR software license/subscription in exchange for providing this information.

The disclosure further includes displaying the reimbursement information correlated with suggested ICD-09, ICD-10, CPT or HCPCS codes. The information may, in one embodiment, be displayed on hand held devices such as programed iPads, tablet computers, smart phones, lap top computers, etc. utilized by the clinician at the time of patient examination or encounter.

This disclosure embodies interpretation the clinician notes of diagnoses or observed symptoms, possible treatment or test and assigning recognized codes such as but not limited to ICD-09, ICD-10, CPT or HCPCS codes to the draft clinician entries as well as displaying applicable or possible monetary re-imbursement amounts per third party payor established current rates, including but not limited to Medicare payment rates correlated to the suggested codes. This re-imbursement information, along with the displayed code descriptors, will assist the clinician in identifying one or more appropriate codes. Alternatively, it may also signal to the clinician that the entered text of diagnosis is misleading and should be revised for correct description of symptoms/diagnosis and coding into the patient's EHR.

It will be appreciated that patient payment method and existence of insurance coverage, insurance company and coverage type is routinely collected by the health care provider prior to examination by the clinician. In an embodiment of this disclosure, this information can be entered along with the patient identifying information provided to the clinician.

As the clinician makes notes of observations, diagnoses, etc., the applicable codes can be identified. The reimbursement rates for each identified code can be also identified and displayed to the clinician/user.

In one embodiment, the appropriate Medicare re-imbursement rate can be identified by operation of the disclosure by programed software implementing the following steps:
1. Selecting the Medical Professional Fee Schedule (MPFS) year of search;
2. Selecting the type of information selected from the subgroups;
(a) Pricing information
(b) Payment policy indicator
(c) Relative Value Units (RVUs)
(d) Geographic Practice Cost Index (GPCI); or
(e) All
3. Select Healthcare Common Procedure Coding System (HCPCS) Criteria;
(a) Select single HCPCS Code
(b) List of HCPCS codes (up to five codes will appear) or
(c) Range of HCPCS codes
4. Carrier/Medicare Administrative Contractor (MAC);
Note that this option will appear only if the Pricing Information, GPCI or All options may be selected in step 2 above;
   (a) National payment amount
   (b) Specific Carrier/MAC (after selecting this option the MAC must be selected)
   (c) Specific locality (after selecting this option, select locality of interest from Carrier/MA locality dropdown menu); and
   5. Click Submit to view search results.

It will be appreciated that the above listed options may be preselected by the clinician/user causing the desired search results to be automatically displayed.

Note that the step 3 criteria will not appear if the CPCI code field was selected in step 2.

Single HCPCS Code will appear at the bottom of the page

For example, a displayed CPT/HCPCs code 27381 for “repair/graft kneecap tendon” would display “Work RVUs² of 10.76 and “Total Facility” RVUs² of 23.03. For “repair of knee ligament”, the amount varies among three codes, i.e., 27405, 27407 or 27409. RVU is the acronym for “relative value units”.

In an example, the Medicare monetary reimbursement rate varies among the three above listed codes for “repair knee ligament”. See FIG. 1. For code 27405 in Houston, Tex., the reimbursement is $707.14. For 27407, the reimbursement is $832.22 and for code 27409, reimbursement is $1,013.76.

Continuing with the example of the display of Medicare reimbursement data, an additional criteria is the Geographic Area Factors or “GAF”. For Houston Tex., the GAF is 1.013. For Los Angeles-Long Beach-Anaheim (Los Angeles Cnty), California, the GAF is 1.090.

Fee payment amount (Medicare PFS Payment Rates Formula) is the sum of the [(Work RVU×Work GPCI) plus (PE RVU×PE GPCI) plus (MP RVU×MP GPCI)]×CF. The formula includes a Work RVU reflecting the relative time and intensity associate with furnishing a Medicare PFS service. Also the Practice Expense (PE) RVU reflects the cost of maintaining a practice, e.g., office rent, supplies, equipment and staff costs. The Malpractice (MP) RVU reflects the cost of malpractice insurance.

FIG. 11 illustrates the display of a device utilized by the clinician or staff during or approximate to the patient encounter. The display contains the clinician's input such as observed patient symptoms, possible limited history, preliminary diagnosis, tests to be performed (X-rays) and treatment 11502a. Suggested codes are displayed 11501a. In this embodiment the suggested codes, along with reimbursement information, may be displayed in a separate field juxta-positioned to the relevant clinician entry. It will be appreciated that other formats may be used for the display. See FIGS. 10a, 12, and 13.

In another embodiment, the information is not displayed to the clinician via a handheld device but rather on a display screen monitored by a nurse or technician shadowing the clinician during the patient encounter.

The clinician can evaluate the suggested codes with reimbursement information before or after initiating an electronic search of the patient's EHR.

### Disclosure Operation

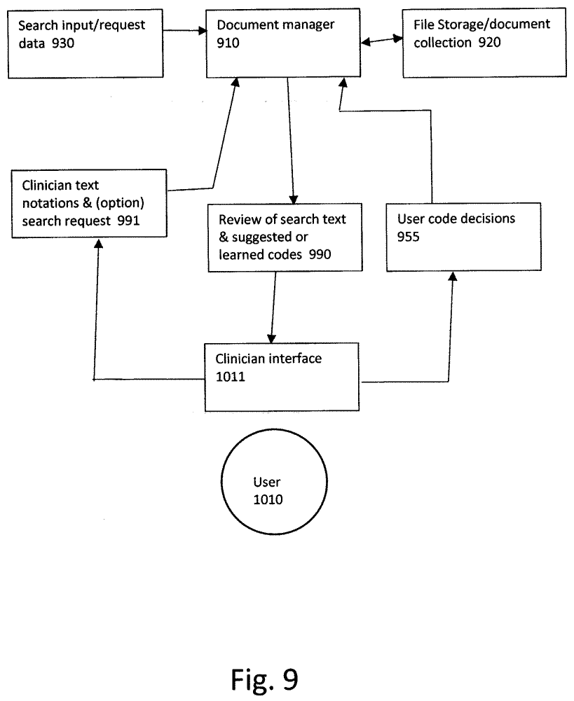

FIG. 9 further illustrates another perspective of the disclosure. The user/clinician 1010 utilizes the device (user interface) 1011 to interact with the software. The clinician's actions can be simply to record symptoms and possible diagnosis of the patient encounter. The clinician's notes may be communicated to a file storage/document collection 920 wherein past information of diagnosis and symptom terms may be accessed. Working through a document manager component 910 of the software, suggested codes (based upon matched text or machine learning) may be displayed 1011 to the clinician. The clinician may make coding decisions 955 based upon this information. Alternatively (or concurrently) the clinician may modify her notations and optionally may initiate a search through the software document manager 910 to an EHR repository or database 930, 920. The search results may be communicated through the document manager 910. The search results can be displayed to the clinician 1010 through the interface 1011.

### Autocorrect and Suggestion Functions

The classification process (creating coding classifiers or enhanced code descriptors) may begin with a first text entry. At least two predicated code classifiers are selected for the entry. The predicted code classifiers may be selected using a document information profile such as established code descriptors. The user's code selection may be used to establish a code classification.

Note that the user, i.e., health care provider, may reject each suggested code and enter a code search term as part of the ICD or CPT function, e.g., left knee ligament strain. The search term may present other suggested codes based upon the ICD or CPT code function. Alternatively, the user may alter or amend the text of the diagnosis/procedure/test entry to achieve a different selection of suggested codes.

A processing order for scoring a subset of the selected code(s) may be determined, i.e., for each of the predicted classifiers or code descriptors, a set of scores may be calculated for an EHR entry (“left knee ligament strain) using the processing order and document information profile (classifier or code descriptor) of the entry to be scored. In response to receiving a user coding decision, data that corresponds with the received user coding decision may be identified (e.g., a set of scores calculated for a predicted classifier).

As described, the system facilitates the assignment of these indexing codes by the health care provider/clinician. This may reduce the time and expense of separate individuals deciphering the clinician's notes and then assigning codes. It will be appreciated that the ICD code protocol contains approximately 78,000 separate codes. The system subject of this disclosure may display suggested codes to the clinician at the time the clinician is making his/her entry into the patient's electronic file.

Significantly, utilizing machine learning as part of artificial intelligence, the system may learn to assemble more accurate sets of suggested codes in response to the clinician's text entry. This learning may be based upon prior text entries, search results, medical literature and selected codes. The learning could be based not only upon the suggested codes that are selected by the clinician, but the final billing codes entered for the patient's health care event. The goal is to create a common vocabulary and definition of terms between the clinician and the code descriptors of a code protocol.

Therefore, the system/algorithm may learn from a clinician's text descriptions and past selected codes, i.e., the set of text terms that lead to a coding selection. The code description (in combination with the code descriptor) can be either expanded or narrowed, i.e., the original code descriptor can be amended to result in suggestion of a code correlated to the clinician's text entry within the EHR. The clinician will not have to amend his/her vocabulary to match the code descriptors of the code protocol.

The system can utilize an initial clinician selection of text or word entries that may be broader than the code descriptors of the ACD or CPT protocols.

The existing protocol or expanded protocol can be used as key words for selection of codes (coding decisions) by the clinician.

Further, the system (machine learning algorithm) can extract information profiles and themes from the clinician's text entry (i) created as part of the diagnosis step, (ii) from the treatment procedure and progression or (iii) from evaluation of test results. The system may also use accepted descriptions of treatment procedure (such as definitional terms or phrase of treatment/procedures such as an appendectomy) or the text and range of test protocols. This learning function can discern test results outside accepted ranges and classify or define the resulting test condition (e.g., hypoglycemic).

Also, the system may develop identifiers or classifiers that result in suggestion of one or more codes, i.e., use of the identifiers. These identifiers may be deemed to be enhancements to the code descriptors published with the original code protocol. It will be appreciated that code descriptors are established by the authors of the established codes. For example, the CPT is a copyrighted property and creation of the AMA. As already stated, the code and code descriptors are a tremendous resource with applications and value far beyond merely processing and classifying invoices for medical services.

The extraction of information to develop amended identifiers for the code should increase the accuracy of the suggested codes to the clinician's descriptive text entry for a diagnosis, etc.

It will be appreciated that suggestion of codes for a single patient entry, including the development of identifiers that will be used by the system, is repeated for all other patients. The system thereby is continuously learning and refining the code identifiers beyond simply matching the code descriptors to clinician text entries. This is active learning.

Further, the system algorithm may rank the suggested codes by how well the text, etc., fits with the code identifiers. As stated above, the code identifiers are enhancements to the descriptors of the code protocols. The ranking may be by the number of letters within the text that match the code descriptor. Alternatively, the ranking may be based on whether there is a text match of a noun versus adjective of the noun. A match of nouns between the code descriptor and text entry may rank higher that a match of adjectives.

### Device & Screen Display

As already stated, the software of the disclosure may be used with mobile electronic devices have user input capability, non-transitory memory, processor, data transmitter and receiver, and display functions. Such devices can be tablet computers, e.g., iPads, smart phone, laptops or CPUs.

Referencing FIGS. 10a-10f, a patient specific identification is displayed 1003 matching the patient subject of the encounter. In one embodiment 1000 suggested (or selected) codes may appear within the entered text or in a separate designated area of a device. For example, the data entry field 1002a may be accompanied by a column, row or machine fillable block (“code space”) 1001a located to the left or right of the data entry field. The code space may be designated as “code”, or “diagnosis, treatment, test code” or similar. As the clinician enters text into the data field 1002a, possible codes may appear in the designated code space 1001a.

The disclosure can include an algorithm that suggests text entries in response to data, e.g., text, entered by the health care provider. This can be substitution of words, i.e., “ligament” in place of “liagament”. The algorithm may suggest “knee” in response to the partial text entry “knie”.

In an embodiment, the disclosure includes an algorithm that will suggest ICD, CPT, etc., codes in response to an entry “knee ligament” or “torn knee ligament” in contrast to entries for “sprained knee ligament”. The suggestion may include, but is not limited to, the five digit code but also a code description. For example one possible ICD code suggestion in response to the “torn knee ligament” entry could be:
a. “S83.20 Tear of unspecified meniscus, current injury”
b. “S83.209 Unspecified tear of unspecified meniscus, current injury, unspecified.”
c. “S83.209A Unspecified tear of unspecified meniscus, current injury, unspecified knee, initial encounter.”
d. “M23.20 Derangement of unspecified meniscus due to old tear or injury”

Sample CPT code examples:
a. “27405 Repair primary torn ligament/capsule knee collateral”
b. “27407 Repair primary torn ligament/capsule knee cruciate”
c. “27409 Collateral and cruciate ligaments”

See for example FIG. 10a. Illustrated is an embodiment of a screen display. The clinician may fill in the patient identifying information 1003. It will be appreciated that this information may be entered by a supporting clerical person, nurse or otherwise. The clinician may proceed to enter the diagnosis, the treatment as applicable as well as either tests ordered or test results. This information can be entered in one or more text blocks 1002a, 1002b, etc. The applicable codes may be entered in the code spaces 1001a, 1001b, etc. In a preferred embodiment, the suggested codes may populate the code spaces as the clinician types or enters text in the text block. The suggested code may be in reverse video (designated herein by the brackets [ ]). This auto insertion of suggested codes is a function of the data entry program as described more fully below. The clinician can accept the one or more of the suggested codes or insert the clinician's own selection. FIG. 10a shows a sample diagnosis 1002a and concurrent display of ICD codes applicable to the diagnosis 1001a. The codes and descriptors are added automatically by the software algorithms subject of this disclosure.

Referencing FIG. 10b and continuing with the example, the health care provider/clinician could enter a test or treatment in text space 1002b. The clinician determines to X-ray the knee. This is entered in text space 1002b. The disclosure algorithm suggests three alternate X-ray tests. The CPT codes and descriptors are displayed in the code space 1001a. The suggested text is in reverse video, i.e., here initialized and contained in brackets [ ]. The clinician can select one of the suggestions. FIGS. 10a and 10b illustrate one embodiment as to the display format and sequence of diagnosis/treatment/test text and the suggested code(s).

FIGS. 10c, 10d and 10e illustrate auto fill features of the software. Partial entries of terms may be completed by the software using machine learning. It will be appreciated that this feature will encourage use by clinicians, pressed for time, etc. Illustrated is completion of the word “ligament” based upon partial entry. It will be further appreciated that the software also continues to select codes applicable to the presumed term intended by the clinician, thereby saving time.

The text blocks 1002a, 1002b may also utilize an auto fill feature wherein entering “knee” in the text space, e.g., 1002b, may result in the appearance of one or more suggestions such as “knee inflammation”, “knee ligaments”, “knee meniscus”, etc. These suggestions may also appear in reverse code. The user may select one of the selected descriptions for diagnosis or treatment. It will be appreciated that the suggested applicable code(s) for each suggested expanded description may simultaneously appear in the code space in reverse video. Further, it will be appreciated that upon selection of the expanded or alternate diagnosis or treatment description, the code and descriptor will appear in the code space, e.g., 1001a. In one embodiment, the suggested code will be displayed in reverse code for acceptance by the clinician.

The suggested code selection may be based upon text entered by the clinician. It may also be based upon the clinician's past use of terms. As the clinician enters characters of text data item, the list of corresponding diagnosis, treatment or test codes are searched for an entry that is appropriate for or matches the entered data item. If a code match is found, then the matching code (or codes) is displayed in the code space to the clinician as a suggested completion. The clinician can then elect to accept the suggested completion or to continue entering the data item.

For example entering the term “torn ligament” may trigger the display of CPT (or similar) code to appear in the code space. Depending upon the specificity of the entered text, multiple suggested codes may appear. See FIG. 10f. In the embodiment, the disclosure algorithm displays alternate suggested codes (in reverse video). In response, the clinician may add descriptive text to the description of the diagnosis/treatment or test appearing in the text block 1002b. Alternatively, the clinician may select one of the suggested codes appearing in code space 1001a. In an embodiment, the selected text and code may move to the diagnosis/Treatment/Test block 1002a as part of the incorporation or adaptation step of placing the codes in the EHR. In another embodiment, the algorithm of the disclosure presents additional text (in reverse video) to provide the location, etc., of the ligament. The clinician may complete the text data entry and accept the final code or codes appearing in the designated code space. Alternatively, the clinician may select a desired code when it appears and complete the entry of text.

In another example, the code space 1001a may be automatically populated upon entry of pre-selected text in the text space 1002a. This example may apply when there are only limited codes applicable to the services provided by the health care provider.

This aspect of the disclosure frees the clinician from attempting to memorize the applicable codes or search a code index to select the appropriate code. This accelerates the data entry process and provides greater accuracy in entering the correct code for the services, treatment or diagnosis, as well as entering of the description of service or treatment.

As suggest above, in another embodiment, the algorithm may suggest treatment or test and related codes in response to the clinician's symptom or diagnosis entry. This will provide a more complete or robust search, again casting a wider net for a search of the patient's EHR history.

The display may contain a “Search” button to initiate a search of the patient's EHR. It may also include a button to reject the suggested code. Acceptance of the suggested code may cause the entry to replace the health care provider's notes or to be included within the accepted/integrated code protocol entered into the EHR.

FIG. 11 illustrates a further embodiment of the display screen 1100. The patient information is entered and displayed 1110. Also is the identity of the clinician user 1120. This information may be utilized as part of the authentication steps discussed below. Optionally, the screen display may provide input 1130 regarding the scope of any initiated search. Again, this may be a decision made by the clinician in the interest and urgency of time. The display provides for a “one click” initiation of a search 1131. Also there is the one click initiation to accept entered and suggested data into the device (and EHR) 1132.

In regard to the clinician being able to select a “broad” or “narrow” search is the goal that the suggested codes include the generic code as well as the species that may be selected by the clinician (or modified by amendment of the code descriptor). For example, a code indexed diagnosis for pain in knee may include broad generic codes for knee as well as one or more species codes for specific to the clinician's diagnosis. The device subject of this disclosure may allow the health care provider to tailor a search broadly or narrowly. For example, a diagnosis of pain in the patient's right knee may include: “M25.561 Pain in right knee”. However, in one embodiment, the clinician may request a broader search, to include the code: “M25.56 Pain in knee”.

FIG. 11 also illustrates are “scroll bars” 1105 or scrolling features (controlled by finger “swiping”) to allow greater information content to be accessible to user via the screen. In an embodiment, the clinician may elect to have displayed only segments of information such as code space 1101a or Diagnosis/Treatment/Test 1102a. Again, this selection feature may be controlled by “swiping”. Also it will be appreciated that this feature may be most useful when a mobile device, having limited display screen space, is being utilized.

In another example, and referencing FIG. 11 discussed below, the clinician may enter the class descriptor ICD-10-CM S83 Dislocation and sprain of joints and ligaments of knee. This class descriptor correlates to the suggested subclasses S83.412A, S83.522A and S83.512A listed in FIG. 11.

In yet another example, the clinician may elect broad search to include the following ICD-10 codes:
i. M17 Osteoarthritis of knee
ii. M24.56 Contracture, knee
iii. M24.66 Ankylosis, knee
iv. M25.16 Fistula, knee
v. M25.46 Effusion, knee
vi. M25.76 Osteophyte, knee
vii. M67.46 Ganglion, knee
viii. M94.26 Chondromalacia, knee

These codes are directed to distinct anatomical structures of the knee as selected by the clinician.

In another embodiment, the clinician input device may allow the clinician to select the scope of the search from “narrow” to “broad”, thereby automatically adjusting the scope of the codes and code descriptors (stored in the device memory) to be included in the search of prior patient EHR records. In yet another embodiment, the clinician may select among the above 8 listed categories of ICD-10 codes.

In yet another embodiment, the clinician may request that the listed medical code entries of an ICD-10 or CPT code protocol be correlated to include similar code entries of at least one of the following alternate codes protocols, Healthcare Common Procedure Coding System (HCPCS), Diagnostic Related Grouping (DRG) codes, etc.

Using the identified data, i.e., the EHR entry, the code and the code classifier/descriptor, the text of the EHR entry may be classified by the algorithm of the system into one or more of the classes or subclasses. It will be appreciated that this will enhance the returns in subsequent searches.

### Machine Learning

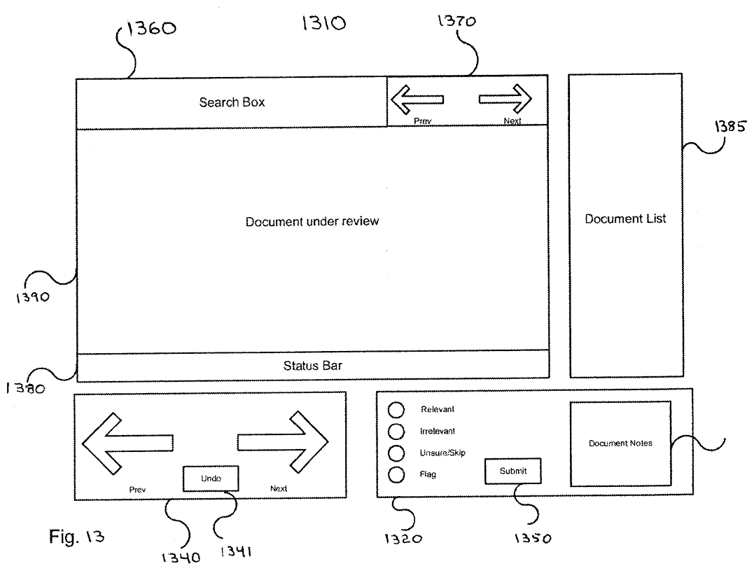

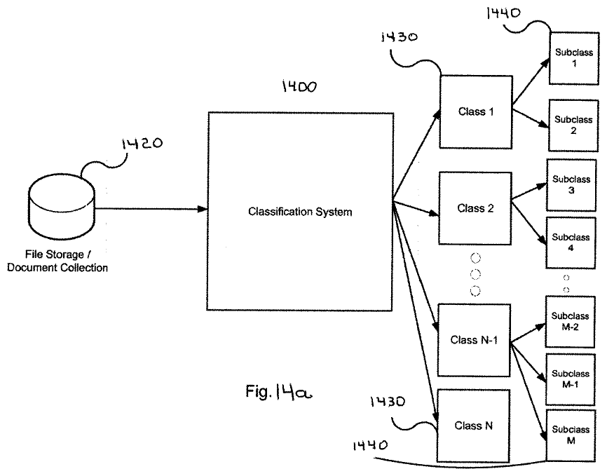

The codes, along with the code descriptors, are established in the code protocol. The EHR entries (frequently termed herein as “diagnosis/treatment/tests) can be classified or indexed using the code descriptors. The active learning algorithm may classify the EHR entries by a process that may be termed “forking”, i.e., creating a number of classification paths corresponding to predicted user's (i.e., clinician's) coding decisions. For example, multiple statements can be created in the form “if X then y”. (And if not, then Y2 is implicit.) Such a statement divides (classifies) data into two classes, here y and Y2. This is an example of forking. See FIG. 14b discussed infra. Further, the active learning algorithm may determine an order in which individual EHR entries of the EHR patient record may be processed and scored utilizing the forked classification paths. The use of predicted classifiers and forking allows the classification system to take advantage of the latency in review of EHR entries by a clinician. Forking allows the classification system to have a least a set of partial calculations ready by the time of the first clinician's coding decision. These partial results allow the classification system to suggest codes in for a next similar text entry in an interactive and real time manner, thereby speeding up the coding process.

Other statistical models may be utilized in performing this machine learning function. Examples include maximum entropy modeling, support vector machines and conditional random fields, among others.

The collection of created code classifiers may be the composition of multiple EHR records pertaining to multiple patients. It will be appreciated, however, that information contained in one patient's EHR is not transferred or disclosed in relation to another patient. Rather, this is a process initiating and continuing the active learning of the system algorithm. The algorithm utilizes past assignments of codes to past clinician entries of symptoms and diagnoses. This allows the machine learning function to recognize and suggest codes for various text entries used to describe the same symptoms or diagnoses. This facilitates assignment of codes to clinician “free text” entries. In addition, a processing time may be allocated for calculating the set of scores corresponding to a subset of selected codes.

In one embodiment, the system algorithm is additionally capable of receiving or providing relevance rankings to a subset of selected codes. The ranking process may be derived from the clinician's selection of codes. For example, relevance rankings may be generated by (a) one or more keyword searching algorithms or by (b) a comparison with one or more exemplary documents (e.g., documents from or outside of the collection such as teaching EHR entries). Additionally, a (c) processing order for the documents to be scored may be derived using the identified data and relevance rankings. A document may be selected by choosing between rankings generated from the identified data, relevance rankings or a combination of the identified data and the relevance rankings. Furthermore, systems, methods, and computer readable media are capable of managing priority queues for ranking the documents of the document collection. Priority queues may be ranked or ordered using identified data and/or relevance rankings.

For example, a “one-off” clinician text entry for a symptom or diagnosis that is ultimately assigned a code will have a low ranking for selection of a future suggestion of that code.

The coding classifiers recognized by the algorithm may be updated or amended based upon the selection history of text used by clinicians in past EHR record entries. This is a function of machine learning of the code selection made by at least one health care provider corresponding the provider's text entries or data entries pertaining to a coded patient diagnosis/treatment/test. The Systems, methods, and computer readable media are also capable of updating a code identifier (amended code descriptor) based on the clinician's selection of a suggested code in relation to the clinician's entered text and a learning rate for limiting the effect of the single code selection and corresponding EHR text. This machine learning may be useful in creating future updates of coding (such a future ICD-12 code protocol).

It will be appreciated that there exists a pattern between relevant words of the code descriptors/identifiers and the text entries. The text “knee” will statistically have a set of adjectives, e.g., “sprain”, “torn”, “strained”, “pain”, etc. Also the code identifiers will be created based upon the clinician's selection of codes (and the code descriptors) for multiple EHR text entries. It will be appreciated that the text entries may use a different but related vocabulary to describe the same patient condition. Finally, it is difficult to formulate a mathematical expression showing the relationship between text entries and possible code identifiers. It is therefore useful to employ a mapping function utilizing a machine learning algorithm that maps a relationship between the input data, i.e., text entries and the output data, i.e., elected codes. The utilization of the mapping function can be said to achieve an approximated target function. The approximation may utilize one or more assumptions and the assumptions may be refined over the course of evaluating multiple input data, i.e., entries of the health care provider entered into multiple EHRs. The fact that a large number of entries may be made in performance of the diagnosis/treatment/test activities justifies valuable use of machine learning.

It will be appreciated that an alternate but related method to machine learning would be studying the relationship of the input data, thereby creating a statistical inference. It will also be appreciated that the machine learning continues with the creation of each EHR text entry and selection of applicable codes.

In yet another alternative, it may be possible to assign numerical identifiers to set of text words, e.g., knee, elbow, femur, tibia, pelvis, stomach, lung, etc. Also, numeric identifiers could be associated with adjectives, e.g., swollen, edema, enlarged, engorged, sprain, torn, sore, strained, inflamed, etc. The combined numeric variables could be iteratively refined over a set of EHR text entries evaluated and the numeric variables of selected code identifiers (output variables) can be expressed as a combination (weighted sum) of the set of numeric input variables.

Additionally, systems, methods and computer readable media are disclosed for implementing an embodiment of the disclosed system as a user-friendly, disposable, integrated, portable, turnkey solution. For example, the above described classification tool may be implemented on single a device (e.g., a standard PC computer, tablet, laptop, smartphone, or other device) utilized by a clinician. Such devices are elsewhere referred to a client device or user device. Such a device may run a standard operating system (e.g., Windows, Linux, OSX, Android, iOS) and the classification system is conventionally installed as one or more programs or libraries on the device itself. When the device is, for example, a laptop, tablet, or smartphone, the classification system is easily transportable. Such a device may or may not be further connected to one or more computers or other devices via a network or it may be connected via a WiFi system. Alternatively, the classification system may be contained on computer readable media (e.g., a CD, hard disk, USB drive, and/or other bootable media) which, when inserted or coupled to the device, causes the classification system to be run entirely on the device. Such a device may or may not be further connected to one or more computers or other devices via a network.

The above systems, methods and media therefore increase the overall effectiveness of the classification effort by improving both precision and recall, while requiring less computational resources (e.g., workstations, servers, and infrastructure), and less manpower to initiate, maintain and oversee document classification. Moreover, in certain embodiments, the speed of the classification effort is improved through the use of classification vectors rather than matrices, with further improvements realized by reducing the dimensionality of the vector space and/or by using binary values to represent the vector elements.

This disclosure includes providing multiple suggested codes based upon a machine evaluation and learning of the word content of the clinician's text EHR entry to the word content of the code descriptor.

The system algorithm provides enhanced classification EHR entries created by clinicians. The algorithm improves upon the set of suggested health care codes (for example ACD and CPT code protocols) that can be presented/suggested to the clinician when creating an electronic record of a patient diagnosis, etc.

It is a goal of the disclosure to provide an improved method of assigning specific alphanumeric code entries to individual text entries created by the clinician. The disclosure includes presenting suggested code entries to the clinician as the entries are created based upon matching the subject matter of the text entries with code classifiers that have be assigned by the creators of the code protocols. Examples of code protocols include ICD-10 and CPT. Also, for example, multiple codes contained in a code protocol may be relevant to an entry of “left knee strain”. It will be appreciated that in each code protocol, every alphanumeric code is accompanied by a relatively brief description, hereinafter termed a “code descriptor”.

Further, this disclosure includes incorporating the relevant/applicable code or codes into or indexed with the clinician's individual text entry. The code becomes part of the patient's EHR. The EHR may be searched by identifying the patient and inputting the assigned code or codes. Past EHR entries may be searched contemporaneously with the clinician's examination of a patient and creation of a diagnosis. The diagnosis will also incorporate or be indexed by the code.

It will be appreciated that inasmuch as the EHR records are electronic, the EHR should be readily searchable and requested past EHR entries pertaining to the furnished codes can be promptly returned to the requesting clinician.

### EHR Search Request—Search Request Initiation

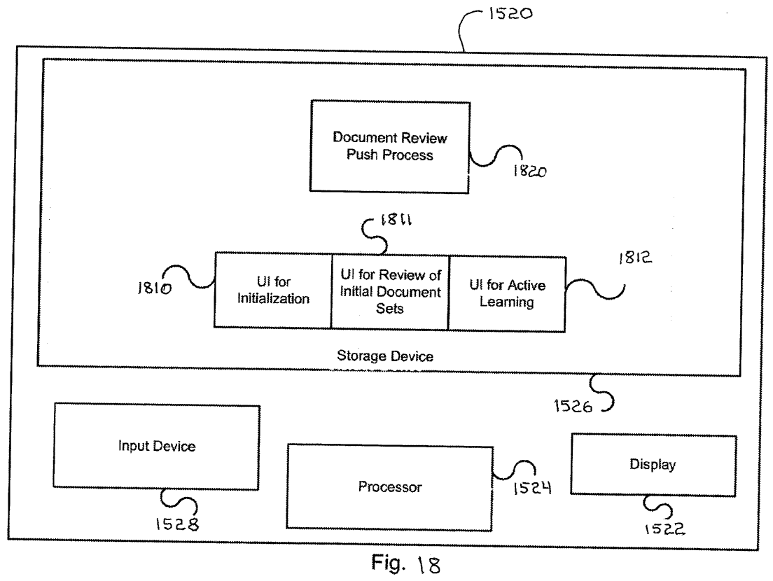

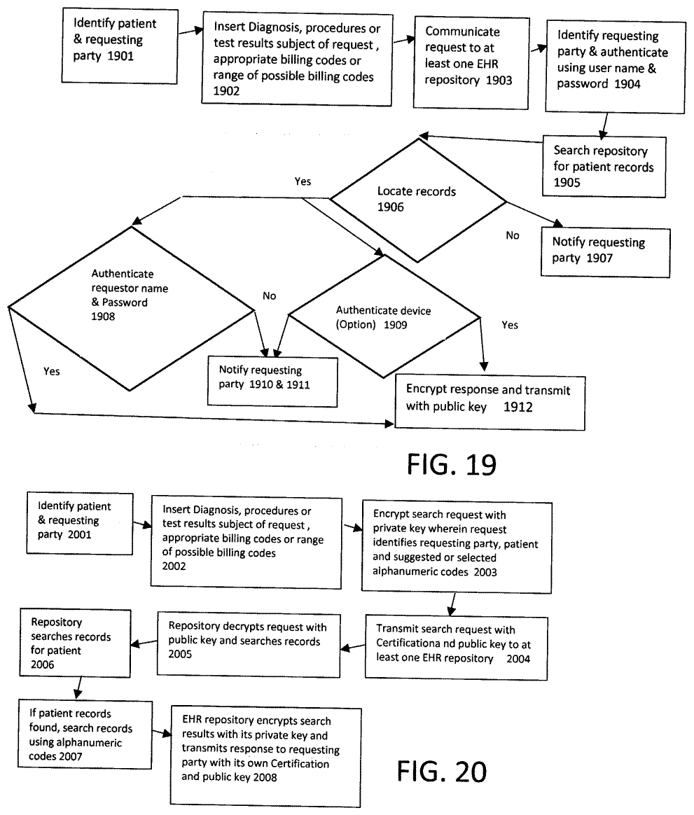

FIG. 11 illustrates another embodiment of the device and particularly the display screen 1100. The request for a record search may be activated by entering the information prompted on the display screen 1100 indicated on the data entry form (FIG. 10a) and pressing the search key 1131 of the user input component 1120. In other embodiments, one or more radio buttons may be used. As in FIGS. 10a-10f, data can be entered into an input space 1002a, 1002b of the display with suggested codes appearing in code space 1001a, 1001b. The patient identification information may be entered 1110. Also the user (clinician) entering the data may be identified 1120. The user may be subject to pre authorization or identification. This authorization or identification standards may be subject of procedures as discussed in relation to FIGS. 19 and 20 infra.

Note that the device illustrated in FIG. 11 allows the user to initiate a search 1131 of the patient specific EHR. The scope of the search may be specified to be broad 1130, wherein perhaps all related search codes or search words may be use as supplemented by machine learning or the search be customized or limited to certain key phrases or codes. The contents of the display screen may be entered or integrated into the patient specific EHR by activation of the “enter” key 1132.

Note further that in one embodiment of the device 1100, each diagnosis/treatment/test data entry space 1102a, 1102b and each code space 1101a, 1101b contains a scroll bar 1105 or similar device that allow entry and view of content within a space larger than the specific size of the screen display, i.e., 1102a.

FIG. 13 illustrates another embodiment of the device display screen 1310 wherein search result may be displayed and reviewed utilizing a portion 1390 of the display screen. The device has a scroll function 1340 allows searching of multiple data entries of the screen 1390. Note further that data entries may be designated by clinician's estimation of relevance or accuracy 1320. Relevant data may be selected by the clinician and submitted 1350 into the EHR. An overview of responsive EHR data (search results) 1385 may be displayed. The products of the search may also be reviewed in the display section 1390. This review may use the scroll arrows 1340.

It will be appreciated that the user may, after review of initial search results, modify the code entries or diagnosis notations to conduct a follow-on search. This follow-on may search, if initiated within a specified time, may avoid the clinician from re-initiating the authentication procedures or “log-in” as described infra.

The current clinician can access the patient's EHR by several methods. The clinician can create a search request, identifying the requestor, the patient name DOB, social security number, and if applicable, a hospital assigned ID number or similar. There should be include some type of security such as encryption to assure patient confidentiality is not breached. See the discussion below.

Also, the Health Insurance Portability and Accountability Act (HIPAA) does not require a patient's consent be obtained by a health care provider for disclosure of records for continuing medical treatment. Therefore consent should not be required to allow the entity having custody of the past EHR records to furnish requested information to the current health care provider. However, the requesting clinician may want to confirm that the information is being requested for the purpose of furnishing health care services to the patient. (It will be appreciated that specific authorization is required by HIPAA to be obtained from the patient to disclose the information for purposes other than treatment, billing or insurance purposes.)

A further goal is to minimize the time and effort required of a current clinician to obtain the past diagnosis/treatment/test EHR records for a patient relative to the current diagnosis (or for the process of establishing or confirming a diagnosis or treatment plan). This may be accomplished in several ways, including but not limited to the current clinician being provided an option to search within a patient's EHR of past medical events during the diagnosing process. It will be appreciated that the patient may not be able to identify past health care providers, e.g., hospitals or clinics, or provide reliable dates as to when the past treatment was furnished. Also the current clinician may search within the patient's EHR for a past record of symptoms or conditions now observed in the patient. The clinician may enter text of observed symptoms/conditions.

In an embodiment of the disclosure, the clinician may enter codes applicable to allergies to medications. In yet another embodiment, the clinician could enter codes applicable to specific existing medical treatments or medications such as cardiac stents, valves or other items such as treatment with blood thinners. These could be a regimen of codes that could be proactively searched at the start of the patient encounter. Also the patient's allergy history can be searched along with the patient's current regimen of medications.

The clinician may receive suggested codes and code descriptors from the system subject of this disclosure. The suggestions arise from machine learning as well as word searches of the text in real time. The clinician may continue with entering text, modifying text in response to the prompted code/code descriptors or selecting at least one of the prompted codes. In one embodiment, the clinician may be provided with the option to search for the patient's EHR and to request information relevant or responsive to the prompted codes.

### Word Search

In regard to word searches, an embodiment of the disclosure comprises a multi-tiered software application designed to facilitate the use of ICD, CPT codes, etc., in patient diagnoses and procedures. It is provided in web-based mobile and tablet formats, as well as desktop format. Note the embodiment and algorithms described below reference the ICD code protocol. However this description is also applicable to the other code protocols discussed elsewhere in this disclosure.

Several different methods will be used to allow the clinician to pinpoint what code(s) to apply to the patient encounter for enhanced diagnosis and real-time EHR record searching:
Medical Term Match—A match/find of ICD Codes by a mapping of those medical terms (as keywords) that are found in the description of an ICD Code.
Patient Encounter Synonyms (current)—Code matching through keyword extraction (current encounter)
Patient Encounter Synonyms (previous)—Code matching through keyword extraction (previous encounter(s))
Entity-wide Encounter Synonyms—Code matching through keyword extraction of a table of codes from encounters held across the system, not just those from the specific patient's encounters. It will be appreciated that this step creates more robust search since the disclosure is making the query of what codes have been assigned to similar diagnoses or medical conditions, i.e., patient agnostic.
Invoice Match—Code matching against code used in previous invoices of the current patient. (Has the patient been treated for this or similar condition in the past as determined from the invoice records? Again this creates a more robust search.)
EHR Match—Code matching in previous Electronic Health Records (or electronic medical records, i.e., EMR) of a patient. This can use input from the Invoice Match. This can be useful when the code protocols have not be incorporated in past EHR entries.

### Core Tables

In an embodiment, there are a core set of tables used for operation of the disclosure:
ICD Code lookup table, Diagnosis Codes—Stores a list of each ICD diagnosis code, its short description, and long description.
ICD Code Lookup table, Procedure Codes—Stores a list of each ICD procedure code, its short description, and long description.
Medical Term lookup table—Store a list of medical terms, the medical term and its associated description.
ICD Code/Medical Term mapping table—Maps ICD Codes to medical terms based on the match of the medical term as a key word in the long description of the ICD Code.
Encounter/ICD Code table—Stores a record of each ICD Code match per encounter; contains the clinician's notes that are used to extract keywords using the Key Extraction. Also the table stores a record of each code match per encounter.

### Medical Term Match

### Data Preparation.

Both the Patient Encounter Synonyms (current) and Patient Encounter Synonyms (previous) methods utilize a table of medical terms in the matching process.

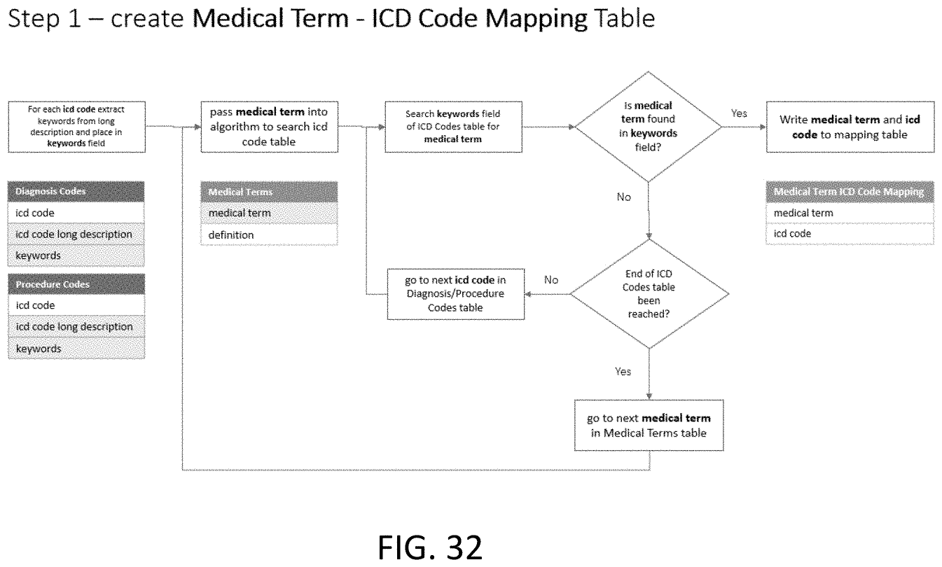

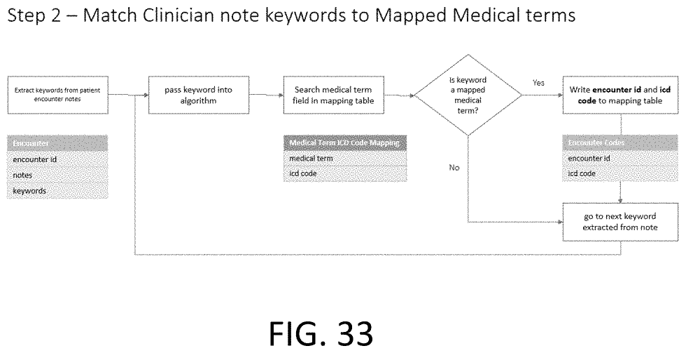

Step 1—An algorithm will be run to extract key words from the long description of each ICD code. These extracted keywords will be stored in a separate column in the table used to store the ICD codes. See FIG. 32.

Step 2—An algorithm is run that will traverse through each record in the ICD code table. For each ICD code table record the algorithm will search the medical term table for any term that is found to be a keyword in the extracted keywords for that code. Thus a database of words/terms comprising keywords of the protocol that also comprise medical terms will be created. See FIG. 33.

Step 3—When a match is found the 1) ICD Code, 2) ICD Code Long Description, and 3) medical term found/matched are written to separate table—an ICD Code-Medical Term Mapping table—that now maps the medical term to the ICD code within which it was found in the code's long description. This “mapping” table is now the key system component that ties medical terms to ICD codes.

NOTE: a single record is created for each medical term found as a keyword for an ICD code. So, if a given ICD code has a long description that contains three (3) keywords that happen to be medical terms then three (3) separate records will be written to the mapping table.

### Example

| ICD Code | Long Description | Medical Term |
|---|---|---|
| 72619 | Other ganglion and cyst of synovium, | bursa |
| | tendon, and bursa | |
| 76249 | Other specified disorders of bursae and | bursae |
| | tendons in shoulder region | |
| 72789 | Other disorders of synovium, tendon, and | bursa |
| | bursa | |
| 72610 | Disorders of bursae and tendons in | bursae |
| | shoulder region, unspecified | |

It is important to note this process is part of the disclosure and creation of software that will be utilized in clinician use during a patient encounter. The mapping table must be prepared prior to use of the solution. Also, it is lengthy as the algorithm must traverse though thousands of medical terms for each of several thousand ICD codes.

Step 4—During a patient encounter a clinician (or similar) enters notes for the encounter. These notes describe the details of what the patient is visiting the physician for—whether diagnosis or procedure.

Step 5—The disclosure uses an artificial intelligence (AI) text analysis, e.g., Keyword Extraction, to extract key words from the clinician notes. Each key word is passed into an algorithm that searches the Medical Term Table to determine if it matches a medical term. This step can include natural language analysis to identify terms as part of machine learning algorithms.

Step 6—When a match is found, the disclosure teaches searching the ICD Code-Medical Term Mapping table to find the medical term and the correlated ICD code.

Step 7—Each ICD Code match from Step 6 is listed as an ICD Code the clinician may select to add to the medical record for the encounter.

Step 8—The clinician may select each desired ICD Code to apply to the encounter (and therefore the EMR). Once each ICD Code is selected, it is added to a table that stores one record for each ICD Code selected. Each record stores an identifier for the encounter and an identifier for the ICD Code. This table is the Encounter/ICD Code table.

Step 9—The system writes and stores a record for each matched key word and the ICD Code it matched to a fact table. This table stores an identifier for the Encounter, the ICD Code, the key word, and a Count (set to a value of 1).

### Invoice Match

Step 1—The ICD Codes provided from the system (as results from Step 7 in the MEDICAL TERM match above) are passed into an algorithm that searches patient invoices.

Step 2—When a match is found, the system writes a record for each matching ICD Code. Each record contains a unique identifier for the invoice item and the associated ICD Code.

Each match found increases the rank of the ICD Code, and therefore the likelihood that code is the correct match for the diagnosis or procedure.

### EMR Match

### Data Preparation.

The system must first be used to import external electronic medical records. Once this data is imported the EMR data is stored in the system and marked as an external record.

Step 1—The ICD Codes provided from the system (as results from Step 7 in the MEDICAL TERM match above) are passed into an algorithm that searches imported medical records.

Step 2—When a match is found the system writes a record for each ICD Code that matches. Each record contains a unique identifier for the external EMR and the associated ICD Code.

Each match found increases the rank of the ICD Code, and therefore the likelihood that code is the correct match for the diagnosis or procedure.

FIG. 9 provides a partial overview of some embodiments of the device and method. The user 1010 can input data and receive search information via a user interface 1011. This interface may include the programed CPU and the display screen discussed above. User may enter text notations and optional search requests 991. In the disclosed embodiment, the system utilizes a Device Manager 910 that may provide access to the patient EHR residing in a File Storage/Document Collection 920. The search results, initiated via 991, may be received via the display 990 of “Review of Search Text & Suggested or Learned Codes”. Also note that the user coding decision 955, based upon suggested codes responsive to the user's text 991, are inputted to the Document Manager 910 and EHR repository 920. The search request data 930 is also accessible to the user via the Document Manager 910

### Expanding Search for Treatment & Test Procedures

The disclosure includes displaying the treatment or test information correlated with suggested ICD-09, ICD-10, CPT or HCPCS diagnosis codes. The information may, in one embodiment, be displayed on hand held devices such as programed iPads, tablet computers, smart phones, lap top computers, etc. utilized by the clinician at the time of patient examination or encounter.

This disclosure also includes the software selecting suggested treatment or test codes relevant to the suggested diagnosis code. In one embodiment, these treatment/test codes may be displayed to the clinician. This information may provide a further opportunity for the clinician to confirm that his/her text description is matching the appropriate code protocol. This will facilitate, among other functions, determining the work is properly coded and appropriate reimbursement paid.

In another embodiment, the test/treatment codes need not be displayed. See FIG. 11 wherein the patient identifying information is displayed 1110 and the clinician identity is also listed 1120. The diagnosis notes are shown 11502a with the suggested codes displayed 11501a. The sections of the display screen wherein the treatment or test codes could be displayed 11501b, 11502b are empty.

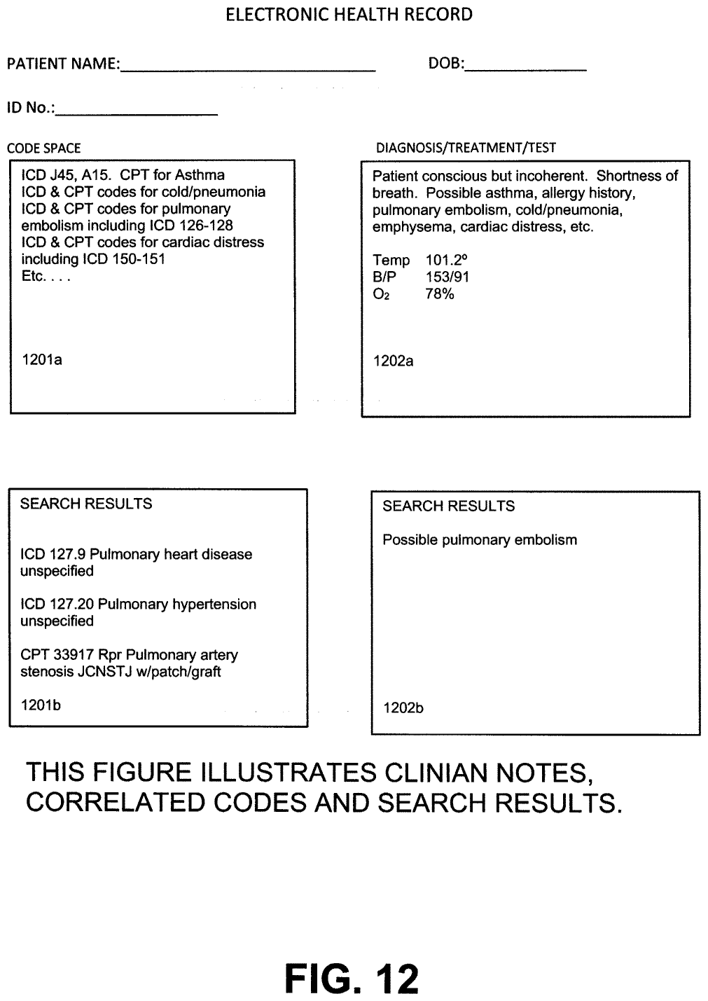

However, in this embodiment, the test/treatment codes will be selected for each software suggested diagnosis code. See FIG. 12 wherein diagnostic information is displayed 1202a and both diagnostic 1201a and treatment codes 1201b are displayed. These test/treatment codes will be included in any search initiated by the clinician. Again, this search will be of the patient's past EHR history. The inclusion of the test/treatment codes in effect broadens the scope of the search to ensure not only records of past diagnoses but relevant tests and treatments that were performed in response to such past diagnoses are presented to the requesting clinician. This embodiment provides an enhanced search.

For example, a patient may be seen with an injured knee. The clinician may note “torn knee ligament”. This may prompt one or several diagnosis codes such as ICD-10 code “S83.209 Unspecified tear of unspecified meniscus, current injury, unspecified knee.” Such code may appear in the code display portion of the clinician's device display screen.

In addition, CPT treatment codes may be prompted. These codes may include but are not limited to:

27405 RPR Primary torn LIGM&/Capsule knee collateral

27407 Repair primary torn LIGM&/Capsule knee cruciate

27409 RPR 1 Torn LIGM&/CAPSL KNE COLTRL&CRUCIATE

29888 ARTHRS AIDED ANT CRUCIATE LIGM RPR/GMNT/RCNSTJ

29889 ARTHRS AIDED PST CRUCIATE LIGM RPR/AGMNT/RCNST

27427 Ligamentous reconstruction knee extra-articular

27428 Ligamentous reconstruction knee intra-articular

27429 Ligamentous RCONSTJ AGMNTJ KNE INTRA-ARTICULAR XTR

27557 Open TX Knee dislocation w/ligamentous repair

27558 Open TX Knee dislocation w/repair/reconstruction

27380 Suture infrapatellar tendon primary

27381 Suture infrapatellar TDN 2 RCNSTJ w/FSCAL/TDN GRF

It will be appreciated that any clinician initiated search based upon the above ICD-10 code will include a search for the above listed CPT codes for treatment. It should also be appreciated that the clinician may rapidly scroll through the suggested codes using components such as shown in FIG. 11. Also the multiple codes may be ranked.

It will be further appreciated that the entry for “27409 RPR 1 Torn LIGM&/CAPSL KNE COLTRL&CRUCIATE” quoted above may be an abbreviated version of the following full CPT entry stated as follows:
“Surgery
Surgical Procedures on the Musculoskeletal System
Surgical Procedures on the Femur (Thigh Region) and Knee Joint
Repair, Revision, and/or Reconstruction Procedures on the Femur (Thigh Region) and Knee Joint
Repair, primary, torn ligament and/or capsule, knee
27409 collateral and cruciate ligaments”

The disclosure includes various forms of abbreviation to facilitate display on a display screen and facilitate ease of use and rapid review of code descriptors.

As discussed above, the contents of the suggested codes may, upon clinician's review, may cause the clinician to revise the text of the symptom or diagnosis description. For example, a clinician's entry of “knee strain” may result in the following ICD diagnosis code being displayed:
“S76.11 Strain of quadriceps muscle, fascia and tendon”

This revision may cause other codes to be suggested. Entry of “cruciate knee ligament strain may return the diagnostic entries including:
“S83.5 Sprain of cruciate ligament of knee
S83.50 Sprain of unspecified cruciate ligament of knee
S83.51 Sprain of anterior cruciate ligament of knee
S83.501 Sprain of unspecified cruciate ligament of right knee
S83.511 Sprain of anterior cruciate ligament of right knee”

In an embodiment of the disclosure, the clinician may be prompted to specify greater detail to the description. Such detail may be whether the injury is to the left or right knee. Further detail may specify whether the strain is to a cruciate or collateral ligament. Further detail may be whether the injury is to the medial collateral ligament, lateral collateral ligament, or super tibiofibul joint and ligament, etc. This indication of further detail may facilitate a more thorough examination.

The clinician may find these alternate codes to be more relevant to the patient symptoms or diagnosis. The software may utilize machine learning to offer or suggest different codes based upon the text of the past entered symptoms/diagnoses and ultimately clinician selection of codes.

It will also be appreciated that the software may identify test procedures which can be performed for the knee injury. The relevant codes may be selected. Such codes may include but are not limited to the following CPT codes:
73560 Radiologic examination knee; 1 or 2 views
73562 Radiologic examination knee; 3 views
73564 Radiologic examination knee; complete, 4 or more views
73565 Radiologic examination, knee; both knees, standing, anteroposterior
73721 MRI any JT Lower EXTREM W/O Contrast MATRL
73722 MRI any JT lower EXTREM W/Contrast MATRL
73723 MRI and JT Lower EXTREM W/O & W/contrast MATRL
01320 ANES Nerve MUSC Tendon Facia & Bursa Knees&/POPLT
1380 thesia Closed Procedures Knee Joint

It will be appreciated that inclusion of the optional test and treatment codes into the electronic search criteria will cause the search to be more robust. Stated differently, the net cast by the clinician initiated search will be improved and with a small mesh. The search will not be limited to the diagnosis selected by the clinician but may include past test and treatment procedures related to the injured or complained area of the patient.

Referring to FIG. 12, the EHR search may disclose a past medical history that provides reasons or causes that may explain why the patient is currently suffering from a shortness of breath. The software, perhaps using machine learning, is programed to identify causes of shortness of breath (as well as other physical conditions or symptoms) and conduct a search of such relevant diagnostic and test and procedure codes.

As the clinician enters observed symptoms or diagnosis information 1202a, the software correlates and suggests code protocols 1201a relevant to the diagnosis or symptoms. These codes may be displayed to the clinician. See FIG. 12. The codes will be understood to include the text descriptors that accompany each alphanumeric code of the code protocols. Where the symptoms do not suggest a single cause, the clinician can utilize a search function of the device. The software, identifying multiple possible causes and correlating these causes to protocol codes, can use these codes as a search index or tool to rapidly and simply electronically review the patient past history via the EMR or EHR. The search disclosure of any one of the CPT and/or ICD codes may identify a likely cause of the symptoms in spite of lack of information received from the patient. For example, the search history may include codes associated with relevant past tests or treatments. Relevant past tests or treatments pertain to tests or treatments that may have previously ordered for the patient for medical disorders associated with the observed symptom, e.g., shortness of breath. If similar codes are found, it can indicate a history of past causation. This can simplify the clinician's initial treatment and testing efforts.

For example, an observation of patient shortness of breath can have multiple causes. As previously suggested, causes may vary from cold, pneumonia, COPD (including emphysema or chronic bronchitis) anxiety, fear, to asthma, CO poisoning, pulmonary embolism, cardiac failure. Diagnostic, test and treatment codes related to these causes may be identified by the software and searched. Utilization of the code protocols provides an effective indexing tool in contrast to utilization of “key words” where such words or terms may vary with clinician or health care entity. It will be appreciated that an immediate electronic search of the patient's EMR or EHR returning in real-time a history (past diagnosis, testing or treatment) for COPD may allow better (beneficial and cost effective) treatment of the patient. This result will be enhanced by such search returning no history pertaining to pulmonary embolism or cardiac failure.

In an embodiment, the software can identify codes for one or more of these causes. For example, the ICD-10 code for unspecified asthma is J45. There are multiple subtopics, e.g.,
J45.2 Mild intermittent asthma
J45.3 Mild persistent asthma
J45.4 Moderate persistent asthma
J45.5 Sever persistent asthma
J45.901 Unspecified with acute exacerbation
J45.902 Unspecified asthma with status asthmaticus
J45.909 Unspecified asthma, uncomplicated
J45.998 Other asthma
J45.90 Unspecified asthma
J45.99 Other asthma

It is suggested that a search for J45 and all subheading would be a productive software search for asthma as a causative factor. As shown below, additional codes related to asthma may be relevant to the symptom of shortness of breath. Utilization of coding indexes and hierarchy of coding sequences may be used. The software can be programed to search all subgroups of a relevant hierarchy. For example, a search would include all subgroups under J45 or A15.

Other ICD codes determined by the software, enhanced by machine learning, may include:
A15.0 Tuberculosis of lung
B44.0 Invasive pulmonary aspergillosis
J60 Coalworker's pneumoconiosis
J62.8 Pneumoconiosis due to other dust containing silica
J64 Unspecified pneumoconiosis

Similarly, the CPT codes could be parsed and codes selected for asthma testing and treatment. For example, the software could identify the following exemplary codes, e.g.,
1005F Asthma symptoms evaluated
2015F Asthma impairment assessed
2016F Asthma risk assessed
5250F Asthma discharge plan present
4015F Persistent asthma long term control medication prescribed
1038F Persistent asthma mild moderate or severe asthma
1039F Intermittent Asthma
94375 Respiratory flow volume loop
94640 Pressurized/non-pressurized inhalation treatment
0276T Bronchoscopy, rigid or flexible, including fluoroscopic guidance, when performed; with bronchial thermoplasty, 1 lobe
94728 Airway resistance by impulse oscillometry

Similarly for cold or pneumonia as the possible cause of observed shortness of breath, the software could identify ICD diagnostic codes including but not limited to:
J13 Pneumonia due to Streptococcus pneumoniae
J14 Pneumonia due to Hemophilus influenzae
J17 Pneumonia in disease classified elsewhere
J16 Pneumonia due to other infectious organisms, not elsewhere classified
J18 Pneumonia, unspecified organism
P23 Congenital pneumonia
A01.03 Typhoid pneumonia
A02.22 Salmonella pneumonia
B01.2 Varicella pneumonia
B06.81 Rubella pneumonia

It will be appreciated that the above is only a partial list of ICD and/or CPT codes responsive to the diagnosis of pneumonia. The software would conduct a comprehensive search of such terms. Again, this search could be conducted immediately and search results displayed to the clinician in real time to aid in the diagnosis and treatment process. It will be appreciated that the search results may therefore show a patient history of treatment for a malady that displays the symptoms being currently observed by the clinician. Depending upon the search results, the clinician may initiate one or more follow-up searches to facilitate assessment of the patient history. The clinician, utilizing the search results, may order tests relative to the past malady. If the test results return negative, the clinician may broaden the scope of the tests.

In additional various CPT as well as other code protocols could be searched for treatments or tests relevant to a diagnosis of pneumonia. Such codes could include but not be limited to:
4279F Pneumocystis jiroveci pneumonia prophylaxis prescribed (HIV)
76604 Ultrasound, chest (includes mediastinum), real time with image documentation
86156 Cold agglutinin screen
86157 Cold agglutinin titer
86329 Immunodiffusion not elsewhere specified
86603 Antibody adenovirus
86631 Antibody chlamydia
86632 Antibody chlamydia IGM
86641 Antibody crytococcus
86668 Antibody Francisella tularensis
86684 Antibody haemophilius influenza
86710 Antibody influenza virsus
86713 Antibody legionella
86738 Antibody mycoplsm
86744 Antibody nocardia

In regard to possible pulmonary embolism, responsive ICD codes could include but are not limited to the following:
I26 Pulmonary embolism
I26.0 Pulmonary embolism with acute cor pulmonale
I26.01 Septic pulmonary embolism with acute cor pulmonale
I26.02 Saddle embolus of pulmonary artery with acute core pulmonale
I26.09 Other pulmonary embolism with acute cor pulmonale
I26-128 Pulmonary heart disease and diseases of pulmonary circulation
127.9 Pulmonary heart disease unspecified
127.20 Pulmonary hypertension unspecified
127.82 Chronic pulmonary embolism
174.10 Embolism and thrombosis of unspecified parts of aorta
174.11 Embolism and thrombosis of other parts of aorta
286.711 history of pulmonary embolism

CPT codes pertaining to pulmonary embolism include but are not limited to:
33910 Pulmonary artery embolectomy with cardiac bypass
75741 Angiography pulmonary unilateral diagnostic radiology procedures of aorta and arteries
75743 Angiography pulmonary bilateral SLCTV RS&I
75746 Angiography pulmonary non-selective CATH/VEN NJX RS&I
33471 Valvotomy pulmonary valve CLSD Heart via pulmonary artery
33475 Replacement pulmonary valve
33917 Repair pulmonary artery stenosis JCNSTJ w/patch/graft
33920 Repair pulmonary atresia w/CONSTJ/RPLCMT conduit
33922 Transection pulmonary artery w/Cardiac bypass

As noted above, one additional cause of shortness of breath can be cardiac failure or similar. The software may display diagnostic codes related to cardiac distress such but not limited to:
I42.0 Dilated cardiomyopathy
I50.9 Heart failure unspecified
I50.1 Left ventricular failure unspecified
I50.20 Unspecified systolic (congestive) heart failure
I50.21 Acute systolic (congestive) heart failure
I50.22 Chronic systolic (congestive) heart failure
I50.30 Unspecified diastolic (congestive) heart failure
I50.31 Acute diastolic (congestive) heart failure
I50.32 Chronic diastolic (congestive) heart failure
I50.89 Other heart failure
I50.82 Biventricular heart failure
I50.810 Right heart failure unspecified
I50.814 Right heart failure due to left heart failure
I51.5 Myocardial degeneration
I51.9 Heart disease unspecified
R57.9 Shock, unspecified

It will be appreciated that this is only a partial list of diagnostic codes that may be prompted by a clinician's notation of the symptom of shortness of breath related to a possible heart condition. As already shown above, treatment or test codes (e.g., CPT codes) will also be generated for the rapid, real-time electronic search of the patient's EHR that may be initiated by the clinician. In an embodiment, the scope and order of test and treatment codes may be a product of machine learning evaluation of the noted symptoms.

Further, and in addition to FIG. 12 mentioned above, FIG. 13 illustrates an embodiment of a display screen 1310 allowing the clinician to swipe through the multiple suggested codes and reject/select codes. It will also be appreciated that FIG. 13 illustrates a display screen format useful for the review of search results. The clinician may scroll 1340 through the documents displayed 1390. The status bar could designate where on the totality of documents the particular document is placed, i.e., number 15 of 35 total retrieved documents. The relevancy 1320 of the documents can be noted by the clinician.

### Speech Recognition and Free Form Text

It will be appreciated that in another embodiment, text descriptions of diagnosis/treatment/tests may be entered by speech recognition technology. This allows the diagnosis/treatment/test entries may be entered by the clinician's spoken words. This allows the clinician to dictate his/her comments without turn away from the patient (as is required if the clinician turns to type into a keyboard). One embodiment of speech recognition technology is described in U.S. Pat. No. 8,670,979 issued to Thomas Robert Gruber et al. on Mar. 11, 2014 and which is incorporated by reference herein in its entirety. Other methods are included in the scope of this application.

The device may also include a function to transcribe hand written text entered on the display screen with digital text.

### Machine Learning

FIG. 14a represents a conceptual overview of a classification system 1400. The classification system utilizes machine learning and conducts key word searches. The EHR Data Entry and Record Search Request 1131 (See FIG. 11) may be any health care or treatment information identifiable to a single patient that can be stored in any electronic format including a word-processing file, spreadsheet, email, text message, contact list, calendar entry, HTML file, image file, video file, source code, object code, post-script file or a digital version of a physical document. An EHR data entry form (See FIG. 11) may be used to electronically create EHR data information pertaining to an identifiable patient 1110 and an identifiable health event 11502a, e.g., an ER visit or annual physical, etc. EHR data may also be handwritten text information, videos, images, etc. that are recorded and transportable/displayable in electronic format. The document collection can comprise multiple sources of EHR data stored in separate locations. It may also comprise a database. The document collection may also refer to an EHR data entry form.

Continuing with FIG. 14a, the classification system 1400 is utilized by each code protocol to organize the different diagnosis/treatment/text procedures. For example, diagnoses pertaining to the knee will be in one class with numerous subclasses (sub categories). Each class 1430 and subclass 1440 will be assigned a different alphanumeric code. The structure of the code may disclose its class and subclass, etc. It will be appreciated that each subclass may also have additional subclasses, thus creating a multi-level hierarchy of classes and subclasses. The hierarchy may be disclosed by the alphanumeric code.

Each class, subclass, and sub-subclass will comprise a distinct code and code descriptor.

A medical issue described in the EHR data entry form 11502a of FIG. 11 may be classified as a member of any number of classes 1430 or subclasses 1440 defined for a classification problem. For example, class 1 might represent the set of medical issues related to a patient's knee(s), while class 2 might represent a set of medical issues related to the pelvis, femur and/or hip joint, all as described in the health care provider's text data entry. A subclass 1 of class 1 might further classify medical issues as potentially relevant to the meniscus, while subclass 2 of class 1 might refer to medical issues related to the ligaments of the knee. There may yet be a third subclass (not shown) related to issues of the knee cap (patella). A further sub-subclass distinguishing between the left knee and the right knee. A yet further sub-subclass may pertain to whether this is the first or a subsequent issue pertaining to the knee.

It will be appreciated that in some instances, EHR data not previously indexed by the use of a coding protocol as taught by this disclosure, may be indexed utilizing the classification system 1400 as shown in FIG. 14a in response to a search request of a clinician. The classification system may contain one or more optical character readers (OCR). After such classification step is performed, the now indexed data may be parsed for codes and text responsive to the search request.

The EHR data may be indexed utilizing keyword techniques to match the text with the code descriptors and code identifiers. This may require segregation of terms as nouns comprising locations such arm, elbow, wrist, femur, tibia, pelvis noted in the text of the EHR record with codes containing these locations words in the code descriptor. In some instances, heath care records that have been previously coded for billing purposes may be utilized as a resource for EHR code classification.

Data Entry & Code Protocol Integration into EHR

The Applicant's disclosure allows the machine learning to change/amend/supplement the descriptors to encompass the clinician's entry. The process, under taken by machine or active learning facilitates the translation of the clinician's text into the universally understood common language. The amended identifier will reduce the number of “partial matches”, thus making the clinician's job of entering appropriate codes substantially easier.

Further, this disclosure includes embodiments wherein the code selected by the clinician is imbedded into the EHR. The disclosure includes machine learning and resulting modification of the code descriptors (becoming code identifiers) to expand the identifier to more closely match or encompass the text used by the clinician.

Further, the active machine learning subject of this disclosure attempts to understand the clinician's text entry and correlate it to one or more alternate code protocol descriptors. The object is to get the clinician's feedback in real time and the modify or amend the descriptors so that the text entries are correlated to the code descriptors and vice versa. This modification is achieved through machine learning.

The disclosure also learns from the selection of the code, presumably based on the code descriptor (where the code descriptor is part of the original code protocol) or later created code identifier. The clinician creates an EHR entry using his/her own text language to describe the condition, etc. The machine learning system evaluates the text.

For example, the clinician may describe a patient condition in multiple different ways:
a. “Complaint of knee pain.”
b. “Patient complains of pain in the knee.”
c. “Patient can't run due to pain in the knee”
d. “Patient can't run due to pain in his knee.”
e. “The patient states his knee is sore after running and it hurts to stand from a sitting position.”
f. “Complaint of pain in his knee when he rotates his foot (and ankle).”
g. “Patient complains that his knee hurts when he bends his leg (at the knee).”
h. “Patient's knee hurts when he stands an places weight on his knee.”
i. “Patient's knee hurts when he climbs stairs.”
j. “Patient knee is swollen on one side and shows edema”
k. “Patient's knee is sore and red. Also tender on left side.”

At initiation of the system, the disclosure may evaluate the text entry to identify the subject matter, i.e., knee and the adjective used to further describe the knee. The disclosure may perform a key word search based upon:
i. Location of the event/complaint/symptom etc., Here “knee” and (initially at startup) suggests codes and code descriptors.
ii. Symptom complained of, here “pain”, “sore” “hurt” & “tender”
iii. Symptoms observed: redness, swelling, edema
iv. Other factors can be searched such as “is this the first time of complaint” See ICD descriptors.
v. Duration (see above)

The system may perform multiple keyword searches (502, 503, 504 of FIG. 5) of multiple code protocols (or at least one code protocol selected by the health care provider or health care institution). This search may produce multiple suggested codes.

In one embodiment, if the clinician simply enters “knee”, the system may respond with a display of “Too broad” in the window or pane used to display suggested codes. This may prompt the health care provider to further define the symptoms.

In one embodiment, the search may be “knee pain”, “pain in knee”,
i. “swollen knee” “knee swollen”, “knee swelling”
ii. “hurt knee”, “knee hurt”
iii. “knee edema”

The search results for ICD-10 code protocol for each search are:
i. “knee”, “knee pain”, “pain in knee”,
   “Complete matches”
1. M25.56 Pain in knee
2. M25.561 Pain in right knee
3. M25.562 Pain in left knee
4. M25.569 Pain in unspecified knee
5. T84.84 Pain due to internal orthopedic prosthetic devices, implants and grafts
6. M22.2X9 Patellofemoral disorders, unspecified knee
   “Partial matches” Selected (approximately 150 responses displayed)
7. T85.840 Pain due to nervous system prosthetic devices, implants and grafts
8. T85.848 Pain due to other internal prosthetic devices, implants and grafts
9. A18.02 Tuberculous arthritis of other joints
10. M17 Osteoarthritis of knee
11. M24.56 Contracture, knee
12. M24.66 Ankylosis, knee
13. M25.16 Fistula, knee
14. M25.46 Effusion, knee
15. M25.76 Osteophyte, knee
16. M67.46 Ganglion, knee
17. M94.26 Chondromalacia, knee

Search “swollen knee” “knee swollen”, “knee swelling”
“Complete matches”
1. M25.469 Effusion, unspecified knee
“Partial matches”
1. M17 Osteoarthritis of knee
2. M24.56 Contracture, knee
3. M24.561 Contracture, right knee
4. M24.562 Contracture, left knee
5. M24.569 Contracture, unspecified knee
6. M24.66 Ankylosis, knee
   a. M24.661 Ankylosis, right knee
   b. M24.662 Ankylosis, left knee
   c. M24.669 Ankylosis, unspecified knee
7. M25.06 Hemarthrosis, knee
   a. M25.061 Hemarthrosis, right knee
   b. M25.062 Hemarthrosis, left knee
   c. M25.069 Hemarthrosis, unspecified knee
8. M25.16 Fistula, knee
   a. M25.161 Fistula right knee
   b. M25.162 Fistula left knee
   c. M25.169 Fistula unspecified knee
9. M25.46 Effusion, knee
   a. M25.461 Effusion right knee
   b. M25.462 Effusion left knee
10. M25.76 Osteophyte, knee
   a. M25.761 Osteophyte, right knee
   b. M25.762 Osteophyte, left knee
   c. M25.769 Osteophyte, unspecified knee
11. M67.46 Ganglion, knee
   a. M67.461 Ganglion, right knee
   b. M67.462 Ganglion, left knee
   c. M67.469 Ganglion, unspecified knee
12. M94.26 Chondromalacia, knee
   a. M94.261 Chondromalacia, right knee
   b. M94.262 Chondromalacia, left knee
   c. M94.269 Chondromalacia, unspecified knee
13. M17.9 Osteoarthritis of knee, unspecified
14. S80.21 Abrasion of knee
   a. S80.211A Abrasion, right knee, initial encounter
   b. S80.211D Abrasion, right knee, subsequent encounter
   c. S80.211S Abrasion, right knee, sequela
   d. S80.212A Abrasion, left knee, initial encounter
   e. S80.212D Abrasion, left knee, subsequent encounter
   f. S80.212S Abrasion, left knee, sequela
   g. S80.219A Abrasion, unspecified knee, initial encounter
   h. S80.219D Abrasion, unspecified knee, subsequent encounter
   i. S80.219S Abrasion, unspecified knee, sequela
15. S80-S589 Injuries to the knee and lower leg
16.S80 Superficial injury of knee and lower leg
17. S80.0 Contusion of knee
18. S81 Open wound of knee and lower leg
19. M00.06 Staphylococcal arthritis, knee
20. M00.16 Pneumococcal arthritis, knee
21. M02.16 Postdysenteric arthropathy, knee
22. M02.26 Postimmunization arthropathy, knee
23. M02.36 Reiter's disease, knee
24. M05.06 Felty's syndrome, knee
25. M06.26 Rheumatoid bursitis, knee
26. M06.36 Rheumatoid nodule, knee
27. M07.66 Enteropathic arthropathies, knee
28. M10.06 Idiopathic gout knee
29. M10.16 Lead-induced gout, knee
30. M10.26 Drug-induced gout, knee
31. M11.16 Familial chondrocalcinosis, knee
32. M11.26 Other chondrocalcinosis, knee
33. M12.16 Kaschin-Beck disease, knee
34. M12.36 Palindromic rheumatism, knee
   a. M12.361 Palindromic rheumatism right knee
   b. M12.362 Palindromic rheumatism left knee
   c. M12.369 Palindromic rheumatism unspecified knee
35. M12.46 Intermittent hydrarthrosis, knee
   a. M12.461 Intermittent hydarthrosis, right knee
   b. M12.462 Intermittent hydarthrosis, left knee
36. M12.56 Traumatic arthopathy, knee
   a. M12.562 Traumatic arthopathy left knee
   b. M12.569
37. M14.66 Charcot's joint, knee
   a. M14.661 Charcot's joint, right knee
   b. M14.662
   c. M14.669
38. M21.26 Flexion deformity, knee
   a. M21.261
   b. M21.262
   c. M21.269
39. M23 Internal derangement of knee
40. M24.46 Recurrent dislocation, knee
   a. M24.461 Recurrent dislocation, right knee
   b. M24.462.Recurrent dislocation, left knee
41. M25.26 Flail joint, knee
   a. M25.261 Flail joint, right knee
   b. M25.261 Flail joint, left knee
   c. M25.269. Flail joint unspecified knee
42. M25.36 Other instability, knee
   a. M25.369 Other instability, unspecified knee
43. M25.56 Pain in knee
   a. M25.561 Pain in right knee
   b. M25.562 Pain in left knee
44. M25.66 Stiffness of knee, not elsewhere classified
   a. M25.662 Stiffness of left knee
   b. M25.669 Stiffness of knee, not elsewhere classified
45. M67.36 Transient synovitis, knee
   a. M67,361 Transient synovitis, right knee
   b. M67.362 Transient synovitis, left knee
   c. M67.369 Transient synovitis, unspecified knee
46. M93.26 Osteochondritis dissecans knee
   a. M22.2X1 Patellofemoral disorders, right knee
   b. M22.2X2 Patellofemoral disorders, left knee
   c. M22.2X9 Patellofemoral disorders, unspecified knee
47. M22.40 Chondromalacia patellae, unspecified knee
   a. M22.41 Chondromalacia patellae right knee
   b. M22.42 Chondromalacia patellae left knee
48. M23.40 Loose body in knee, unspecified knee
   a. M23.41 Loose body in knee, right knee
   b. M23.42 Loose body in knee, left knee
49. M23.52 Chronic instability of knee, left knee
50. M67.50 Plica syndrome, unspecified knee

In view of the size of responsive entries for search terms “swollen knee” “knee swollen”, “knee swelling”, the coding system may display “Too Broad”. This may prompt further defining text to be entered by the health care provider. Upon the clinician's modification or narrowing of the entry, the search of appropriate codes may be automatically repeated. (See FIG. 6, 612, 612A, 601) It will be appreciated that this activity includes review of literature, etc. 604, in conjunction with machine learning as well as review of a combined data base 608. Note that this activity can occur during the patient encounter. Therefore code entries can be precisely defined. This is in contrast to current practice wherein coding occurs “after the fact” and may be performed by a third party interpreting the health care provider's cryptic notes.

In another example:
i. Search “hurt knee”, “knee hurt”
   1. No complete match, approximately 150 partial matches. List of partial matches is the same as results for “swollen knee”
ii. Again, due to the large number of responsive partial matches, the system may again respond with “Too Broad”.
   Search “knee edema

No complete matches

Partial matches
1. M17 Osteoarthritis of knee
   a. M17.9 Osteoarthritis of knee unspecified
2. M24.56 Contracture, knee
   a. M24.561 Contracture, right knee
   b. M24.562 Contracture, left knee
   c. M24.569 Contracture, unspecified knee
3. M24.66 Ankylosis, knee
   a. M24.661 Ankylosis right knee
   b. M24.662 Ankylosis left knee
   c. M24.669 Ankylosis unspecified knee
4. M25.06 Hemarthrosis, knee
   a. M25.061 Hemarthrosis, right knee
   b. M25.062 Hemarthrosis, left knee
   c. M25.069 Hemarthrosis, unspecified knee
5. M25.16 Fistula, knee
   a. M25.161 Fistula, right knee
   b. M25.162 Fistula, left knee
   c. M25.169 Fistula, unspecified knee
6. M25.46 Effusion, knee
   a. M25.461 Effusion, right knee
   b. M25.462 Effusion, left knee
   c. M25.469 Effusion, unspecified knee
7. M25.76 Osteophyte, knee
   a. M25.761 Osteophyte, right knee
   b. M25.762 Osteophyte, left knee
   c. M25.769 Osteophyte, unspecified knee
8. M67.46 Ganglion, knee
   a. M67.461 Ganglion, right knee
   b. M67.462 Ganglion, left knee
   c. M67.469 Ganglion, unspecified knee
9. M94.26 Chondromalacia, knee
   a. M94.261 Chondromalacia, right knee
   b. M94.262 Chondromalacia, left knee
   c. M94.269 Chondromalacia, unspecified knee

In this instance, the system may only display the general category (class) for M17 “Osteoarthritis of knee”, M24.56 “Contracture, knee”, M24.66 “Ankylosis, knee”, M25.06 “Hemarthrosis, knee”, etc.

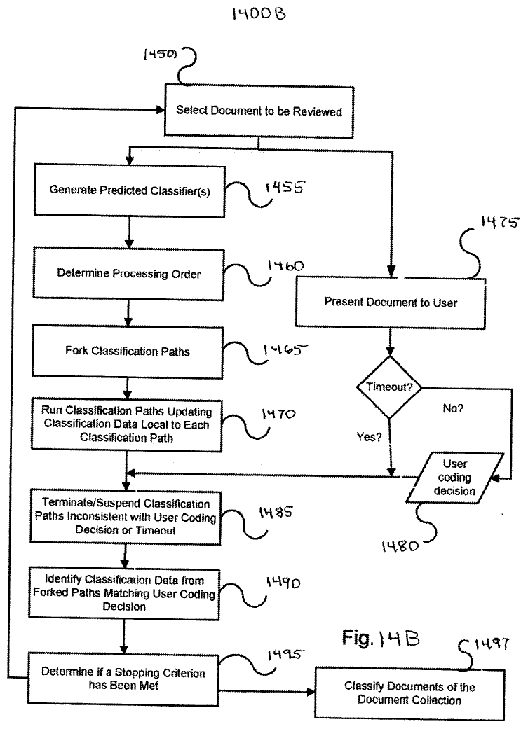

In the embodiment of the interface display 1320 illustrated in FIG. 13, the clinician's EHR entry search results appears in the display window 1390. This may be the clinician device 1100 of FIG. 11 (or 1520 of FIG. 15). In an embodiment of FIG. 13, portions of the existing EHR are displayed. The display window 1390 may include a scroll bar 1370 to facilitate review of the current document should it be too large for display window.

Clinician search result interface 1310 (FIG. 13) may also include a number of user interface review elements for entering user coding decisions 1350. The interface also includes a display for suggested codes and code descriptors 1390. In an embodiment, the clinician may designate one or more codes for display.

Alternatively, the clinician may enter a code for a class designation and the processor will respond with a display of subclasses listings. The clinician may then select among the subclass listings.

Referring to FIG. 15, the clinician can accept one or more of the displayed codes for indexed entry into the EHR (along with the clinician's text diagnosis/treatment/test entry). This acceptance can be via the input device 1528 of the client device 1520, e.g., highlighting and pressing the keyboard enter key, using a mouse or similar device by clicking on a highlighted entry, etc. It will be appreciated that this step can reduce, and perhaps eliminate, the need for review by a later medical coder. The codes entered by the clinician, suggested by the system of this disclosure utilizing machine learning, may be used for both EHR indexing and billing. The system facilitates more prompt payment and prompt searching of past EHR entries for productive administration of health care, e.g., eliminating duplicate tests.

Referring to FIG. 9, some status elements that may be used to indicate the coding decisions 955, may be used in conjunction with other codes (e.g., flag). User interface elements may also include a text or edit box 991 for adding notes to a user coding decision 955 of FIG. 9, which allows for an elaboration on a decision to flag a document. Similar user interface elements (scroll bar 1105 of FIG. 11 or text display arrows 1370 of FIG. 13, etc.) may be presented for each class 1430 and/or subclass 1440 of FIG. 14a. Alternatively, user 1010 may be presented with a user interface review element (e.g., list box 11501a (FIG. 11) and/or arrows 1340 (FIG. 13) for selecting a class 1430 and/or subclass 1440 (FIG. 14a) of the classification problem and selecting, applying and recording user coding decisions 955 of FIG. 9. In some embodiments, instead of indicating a class 1430 or subclass 1440 for a user coding decision 955 by having user 1010 select a class 1430 or subclass 1440 using a suitable interface element, user interface 950 informs the user of the class 1430 or subclass 1440 being reviewed. For example, user interface 1011 may indicate the current class 1430 or subclass 1440 under review to user 1010 using a suitable interface element 1011 (e.g., status bar 1380 of FIG. 13).

2 Referring to FIG. 13, user interface 1320 may also include a number of interface elements 1340 for navigating the clinician's EHR entry or displayed codes and code descriptors. A submit button 1350 or 1132 (See FIG. 11) may also be presented for recording or accepting the clinician's EHR entry or related coding decision. Use of navigation or other user interface elements may trigger error-checking code or subroutines to apprise the user of a possible error condition e.g., no coding decision made (See FIG. 13 1341).

Referring to FIGS. 11 and 13, a user interface for review of an EHR entry 11501a, 11501b, etc. or 1390 may also include an undo operation. An undo operation may be executed by the clinician 1010 (FIG. 9) interfacing with an appropriate interface element for example, undo button 1341 as shown in FIG. 13. An undo operation may also reverse the effects of the immediately preceding user coding decision 1350. An undo operation may also, or alternatively, change the document under review 1390 (e.g., reverting back to a previous EHR entry made by the clinician). An undo operation may be used iteratively to undo the effects of a series of user interface operations.

Additionally referring to FIG. 13, clinician may be presented with interface elements (e.g., a search box 1360) for finding, locating and highlighting text strings within the current document under review 1390. Those documents of an initial document set that are selected using a keyword-type search may also be displayed with those keywords found in document under review 1390 highlighted in the display window. The clinician may also be presented with an interface 1370 for skipping from one found keyword or text-string to another.

Continuing with FIG. 13, clinician interface 1320 may also include a status bar 1380 indicating, for example, patient name, DOB, social security number, assigned patient health care provider patient number, etc. The clinician may change the EHR entry under review 1390.

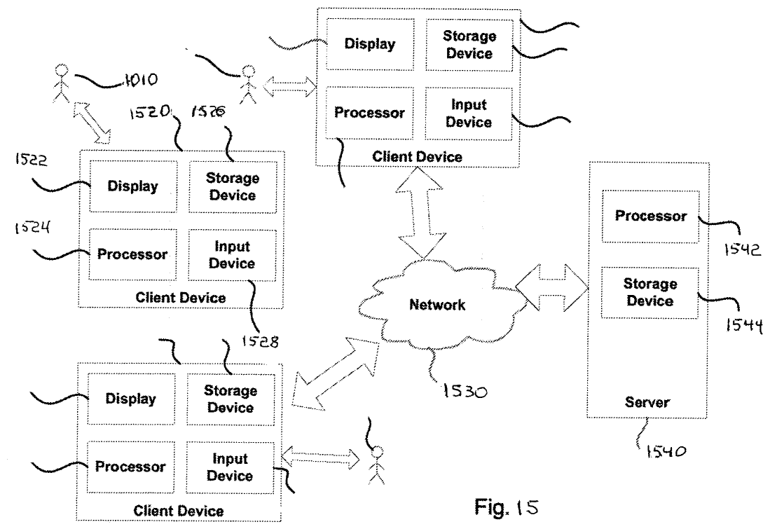

FIG. 9 illustrates how clinician interface 1011 (which corresponds to item 1320 of FIG. 13 and item 1520 of FIG. 15) may communicate and coordinate with document manager 1610 (referring to FIG. 16) to access an existing EHR from a document collection 1420 (referring to FIG. 14a). For example, user interface 1320 (FIG. 13) may issue commands to document manager 1610 in order to retrieve a portion of an indexed EHR for a specific patient using code descriptors and patient identifiers during the health care provider is creating a new EHR entry. As illustrated in FIG. 9, the clinician coding decisions 955 for each EHR entry under review 1490 are captured by the classification system and may be appended to the EHR entry or stored in a separate file or database. Capture of user coding decisions 955 may be implemented at document manager 1610 (FIG. 16).

Referring to FIG. 15, the image of a selected portions of an existing EHR entry or the clinician's current entry and any user interface review elements may be implemented as windows or panes of a single display, separate displays, or a combination thereof of device display 1522. Some or all of the user interface elements may be presented as an overlay on top of the current document to reviewed, such that the user interface review elements will remain stationary while the user scrolls through the document. The clinician 1010 may be given the option of selecting a number of different layouts for user interface display 1520. In one embodiment, the display format 1522 may vary depending upon whether the clinician is entering patient observations and possible diagnosis or is reviewing prior relevant EHR extracts. See FIGS. 10(a) through 10(f), 11 and 13 above. For example, entered data may be displayed in a different font or color or background that the display of past EHR entries.

User interface 1310 (FIG. 13) may be implemented in any suitable programming language (e.g., C, Java) or in a browser (e.g., Internet Explorer, Chrome, Firefox, Safari) using a document markup language (e.g., HTML or XML). User interface 1310 may be a natively programmed application or served to a user device from a remote location.

Alternatively, user interface 1000 (FIGS. 10a-10f) 1100 (FIG. 11) or 1310 (FIG. 13), may be simple and minimalist. In some embodiments, user interface is a terminal (text) interface. A simple implementation of user interface 1100 (FIG. 11) allows classification system 1400 (FIG. 14a) to dedicate further resources to document processing, thus allowing for faster operation of the platform.

User interface 1100 (FIG. 11) may also map the operations of graphical elements to keystrokes on a keyboard. For example, in some embodiments, clinician 1010 (FIG. 15) indicates acceptance of an EHR entry or code by pressing an identified key on a standard keyboard (e.g., “a”) and indicates rejection by pressing a different key (e.g., “g”). By mapping the operations of graphical elements to keyboard keys, a user 1010 can process documents faster by avoiding time consuming user interaction with a GUI pointing device (e.g., a mouse). The clinician may also utilize the keyboard arrow keys to scroll through the displayed document.

Some or all user interface elements may be mapped to a gesture recognition system, whereby the user may navigate an initial document set or make user coding decisions 955 (FIG. 9) by issuing a swipe or sweep gesture on a touch sensitive surface (e.g., touchscreen or touchpad) or in view of a camera, instead of using a button or other user similar interface element. For example, vertical swipes may indicate acceptance or rejection while horizontal sweeps may change the entry or code under review 11501a. Gesture recognition may be performed by the software of user interface 1100 or in a separate software module or library that interoperates with user interface 1100. In certain embodiments, voice recognition is performed where a user performs actions by speaking words or phrases. For example, a user may indicate acceptance by speaking a word or phrase and rejection by speaking a different word or phrase.

In other embodiments, a clinician 1010 (FIGS. 9 & 15) may not need to review any initial document sets. For example, a randomly selected subset of documents from document collection 1420 (FIG. 14a) may be used to approximate a set of non-relevant documents. This randomly selected set bypasses user determination and coding and is instead coded by classification system 1400 of FIG. 14a to be non-relevant (e.g., belonging to class N) and is thus also non-relevant to all subclasses 1440 of the classification problem. Subsequent review and classification by classification system 1400 or a user 1010 may alter this coding decision.

In certain embodiments, e.g., when merging active learning (discussed below) into a single phase, clinician interface 1100 of FIG. 11 need not be provided and instead classification system 1400 of FIG. 14a may rely solely on an active learning interface for document review during active learning iterations.

Machine learning systems are able to update code descriptors or classifiers, and hence the training set, using further human review of selected documents. Documents, i.e., coded EHR entries, may be selected for review based on one or more factors and/or algorithms discussed further below. The selected documents are then reviewed. The review may consist of comparison of the EHR entry and the assigned code/code descriptor or code classifiers. Amended code descriptors (code identifiers) can be reviewed in relation to the EHR entry and the original code descriptors. This review will improve the relevancy of the code descriptors with the EHR entries as created by the clinician. This in turn will improve the accuracy of the code to billing decisions. It will be appreciated that the alphanumeric codes are not amended or modified by the system. Only the code descriptors or identifiers are amended to enhance relevancy to clinician EHR entries and to facilitate the correct code is assigned to the diagnosis/treatment/test. Correct code assignment thereby further improving the effectiveness of the EHR classification/indexing system and the effectiveness of the coding protocol for medical expense reimbursement purposes.

A health care provider/clinician 1010 may request that coding assignment system 1400 (FIG. 14a) use an active learning process during classification. The clinician 1010 may instruct the system to use active learning by interfacing with classification system 1400 using a GUI, a web-interface presented by the classification system or through a command line or any other suitable mechanism. In certain embodiments, classification system 1400 may default to using active learning. The user may be an experienced health care code protocol coder and another experienced clinician.

FIG. 14b illustrates one embodiment for the document classification step utilizing machine learning 1400B. A document responsive to a search code may be selected 1450. A predicted classifier is identified 1455 and a processing order is determined 1460. A forked classification path may be selected 1465 and a running of classification paths may be performed updating classification data to each classification path 1470. The clinician may alternatively be presented with responsive documents 1475 and the clinician may make a coding selection decision 1480. This decision can be integrated with the classification path results 1470. The clinician coding decision may result in exclusion of some classification results 1485 and the documents selected by the forked path process matched with clinician selected codes 1490. Assuming the process is deemed complete 1495, the classified documents can be presented to the clinician 1497.

FIG. 3 illustrates an embodiment of the disclosure wherein the health care provider begins entry of patient observations or preliminary diagnosis 302. This may be at the initial patient encounter. The programed device may begin inserting suggested codes 303. The clinician text may appear in the window 1001a of clinician display 1000 of FIGS. 10a-10f and 11502a, 11502b of the clinician display 1100 of FIG. 11). The system may perform a word search from the entered text and compares the text with the code descriptors of the code protocol 303. The suggested codes may be displayed in window 1001a (FIGS. 10a-10f) or 11501a of FIG. 11 or in 1390 (FIG. 13).

Referring now to FIG. 3, the clinician may accept one or more of the suggested codes 304 or may enter one or more different codes or alter the code descriptors 305. Note the alternate codes or modified code descriptors are transmitted to the code protocol 303 or 307 as part of the machine learning function. The codes (original code suggestions, modified code descriptors or accepted original suggested codes) may be accepted by the clinician 306 and the codes and code descriptors may be integrated into the EHR. Again, the user (clinician) may revise the code descriptor to the code (alphanumeric code identifier) which may be incorporated into the programed CPU/device as part of machine learning. Note the code selection may occur as shown in 1132 of FIG. 11 and 1350 of FIG. 13.

### Machine Learning

Referencing FIG. 18, it will be appreciated that any document selection method may be used at any active learning iteration. For example, active learning module 1160 (FIG. 16) may select an EHR entry based upon the health care provider's text entry or selected code. Alternatively, the clinician/health care provider 1010 (FIG. 15) may select an EHR from document collection 1420 (FIG. 14a) using a user interface 1520 of FIG. 15 or 1812 (FIG. 18) provided by classification system 900 of FIG. 9. For example, user interface 1812 may list relevant text of entered by the clinician. (See 1800 of FIG. 18 generally. The storage device may be equivalent to the storage device 1420 of FIG. 14a, etc.) The text may comprise text extracted from the patient's existing EHR in a window 1001a or pane of a window 11501a in a clinician's device FIG. 10 or 11. The list of EHR entries presented to the clinician from the documents may be ranked or ordered. For example, the clinician 1010 may enter keywords or rules into a device window or text box. Document collection 1420 may be searched with the entered keywords using an algorithm such as BM25 (OKAPI) or any other suitable algorithm in order to provide relevance rankings. The listing of relevant EHR entries presented to clinician 1010 may be ranked accordingly. Alternatively, the document list may be ordered according to scores calculated by classification process 1400 (e.g., using priority queues). The order of the list presented to the clinician 1010 may be updated in real-time.

In another embodiment, the active learning component may select prior selected codes and code descriptors or code identifiers from evaluated EHR's of multiple patients. It will be appreciated that no information concerning individual patients will be disclosed. The machine evaluation will be for searching for alternate classifications of observed conditions or diagnoses. For example, has an entry of “sprained knee” been classified M25.56 Pain in knee or M25.66 Stiffness of knee, not elsewhere classified.

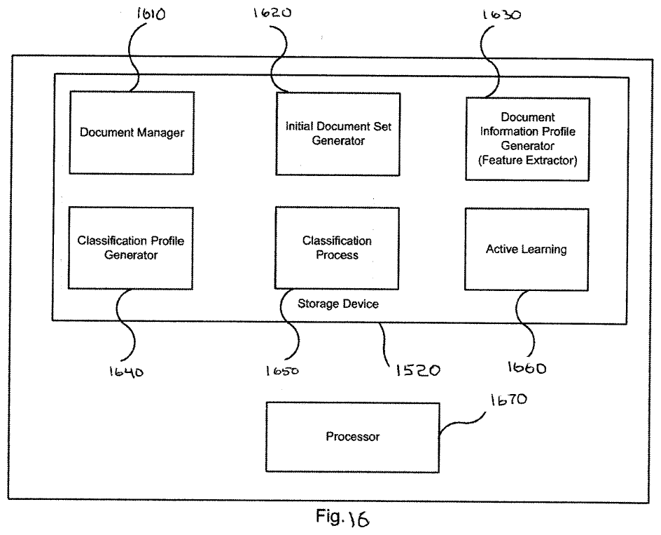

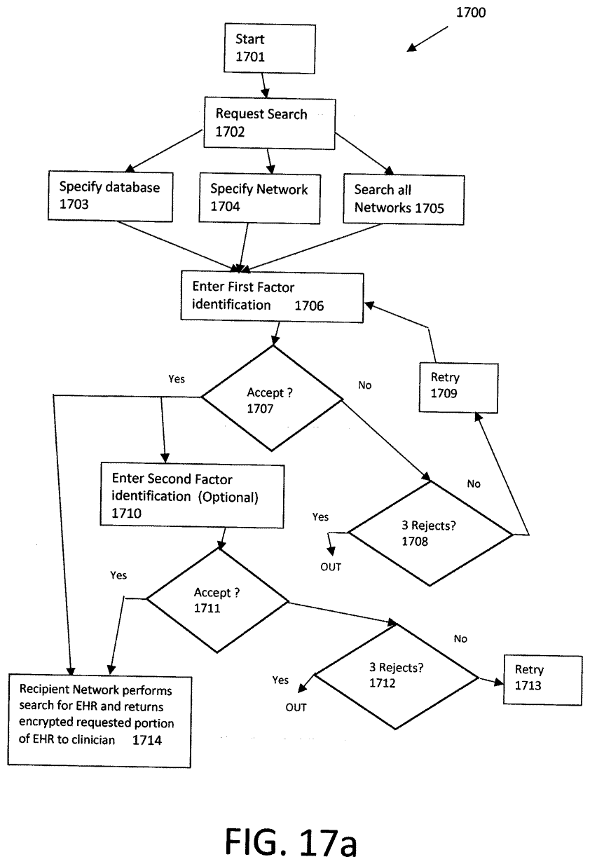

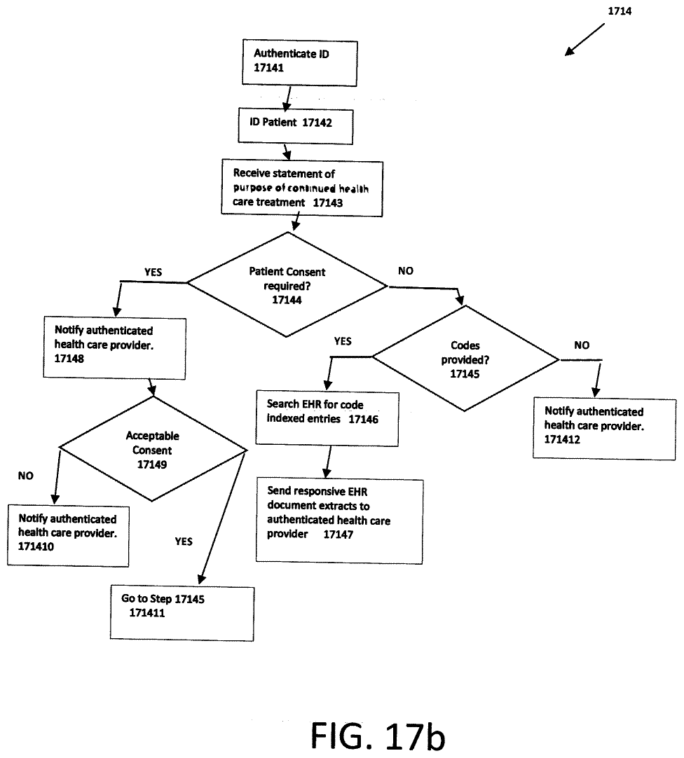

Referencing FIG. 17a, the process 1700 begins with a search request 1702 where the clinician may request a broad or narrow search 1703, 1704, 1705. Reference the clinician's choices 1130 depicted in the screen display 1100 of FIG. 11. Note that the search may require authentication of the clinician in 1704 consistent with the screen display entries 1120 of FIG. 11. In the embodiment illustrated in FIG. 17a, the clinician has three opportunities to successfully input the First Factor authentication 1709. The clinician's credentials may be accepted 1707 wherein a second authentication factor may optionally be required 1710. If the credentials are rejected 3 times 1712, the clinician may not proceed. In other embodiments, further authentication tests may be required or the clinician may be simply denied access to the search function. Assuming the credentialing requirement is satisfied, the search is conducted 1714

Referencing FIG. 17b, an embodiment of the steps of the search function 1714A are provided in greater detail. The clinician authentication has been confirmed 17141 and the patient identity is also confirmed 17142. Recall the patient identity information requirements 1110 of FIG. 11, etc. The search request may contain confirmation that the purpose of the search is for treatment of the identified patient 17143. Recall HIPAA consent is not required for disclosure of patient records used for treatment of the patient. Assuming a search contains suggested codes (see display window 11501a etc. of FIG. 11) provided as part of the search request 17145, the search 17146 is conducted and results transmitted 17147 to the clinician.

In certain embodiments, e.g., when merging active learning into a single phase, document selection step 17146 may be used to select a document that contributes to the overall objectives of classification system 1400, namely, achieving high recall, high precision and using minimal human effort. For example, EHR entry selection step may select an EHR entry based upon its position in a priority queue. For instance, to select the most likely relevant document to a specified code class or subclass, an EHR entry may be selected at step 1714 from the top of a priority queue. High precision and high recall can be realized by minimizing overtraining and identifying additional types of relevant documents. Assuming that EHR entries near the top of a priority queue are similar, it may be beneficial to select an EHR document from a position near the top of a priority queue or elsewhere within the queue, as opposed to purely from the top of the queue, or a random EHR entry to achieve greater sampling diversity.

To determine which document to select from a priority queue, different techniques may be utilized. It will be appreciated that it is the EHR entries that are of interest. The EHR document will contain numerous entries. An EHR document is likely too voluminous to be of meaningful interest due to time required to parse the entire EHR. The entries are selected by one of the alternate methods discussed above. In an embodiment, the entries within the EHR containing codes relevant to the search may be first selected for clinician review.

For example, the process may skip ahead to an EHR entry based on the frequency or number of entries having a similar score that were coded similarly in the same class or subclass. Alternatively, the process may skip EHR entries having relatively similar scores to another set of EHR entries that may be separated by a gap in the queue. Alternatively, an EHR entry may be selected by evaluating the information content of candidate documents. For example, a distance may be calculated between the candidate document and the previously selected document or a series of previously selected documents. More specifically, such a distance may be calculated using the document information profile of the documents and a weighting technique (e.g., nearest neighbor, cosine, Jaccard index). To avoid overtraining, a document may be selected when the distance is inside (similar) or outside a boundary (dissimilar). Alternatively, some other techniques used in rule-based or unsupervised learning techniques may be used (e.g., clustering, threading). For example, documents of the collection may be clustered based upon their similarity and a document may be selected at random from a cluster different from that of the previously selected document or the series of previously selected documents. A document may also be selected by using received user coding decisions. For example, a random document may be selected when a certain percentage of received health care provider coding decisions in a series of health care provider coding decisions have assigned the same code.

In certain embodiments, diversity may be achieved by rotating the EHR entry document selection among a number of different ranking techniques. For example, at one iteration, a document may be selected from the results returned by a keyword search of the document collection or by comparing the documents with an exemplary document (e.g., a document from or outside of the collection) provided by the clinician. At a subsequent iteration, a document may be selected using the results returned by a different keyword search of the document collection, according to a rank in a priority queue, or by a comparison with a different exemplary document provided by the clinician. Move-to-front pooling may be one technique used to rotate document selection among different ranking techniques. More specifically, move-to-front pooling prioritizes the ranking techniques that are used for document selection. For instance, move-to-front pooling may prioritize rankings techniques that are determined by the human reviewer to yield a higher preponderance of relevant documents, for example, through analysis of clinician coding decisions.

Alternatively, a document may be selected at step 1714 (FIG. 17b) according to how closely scores for a document are to one or more calculated threshold values that are used to classify documents from the document collection into classes or subclasses, preferably using a calculated threshold that maximizes a stopping criterion. For example, document selection 1714 may select the document closest to the threshold (i.e., through uncertainty sampling).

After selection by an active learning module or a user, (See FIG. 14b) the document or a reference to the document, may be transmitted to clinician 1010 for review. The clinician may review the EHR text entry and code through an interface (e.g., active learning interface 1660, FIG. 16). In certain embodiments, the interface may be a graphical or textual user interface presented on a display coupled to the classification system. Tightly coupling document selection and presentation (e.g., at the same computer) allows the classification system to minimize latency between active learning iterations.

A user or set of users who may be selected by the system to classify documents retrieved at step 1714 of the active learning process (FIG. 17a) may be specified by a prior clinician 1010, for example, during initialization of the classification problem or at any later time. Alternatively, a user may register his or her availability to classify documents with an active learning module 1660. Clinician 1010 may limit his or her availability, for example, by specifying a specific classification problem, certain document or EHR entry types, a specific document file extension or time during registration with active learning module 1660.

When a connection is made between active learning module 1660 and document review push process 1820 of FIG. 18, document review push process 1820 may be brought from the background into an active running state on client device 1520 of FIG. 15. Document review push process 1820 may receive data from active learning module 1747 (FIG. 17b) or 1497 of FIG. 14b and present the document and any other data (e.g., background data such as a prior related EHR entries for a same patient or other exemplary highly scored documents) to the clinician using an active learning interface such as interface 1520 of FIG. 15. In some embodiments, the transition from background to foreground process involves the presentation of a graphical user interface on the client device 1520. A similar background process technique may be used for pushing documents of an initial document set to clinician 1010 in order to allow clinician 1010 to make clinician coding decisions 955 (FIG. 9) for the documents of an initial document set.

In some embodiments, active learning module 1160 may send a message to clinician 1010, indicating that an EHR entry having assigned codes is ready for review. For example, active learning module 1660 may send a text message or email for the document that needs to be reviewed. An active learning interface may be presented to the clinician when clinician 1010 clicks or otherwise activates access to the EHR entry document or attachment presented in the message. A similar messaging technique may be used for allowing clinician 1010 to make clinician coding decisions 955 for documents in an initial document set.

In active learning mode, active learning module 1660 is able to receive clinician coding decisions 955 for the EHR entry (e.g., code class 1430 or subclass 1440 assigned to the entry). When a clinician coding decision 955 is received from the health care provider 1010, active learning module 1660 may update the classifiers using classifier generator 1640.

In some embodiments, instead of waiting for a clinician coding decision 955 (FIG. 14b) from a clinician 1010 (FIG. 15), active learning module 1660 (FIG. 16) may fork additional copies of classification process 1470 at step 1495 (FIG. 14b). These forked copies represent parallel document classification paths. Each forked copy of classification process 1470 will represent a prediction of the clinician coding decision 955 for the selected document as to a particular class or subclass of the classification problem. Thus, taken as a whole, forked processes represent the entire clinician decision space for the selected document with regard to all FIGS. 14a & 14b classes 1430 and subclasses 1440 of a classification problem. In some embodiments, original classification process 1495 (FIG. 14b) may be terminated or suspended after a document is selected.

While running, each forked classification process 1465 (FIG. 14b) may maintain local copies of classification dependent data (e.g., classifiers, priority queues, and/or document scores). Local copies of classification dependent data are considered local to a forked classification process 1470 (FIG. 14b) if they are not affected by another forked classification process 1165 during the period of time between forking classification process 1470 and receiving a user coding decision 1480, or between forking and the occurrence of a timeout waiting for user coding decision 1480 for the document.

Predicted classifiers and other data that maps onto a predicted user coding decision 1480 for the selected document for each forked classification process 1470 may be generated at step 1455 before running forked classification processes 1465. For example, the predicted classifiers used by each forked classification process 1465 will be modified by classifier generator 1455 using the method described according to a predicted user coding decision 1480. More specifically, for a two-class classification problem, forked process A may use local predicted classifiers updated by classifier generator 1455 to handle the situation in which clinician 1010 may find the selected document relevant (e.g., a member of class 1). Similarly, forked process B may use local predicted classifiers updated by classifier generator 1455 to handle the situation in which user 1475 may find the predicted classifiers not relevant to the selected EHR document. Forked process C may use local predicted classifiers that are not updated (corresponding to a situation where the active learning module 1660 potentially does not receive a clinician coding decision 955 within a specified time period, or the clinician coding decision 955 may not indicate whether or not a predicted code was not relevant to the selected EHR entry of the document). A similar forking system can be used for classification problems with additional classes 1430 and/or subclasses 1440.

Each forked classification process 1465 may, at step 1470, classify documents from document collection 1420 using their own local data copies (e.g., predicted classifiers, priority queues) substantially as if it is the only classification process that is running.

If a user coding decision 1480 is received or a specified time period is exceeded waiting for a user coding decision for the selected document, the active learning module 1660 may, at step 1485, terminate or suspend all forked classification processes 1470 which are inconsistent with the user coding decision 1480.

Global system data may be updated by active learning module 1660 at step 1485, for example, by copying or changing a pointer or reference to reflect the local data calculated by the forked classification process 1470 that matched the (un)received user coding decision 1480 for the selected document. In other words, the classification data generated by the forked process 1470 mapping to the correct prediction for user coding decision 1480 should be identified and propagated forward. After (non)receipt of user coding decision 1480, active learning module 1660 may start a new classification process 1450, or original classification process 1450 may be restarted. Started or restarted classification process 1450 may use classification data updated during step 1714. (FIG. 17a.)

In certain embodiments, classification system 1400 of FIG. 14a may nominate a classification process 1450 as a master classification process. For example, before active learning module 1660 is executed for the first time, a classification process 1450 may be created and nominated as the master classification process. During active learning, master classification process 1450 corresponds to the prediction that a received user coding decision 1480 for the selected document will not have the effect of modifying any classifier for a class 1430 or subclass 1440 or a timeout situation will otherwise occur. During active learning, as explained above, active learning module 1660 will fork other processes that represent a predicted user coding decision 1480 for the document (i.e., those responses that would require classifier generator 1455 to modify or update a classifier) for the document (e.g., relevant or non-relevant). For example, for a two-class classification problem, initially only a master classification process 1450 is running. After document selection, classification processes A and B are forked representing user coding decisions 1480 for the selected document of relevant and non-relevant, respectively. Classification process A updates its local classifiers (and other data as needed) to predict that the user will classify the EHR document as described in code ABC. Classification process B updates its local classifiers (and other data as needed) to predict that the user will classify the EHR document under review as described in class XYZ. Master classification process 1450 and forked processes A and B may run concurrently (on the same CPU core/processor or on different CPU cores/processors) or serially. If the user coding decision 1480 indicates ABC, process A is promoted to master classification process 1450 and its local classification data is propagated forward. If user coding decision 1480 indicates the EHR document is subject of code class XYZ, process B is promoted to master classification process 1450 and its local classification data is propagated forward. However, if the clinician response the EHR document is subject of code different than ABC or XYZ-master classification process 1450 remains the same and the classification data calculated by master classification process 1460 during concurrent or serial execution with forked processes A and B is propagated forward.

It will be appreciated that although the total number of EHR classification codes number into the tens of thousands, often the selection can be narrowed down to 1, two or 3 likely relevant codes. In some embodiments, instead of forking additional classification processes 1465 of FIG. 14b, active learning module 1660 may fork and maintain local predicted classification dependent data mapping to each predicted user coding decision 1480. Classification process 1400 is executed serially N-times for each document to be classified, where N represents the space of all predictions for user coding decision 1480. For example, for a two-class classification problem, data copy A may use predicted classifiers updated by classifier generator 1455 to handle the situation in which clinician 1010 may find the selected document relevant (e.g., in class 1). Data copy B may use predicted classifiers updated by classifier generator 1455 to handle the situation in which clinician 1010 may find the selected document non-relevant (e.g., in class 2), while data copy C may be an original classifier for a class or subclass. While awaiting user coding decisions 1480, the system may continue to classify documents, and classification process 1450 is executed using data copy A for a given document information profile. Thereafter, classification process 1450 is executed using data copy B for the same document information profile, and then classification process 1450 is executed using data copy C for the same document information profile. The process can be then be repeated for the next document in the collection while awaiting user coding decision 1480. The processing of data copies may be interleaved in any manner. For example, classification process 1450 may use data copy A to score a number of documents using their corresponding document information profiles, then may use data copy C to score a number of documents using their corresponding document information profiles, then use data copy A again before switching to data copy B. Although the order in which the data copies (e.g., A, B, C) are processed is unimportant, classification data (e.g., priority queues and scores) generated by each successive run of classification process 1450 should be maintained separately as with the other forking methods described above. As with the other forking methods, after user coding decision 1480 is received or a timeout occurs, the classification data generated by the correct prediction for user coding decision 1480 should be propagated forward and which may be displayed to the clinician. As with the other forking methods, this method can be extended to classification problems with any number of classes 1430 or subclasses 1440.

Active learning module 1660 may, at step 1495, determine whether a stopping criterion for the active learning process has been met. Determination of whether a stopping criterion is met is discussed further below. If a stopping criterion is not met, active learning module 1660 may continue back to step 1450 and select a further document. If a stopping criterion is met, active learning module 1660 may, at step 1497, classify the documents in the collection, for example, by comparing computed document scores with a threshold calculated during stopping criterion determination.

In some embodiments, active learning process 1660 may continue to run until all documents in document collection 1420 pertaining to the patient are processed and classified. In an alternative embodiment, active learning process 1660 may stop when a specified or determined number of documents have been classified by classification process 1495. In a further alternative embodiment, active learning process 1660 may stop when classification process 1495 has classified a specified or determined number of documents into a particular class 1430 or subclass 1440 or a particular set of classes 1430 or subclasses 1440. In a further alternative embodiment, active learning mode may stop when a specified number of user coding decisions 1480 have been received.

With limited processing power or a large enough document collection, it may be possible for a clinician to review a document and submit a user coding decision 1480 for the selected document before classification process 1455 or forked classification processes 1165 completed scoring and classifying each document of the collection. In this case, in order to allow real-time interaction with the classification system 1400B, it would be more efficient to suspend processing of the remainder of the document collection, and instead use the newly submitted user coding decision 1480 along with the new classifiers to be calculated from the newly submitted user coding decision. In certain embodiments, processing time may be allocated to forked classification processes based upon a confidence value associated with the prediction. For example, more processing time may be allocated to a forked classification process predicting that the selected document is relevant to a particular class or subclass when the selected document has a high score for that class or subclass.

For the above situation, in order to maintain high efficiency, the classification process 1400 or forked classification process 1470 may process the documents of the collection in a particular order, which may be determined at step 1460. For example, in this case, each successive iteration of the active learning process may be operating with incomplete information, meaning that scores and classification decisions for all documents of the collection will have not been calculated. More specifically, if selection of the next document for active learning may be based wholly or partly upon the subset of scores calculated by a forked classification process 1470 during an unspecified interval (e.g., between selecting a document and receiving a clinician coding decision), it is preferable to allocate processing time to those documents that will provide meaningful feedback for a subsequent document selection step 1485. For instance, after or in response to receiving a user coding decision, a next or further document may be selected using the score or priority queue data calculated by a forked classification process 1485 (or forked data copy) corresponding to the received user coding decision.

For example, classification process 1400, 1650 may process documents by order of rank in one or more priority queues. Alternatively, classification process 1650 may process the documents according to how closely scores for the document are to one or more calculated threshold values that are used to classify documents from the collection into classes 1430 or subclasses 1440, preferably using a calculated threshold that maximizes a stopping criterion. For example, documents may be processed from closest to farthest from the threshold (i.e., using uncertainty sampling). Different processing orders may be used for each forked classification process 1650 corresponding to predicted classifier. It will of course be appreciated after reading the above paragraphs, that the classification process may utilize the steps outlined in FIGS. 14a and 14b.

As a further alternative, in a different embodiment, classification process 1650 may combine one or more of these techniques to achieve a hybrid ordering scheme. In some other embodiments, documents are processed in a manner designed to complement document selection step 1450. For example, if document selection step 1450 will use uncertainty sampling on the next iteration, then classification process 1650 will also process documents according to uncertainty sampling. In certain embodiments, only a partial ordering is calculated or provided. For example, a certain number of documents may be ordered (e.g., the first 100,000 documents).

Alternatively, ordering may stop once a certain cutoff point is reached (e.g., the document score is below a certain threshold). As a further alternative, a number of documents may be ordered based upon an expected clinician review time for the selected document. For example, a review time may be estimated by the length of the selected document or by keeping a running average of intervals between received clinician coding decisions.

In certain embodiments, the processing order may be implemented as an input queue which is processed by the one or more forked classification processes. For example, enqueuing a processing order to a forked classification process may start or restart a forked classification process, which may continue to run until all elements of the queue are processed or the queue is otherwise empty. The input queue may be emptied in any number of ways. For example, processing a document listed in the input queue may remove an item from the input queue. In certain embodiments, an input queue may be emptied upon the receipt of a user coding decision.

Incomplete score and priority queue data may be augmented by score and priority queue data from an earlier iteration of active learning in order to calculate an approximate ranking for the priority queues. In order to acquire complete score and priority queue data for all documents of the collection, some iterations of active learning module 1660 and classification process 1650 may not be suspended when a new user coding decision 1480 is received.

The use of predicted classifiers and forking allows classification system 1400 to take advantage of the latency inherent in clinician review of the selected document. Thus, instead of performing calculations after the receipt of the clinician coding decision, forking allows classification system 1400 to have at least a set of partial calculations ready by the time the user coding decision 1480 is received. These partial results allow classification system 1400 to select a next document in an interactive and real-time manner. By allowing real-time interaction with a clinician 1010 through forking, a single user is able to quickly classify large document collections (e.g., tens of millions of documents). Furthermore, by cutting off processing with the receipt of new coding decisions, the classification system is both more efficient and responsive. Moreover, a single user approach requires less manpower and likely results in an increase in precision and recall. Precision and recall are increased given that the inconsistencies engendered by multi-user coding decisions are avoided. Additionally, a further increase in precision and recall can be achieved because a single user approach allows an expert on the matter to be employed in the classification effort rather than a team of novices or other persons unfamiliar with the subject matter.

The product of the Applicant's disclosure is an electronic health care record (EHR) that includes the text of the prior clinician's examination observations and diagnosis, as well as treatment and treatment responses, as well as the results of clinician ordered tests. The tests may include but are not limited to laboratory blood tests, electrocardiograms, X-rays, CT scans, etc. Further, the EHR is enhanced by incorporation of entries of relevant ACD, CPT or other recognized codes associated with the diagnosis, treatment or tests.

### Clinician Specification & Selection

The clinician may be prompted to specify left or right knee, etc. This would disclose the sub-class. The health care provider may be prompted to initiate a search using codes M17, M24, M25, M67 or M94.

In FIG. 15 illustrates one embodiment of a system of this disclosure. The system may be comprised of one or more devices 1520. Each device has an input device 1528 that is accessed by a clinician 1010. The clinician enters text describing the examination of a patient. It will be appreciated that the multiple devices may be receiving input regarding separate individual patients. In one example, the text entry includes a diagnosis of the patient's health condition. The entered text is displayed 1522 for the clinician. The text entry is also entered in the processor 1524. The processor includes the one or more code protocols. In response to the text entry, the processor suggests and displays at least one code entry from a protocol. The suggested code is selected by the content of the health care provider's text entry, e.g., diagnosis. The device includes a storage device 1526. It may be a transitory of volatile storage. The clinician may accept at least one of the suggested code entries. The text entry and the accepted code(s) are stored in memory. The clinician may initiate the search function from the device 1520.

It will be appreciated that the disclosure incorporates the text of the health care provider's notations, along with the related code entry. In an embodiment, the EHR entry may also include the code descriptor or identifier. Other prior art systems only incorporate “extracted facts” into the EHR. Utilizing the “extract facts” method, the text descriptions of the prior art systems may be redacted from the full entered text into an abbreviated structured format. This can eliminate nuances or significant details that were contained in the clinician's full and complete notation. In a preferred embodiment of the Applicant's disclosure, such detail remains accessible as part of the EHR. This disclosure includes retaining the clinician's notations in a non-abbreviated and non-structured format.

It will be further appreciated that the clinician may utilize terminology (slang) that is not included in the protocol of the text descriptors. However, the input device display will alert the clinician of the discrepancy when no or inapplicable codes are suggested. The clinician will have the opportunity to insert the correct code or revise the text entry to more closely match the vocabulary used by the code descriptors.

Continuing with FIG. 15, the system and device may be connected to a local network 1530. This network may be confined to a specific medical office or to a local network of a single hospital. It may be networked to multiple branches of the hospital. The entered text and code, being an entry to a patient's Electronic Health Record (EHR), can be transmitted to central server 1540 containing a non-volatile or non-transitory storage component 1544 and processor 1542 for retention. The storage component 1544 may comprise the data collection 1420 of FIG. 14a above. In another embodiment, the updated EHR can be retained by a shared but dispersed/distributed network wherein identical copies of the EHR (as may be updated) are retained. The multiple copies of the ESR can be compared to ensure no single copy is inappropriately modified.

The storage of the EHR entry in the storage component 1544 of the server 1540 frees the memory of the device 1520 to continue processing further entries and suggest additional codes correlated to each text entry.

The application discloses an embodiment of the system for providing a document classification platform within a server. FIG. 15 illustrates a simplified classification scheme. The EHR records 1420 containing the clinician's entries may be communicated in real time to the classification module of the processor 1670 illustrated in FIG. 16 discussed above. The text of each entry is reviewed and evaluated in the classification system 1400 and the entries are correlated into pre-existing classification classes 1430 (categories) and subclasses 1440 (subcategories). Initially the classification system is the code protocol and the classes and subclasses are as defined in the code descriptors. It will be appreciated that the code descriptors are modified as the system learns the alternate terms used synonymously by some health care providers as contained in multiple EHR entries. The multiple text terms of the multiple health care providers become associated and are correlated with specific codes as code identifiers.

### EHR Security

It is yet another goal of the disclosure to provide security to the patient's EHR, i.e., ensure that the contents of the EHR are not tampered with nor that there be any unauthorized disclosure of the EHR contents. This may require that the clinician be identified as a person or entity authorized 1120 to have access to the patient's EHR prior to the clinician being allowed to create data or information that will be added to the patient's EHR. (See FIG. 11) Therefore it is a goal of this disclosure to provide an authentication process. This may be particularly applicable where the clinician is a member of a larger entity, e.g., hospital, or network of entities.

In one embodiment, the clinician may be subject to a two factor authorization process wherein encryption may be required as one step and a second step requiring authentication by entering a user name and password. As discussed in further detail below, this disclosure includes teaching of an EHR electronic data entry form for entry of the text notes of observations and diagnosis. The electronic data entry form requires identification of the patient as well as the identity and authorization of the clinician. The form also allows the entered data, e.g., 11502a of FIG. 11, to be instantly converted to an EHR search request without the clinician having to duplicate entry of any information. The EHR search request form can be auto-populated with the information appearing on the EHR data entry form. See FIGS. 11, 17a & 17b.

This feature will allow the search for past diagnoses and treatment relevant to the current observed patient conditions to be initiated prior to completion of the diagnosis and formulation of a treatment plan or ordering of tests. Also the ability to auto populate the search request form avoids additional time as well as transcription errors.

### Devices

The classification scheme 1400 of FIG. 14a (see also FIG. 9) may be implemented in conjunction with the system illustrated in FIG. 15. As shown therein (FIG. 15), a plurality of client devices 1520 may in communication with one or more servers 1540 over a network 1530. The network may be any type of network such as one that includes the Internet, a local area network, a distributed network, a wide area network, an intranet, etc. Users 1010 are clinicians or support personnel that may access a code protocol for the purpose of conducting, overseeing or performing the desired code protocol classification with the diagnosis/treatment/test description. Clinicians 1010 may utilize devices 1520 to communicate with the server 1540. The user devices 1520, as well as server 1540, may be configured to communicate via wired or wireless links, or a combination of the two. It will be appreciated that the storage devices 1526 of each of the three user devices 1020 illustrated in FIG. 15 may contain identical copies of each EHR, thereby creating a distributed network. In another embodiment, the server 1540 may be connected to a network of servers, each containing identical copies of multiple EHRs in a distributed network.

In one embodiment shown in FIG. 15, user devices 1520 may represent a desktop computer, laptop computer, cell or smart phone, tablet device, or other type of computing device operating in conjunction with a server 1540. The server may be part of a network as elsewhere described. The server communicates with multiple user devices and communicates all code protocol modifications created from the machine learning function to each device. Each of the user devices 1520 may be equipped with one or more computer storage devices 1526 (e.g., RAM, ROM, PROM, and/or SRAM) and one or more processing components 1524 (e.g., a central processing unit, CPU or microprocessor) that are capable of executing computer program instructions. These instructions may include the machine learning functions.

Continuing with FIG. 15, the device processor may, in one embodiment, perform the classification/sub-classification functions. It will be appreciated that any modification of the code descriptor may be communicated to the central server 1540 wherein the modification may be transmitted to all client devices 1520. Computer storage device 1526 of the server is preferably a physical, non-transitory medium.

Continuing with FIG. 15, any of the user devices 1520 may further include a display 1522 that is capable of rendering an interface such as the ones described in subsequent sections and one or more input devices 1528 (e.g., keyboard, microphone, camera, video camera, scanner, joystick, remote control device, and/or touchscreen). Clinicians/users 1010 may manipulate interfaces rendered on the display 1522 using the input devices 1528 to communicate with the server 1540. It will be appreciated that embodiments of these displays are illustrated in FIGS. 10, 11, 12 & 13.

In some embodiments, server 1540 comprises one or more mainframe computing devices that execute a web server for communicating with client devices 1520 over the Internet. The storage medium on server 1540 can store applications or software code that is configured to assist users 1010, e.g., clinicians, in creating entries pertaining to diagnosis/treatment/tests.

Specifically, server 1540 may be configured to provide document classification services (illustrated in FIG. 15) utilizing one or more code protocols to users 1010 via an interface displayed on user devices 1520. It will be appreciated that the clinician devices may display entered text, suggested codes and code descriptors as shown in FIG. 11, for example. Returning to FIG. 15, server 1540 may be configured to perform the steps in any of processes of FIGS. 1 through 8 and may further be configured to transmit data for displaying the interfaces shown in FIGS. 10a-10f, 11, 12 or 13. For example, referencing FIG. 15, server 1540 may cooperate, e.g., assist in code correlation, with a user device 1520 to present suggested code entries and code classifiers from the processing unit 1542 of the server to a clinician 1010, and to display a clinician interface that permits the clinician to enter codes relevant to an EHR entry.

Further, the storage devices 1526 or 1544 (FIG. 15) may be internal or external physical media on which an EHR document 1420 of FIG. 14a, or a portion thereof may be stored, imported or accessed. Storage devices shown in FIG. 15, 1526 or 1544 may be located on yet another storage medium or facility, such as a data storage warehouse, server farm, cloud storage facility, or file hosting service.

One useful feature provided by this system relates to the fact that a number of classification processes may continue to run on server 1540 (FIG. 15) or processor 1670 of FIG. 16) while awaiting a user coding decision (described further below) for the suggested codes and code classifiers. This useful feature, which permits continued document classification at the multiple user devices while awaiting a user response, is facilitated by a unique classification forking scheme that prioritizes the processing of documents, thus allowing real-time interaction between classification system 1400 of FIG. 14a and FIG. 14b and clinician 1010 of FIG. 15. Another useful function performed by server 1540 relates to the server's ability to update the code modifiers in real time so that all users (including users via a network) are benefiting from the increased accuracy between the vocabulary of the health care providers and the codes of the code protocol.

It should be noted that the system in FIGS. 15 & 16 are merely meant to demonstrate one embodiment of an operating environment and should not be construed as limiting in any manner whatsoever. The particular configuration in FIG. 15 can be altered in numerous ways without departing from the principles of this disclosure. For example, it should be noted that the functionality of server 1540 in FIG. 15 may be carried out by a plurality of servers. Likewise, although the FIG. 15 depicts a single server 1540 connected to three client devices 1520, any number of servers 1540 and client devices 1520 may be utilized with classification system 1400 of FIG. 14a, and classification system 1400 may be configured in a variety of different ways (e.g., in a distributed computing environment, cloud-based environment, block chain environment, distributed network and/or client-server environment).

Furthermore, while FIG. 15 illustrates a plurality of client devices 1520 in communication with a server 1540 over a network 1530, it should be recognized that the functionality provided by server 1540 to client devices 1520 may be performed locally on each of client devices 1520. For example, client devices 1520 may utilize an application and/or server that executes locally on client devices 1520 to perform the functions of server 1540. Thus, any functionality of server 1540 which is described herein can alternatively be implemented by a client device 1520, and vice versa.

The system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, touchscreens, etc.) may be coupled to the system either directly or through intervening I/O controllers.

Network adapters may also be coupled to the system to enable the data processing system to become coupled to other data processing systems or remote printers or storage devices through intervening private or public networks. Modems, WiFi, cable modem and Ethernet cards are just a few of the currently available types of network adapters.

Embodiments described herein may be hardware-based, software-based or may comprise a mixture of both hardware and software elements. Thus, while the description herein may describe certain embodiments, features or components as being implemented in software or hardware, it should be recognized that any embodiment, feature or component that is described in the figures or description herein may be implemented in hardware and/or software. In certain embodiments, particular aspects are implemented in software, which includes but is not limited to firmware, resident software, microcode, etc.

### Initialization Interface

Initialization interface 101 of FIGS. 19 & 20 allows the clinician 1010 of FIG. 15 to initialize classification system 1400 (FIG. 14a). Initialization interface 101 may allow clinician 1010 to create or specify classes 1430 and subclasses 1440 for a classification problem. Clinician creation or specification of classes 1430 and subclasses 1440 (FIG. 14a) may include the ability to annotate or attach a description of each class 1430 or subclass 1440. For example, user 1010 (FIG. 15) may describe a class 1430 or subclass 1440 as being related to a patient medical issue such as a “knee sprain” or may describe class 1430 or subclass 1440 as being “radiographic examination of a knee” or “X-ray image of left knee”. (As the above examples should make clear, the code protocol and code modifiers may pertain to testing.) In some embodiments, the health care provider 1010 need not specify classes 1430 or subclasses 1440 for a classification problem. It will be appreciated that the classification may be performed by the classification system 1400. As discussed, the classification system may comprise a CPU or similar programed with the appropriate code protocols and capable of machine learning. Creation or specification of classes 1430 or subclasses 1440, will, in one embodiment, begin with the up loading of a code protocol. As illustrated above, the code protocol will have code descriptors. The descriptors will provide subject identity for each code. Also, in some embodiments, the alphanumeric organization of the code protocol identifies the class and subclasses. (As already mentioned, the learning function of the system will allow further descriptive value to be added to or modification of the code descriptors.) The classification system 1400 may initialize or create data constructs associated with the system (e.g., priority queues and/or classifiers). The classification function shown in FIG. 14a may be performed by the processor 1524 or 1542 of FIG. 15.

Returning to FIG. 19, initialization interface 101 also allows the clinician 1010 of FIG. 15 to define or categorize portions of the EHR document 920 of FIG. 9. For example, initialization interface 101 may display an interactive display window (see for example FIGS. 10a or 11) on the clinician device 1020 suitable for creating classifications or subclasses, e.g., diagnosis, description of specification of treatment to be performed, treatment results, tests order, test results, etc. It will be appreciated that the user device 1520 of FIG. 15 may contain a display 1522 and input interface 1528. The initialization interface may allow the health care provider to select past patient information, e.g., selected index portions of the patient's EHR files or directories, from local or network storage. (See FIG. 15, File storage 1526.) This step may be initialized by the clinician utilizing the data inputted onto the display, i.e., the health care providers (draft) diagnosis, etc. Files (excerpts from past patient EHR entries) selected by a clinician 1010 may be contained in the document collection 1420 of FIG. 14 or 920 of FIG. 9. (Of course, the EHR is property of the patient and the patient may directly provide access to the requested EHR records. The health care providers being authorized custodians of a copy of the patient's EHR, i.e., the EHR document 1420.)

In a preferred embodiment, the entries of the clinician, descriptions of procedures or treatment, test results, etc., are added to the patient's EHR 1420.

Note also that EHR records may be added to the system collection by coupling a device or cable with server 1540 or client device 1520. For example, after inserting one or more external storage devices (such as a USB, SATA, or Thunderbolt drive), network attached storage (NAS), or similar device into a corresponding port on client device 1520 or server 1540, the files or documents contained thereon are added to document collection 1420. Although the documents within the collection 1420 illustrated in FIG. 14 are often refenced as a single patient's EHR, the collection 1420 may, in other embodiments, contain the EHR records of multiple patients. The health care provider will identify the correct EHR by patient identifiers e.g., name, DOB, social security number, etc. Again, see FIG. 10a or FIG. 11 as containing embodiments of this identification step.

See FIG. 16. Initialization interface 1022 may also allow the clinician 1010 to indicate settings for active learning module 1660 It will be appreciated that active learning is the machine learning discussed above. For example, initialization interface 1022 allows clinician 1010 to determine whether or not classification system 1000, 1100 will use active learning. Initialization interface 1022 may also allow clinician 1010 to specify other clinicians or experts 1010 for receiving documents or notifications during active learning and/or review of the initial document sets.

Active or machine learning may utilize a database of medical dictionaries, journals, texts, articles that can bridge and correlate the vernacular of an individual clinician to the accepted medical terminology incorporate within the code protocol, particularly the code descriptors. This database may contain supporting material and editorial comments that accompany the promulgated code protocol as well as past protocols, i.e., descriptors of ICD-9 and ICD-10 and “cross walk” guidance and indices. This database may be contained in the server 1540 illustrated in FIG. 15.

In an embodiment, the clinician 1010 may create a text entry of a patient diagnosis. The text entry may be entered into the client device 1020. In one embodiment, the text will appear in pane 1002a of FIG. 10a. The text will appear as the user enters the text via the Input Device 1028 of FIG. 15. The text entry may specify or name the diagnosis, e.g., “appendicitis”. The health care provider may create a separate entry describing the treatment to be performed or a description of a treatment result. The entry pertaining to treatment results may be part of a subsequent consultation or in entries justifying a patient hospital discharge order. The entry may appear in a separate pane 1002b.

The text entry may be reviewed by the processor 1524 of the clinician device 1520. The processor may contain the code protocol as well as the rules or program for correlating the text entry to one or more codes, utilizing the code descriptors (contained in the code protocol). In this function, the server is acting as the classification system.

In one embodiment, the clinician may determine and/or select a class 1430 and/or subclass 1440 to be associated with the text entry (or portion of the text entry). As mentioned above, a classification problem (correlation of the clinician's text entry to one or more codes) may require machine evaluation of any number of classes 1430 and subclasses 1440. The entry may pertain to a past diagnosis/treatment/test. Such past event may already be subject to one or more of code classifications contained in the patient's EHR 1420. These past code entries may have utilized modified or enhanced code descriptors (code modified class description). The processor may evaluate these past indexed entries in suggesting one or more code entries to the clinician via the client device display 1522. In one embodiment, the suggested code classifications may be displayed with the coded descriptor or modified class description, i.e., class identifier.

The possibly relevant portions of the EHR pertaining to past diagnosis/treatment/test may be temporarily stored in the storage device 1526 of the clinician device. This storage may comprise transitory or volatile memory.

It will be appreciated that all or a portion of the informational documents pertaining to past patient diagnoses, etc., contained in the EHR may be a member or a non-member of any number of classes 1430 or subclasses 1440 of a classification problem (e.g., the documents may be relevant or non-relevant to any number of classes 1430 and/or subclasses 1440 of a classification problem). Stated differently, a portion of the EHR may have no relevance to the classification problem of the current health care provider. Thus, the clinician 1010 may submit multiple user coding decisions indicating whether the informational documents in the initial EHR are to be associated with any particular class 1430 or subclass 1440 arising from the current diagnosis, etc.

Based upon the coding suggestions (containing the code descriptors or modified class identifiers) provided to the clinician based upon the processor evaluation of the clinician's text entry, the clinician may be prompted to revise or edit the entry to enhance its descriptive value. The clinician may be prompted to provide greater specificity in his/her entry. This may reduce the number of displayed codes. It will be appreciated that at this initial stage, the processor may be associating the contents of the original entry with the code descriptors. This may be viewed as similar to a keyword search. However, the system may select suggested codes utilizing knowledge of past selected code entries, including codes containing a clinician modified class descriptors (class identifier) for the suggested code. This would be machine learning.

FIG. 16 illustrates a further embodiment of the server 1540 of FIG. 15. The initial document set generator 1620 may execute a keyword search operation on the past EHR entries contained in the document collection 1420. The search may use the Wumpus Search Engine (developed at the University of Waterloo) running the BM25 (OKAPI) algorithm or other type of search engine. For example, the EHR document 1420 may be provided to the search engine for analysis (e.g., the documents may be uploaded or otherwise made available), and a clinician 1010 may be permitted to search the document collection providing keyword queries to the search engine. The search engine may return a subset of the EHR document according to the specified search algorithm which satisfy the query provided by the clinician. The returned search documents or subsets may be displayed upon the display screen 1522 of clinician's device 1520. Each of the entries of the EHR in the subset (or a portion thereof) may be ranked based on how closely the EHR document portion or utilized code matches the user's query. The subset of documents (or portion thereof) returned in response to the user's query may be displayed to the health care provider as relevant patient history. For example, only those EHR entries above a certain rank may be added to the display. In another embodiment, the code and modified code descriptor may be displayed as a suggested code entry. Naturally, the form of user query (and the results) may be dependent on the classification problem to be solved.

### Code Protocol Amendment

In one embodiment, the system may derive and create amendments to the code descriptors based on health care provider's coding decisions. In a further embodiment, these proposed modifications to the original code descriptors of the code protocols may be collected in the system memory for evaluation. If identical or similar modifications are frequently suggested and collected by the system, the code descriptor utilized by the system may be amended. (As previously discussed, the ICD code is periodically updated. ICD-9 was updated in approximately 2015 to ICD 10. ICD-10 is to be replace with ICD-11 in approximately 2022.) The step of collecting proposed code descriptor changes for evaluation and comparison may allow the code descriptors, as thus code usage, to more closely align with the intent and meaning of the clinicians. The step of collecting groups of proposed changes may prevent the code from being modified based upon unsupported or unjustified code selections of one or few clinicians.

Referring to FIG. 16, in some embodiments, the document information profiles derived from the user coding decisions may include a vector or array derived using a 4-gram technique. The description of the document information profile generator 1630 and/or a classifier generator 1640 provides with regard to how the user coding decisions 955 of FIG. 9 may be utilized in creating document information profiles and code identifiers.

Generally speaking, the clinician's entry (text description) into the display 1522 of clinician device 1520 (FIG. 15), as shown in the windows (panes) 11502a, 11502b, etc., of FIG. 11, may be utilized by the classification system to determine whether or not a code should be suggested or whether a code entered by the clinician should be accepted by the system without modification of the clinician's entry. These concepts are discussed in further detail below.

Further, in one embodiment, the system's initial document set generator 1620 may communicate and coordinate with document manager 1610 to access and display indexed portions of the patient's existing EHR 1420 (FIG. 14a). In one embodiment, the clinician may control activation of this feature. In an embodiment, for example, the clinician's entry of a diagnosis, etc., may prompt the suggestion of possibly relevant codes. Simultaneously, the EHR may be reviewed by the processor for similar codes or similar text. These portions of the EHR may be displayed 1522 of FIG. 15 to the clinician using the device 1020. In this example, the contents of the displayed documents may be utilized by the clinician in creating a diagnosis or treatment plan.

In certain embodiments, e.g., when merging active learning into a single phase, generation and review of initial code descriptors is not performed and instead classification system 1400 of FIG. 14a may use an iterative active learning approach.

FIG. 11 illustrates an embodiment of the disclosure comprising a suggested format for the display screen 1100 of the clinician device 1020 (see FIG. 15). The clinician may enter examination notes 11502a utilizing the device input interface 1028. In one embodiment, the left-hand screen 11501a begins to become populated with suggested or prompted code entries based upon the text of the clinician entered in 11502a. The suggested code entries include both the code and the code descriptor. The disclosure utilizes machine learning to select possibly applicable codes from the code protocol. The device utilizes learned past selected or entered codes made by a clinician wherein the clinician's text description (or text description of a separate clinician) was similar to the current entry. Relevant portions of past EHR entries may be displayed for the clinician's consideration. This display of relevant portions of a past EHR entry may appear in window 11502a or 11502b or, in another embodiment, in a separate window. The display of the past EHR entries may be a function of the search option shown in FIG. 11.

### Patient Identity and Data Authenticity

Also disclosed in the embodiment illustrated in FIG. 11 is the requirement that the patient be identified 1110 by name, DOB, and other criteria such as social security number, assigned number from the entity. The entity may be the health care provider's doctor's office, the emergency room of a hospital, an outpatient clinic or similar. In one embodiment, each entity may be part of a patient information network. Each entity may have a network identifier or number. The participants in the network may share patient information if authorized by the individual patient.

To facilitate efficiency of the clinician/health care provider's time and effort, the health care provider may input his/her observations, etc., 11502a electronically. In one embodiment, the information may be entered on a format similar to that shown if FIG. 11. First the patient is identified by name, DOB and/or other identifying information 1110. To ensure the authenticity and validity of the health care provider's observations, diagnosis, etc. recorded in window 11502a, the clinician may first be required to “sign in” 1120 to the network (it being appreciated that the network may be a single health care provider's office or expanded network as suggested above). The clinician must enter a user name and password. If the network recognizes the user name and the correct association of the name with the password, the health care provider's note, diagnosis, etc. may be entered onto the illustrated form. The network will communicate the suggested codes and code descriptors that will appear in window 11501a of FIG. 11.

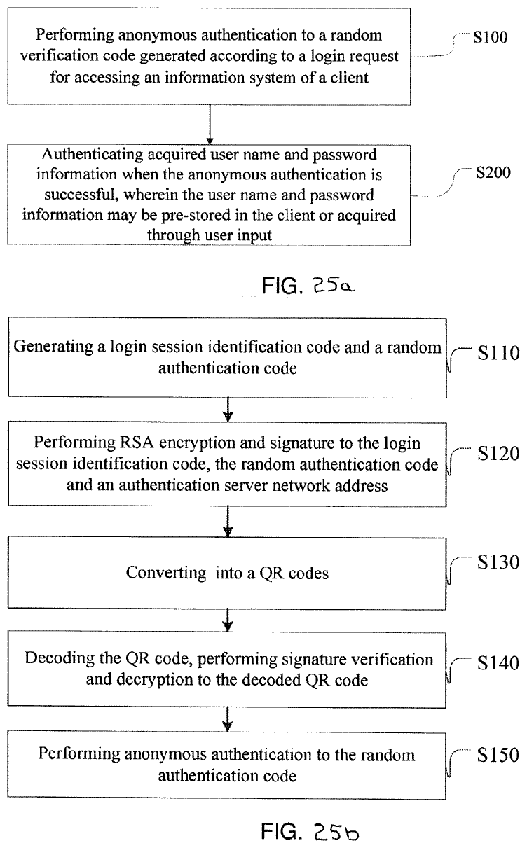

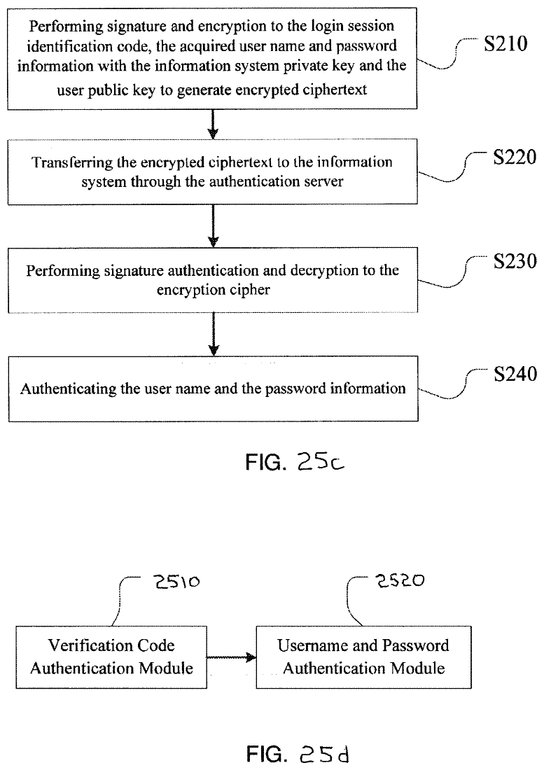

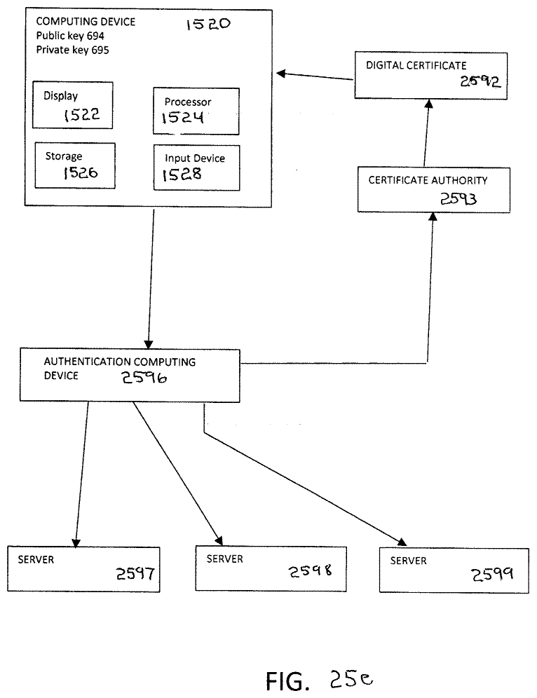

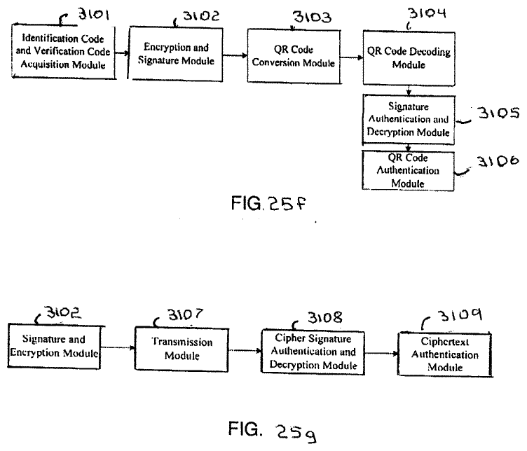

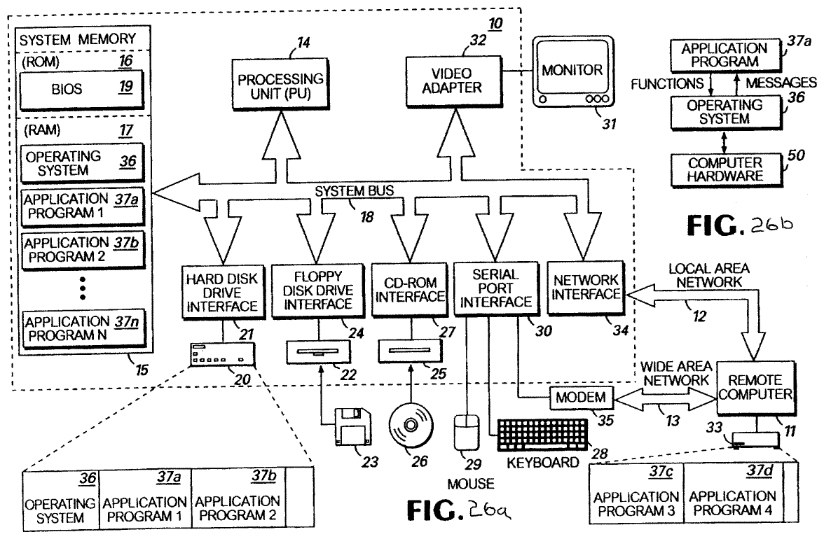

The integrity of the data, i.e., the validity of patient data and the data accuracy, can also be ensured in other embodiments utilizing block chain and distributed network techniques. These applications may also use private and public key technology as described below in relation to FIG. 25e.

Further, the clinician may request 1130 that the other entities of the network also provide relevant information that they possess pertaining to the identified patient. The information may be contained within the patient's EHR or known to one or more of the networked entities. This request may be easily made by the clinician by indicating such a request in the space provided within the form utilized for entry of the clinician's notes pertaining to the patient. The clinician may request information from one or more identified entities or from all the participating entities of the network. In one embodiment, the search request will automatically be submitted utilizing the search function initiated by activating the search tab “Search” 1131. While awaiting the result of a requested search, the clinician may continue accessing the EHR data entry screen. The network may be comprised of entities similar to the network of the Health Information Exchange for South East Texas, http://www.ghhconnect.org.

As described elsewhere, the search may be conducted utilizing the suggested or selected health care codes. These codes appear in the window 11501a adjacent to the window 11502a containing the clinician's text notes. In a further embodiment, the clinician may be able to modify the search request, using information supplied for the past EHR entries furnished in response to the first request

Note that FIG. 11 discloses an embodiment wherein it is possible for the user to continue writing notes wherein the complete text extends beyond the size of the window 502a. The continued text can be viewed by several methods. As illustrated in FIG. 11, each window, e.g., 11501a, 11501b, 11502a, 11502b, may contain a scrolling device 505 that can vary the contents of the window. The complete text can be viewed in the window by other methods such a holding a mouse key and moving the cursor up or down within the window. In another embodiment, the contents of a window may be varied by a configuration utilizing the vertical scrolling or movement of a user's finger across the surface of the window or display screen 1022 of the user device 1020 discussed in FIG. 15.

It will be appreciated that the device 1020 may display 1022 images such as images of hand written entries contained in a past EHR entry created by the clinician or other authorized individual. X-ray or MRI or CT images may also be displayed. The device may also contain a feature allowing a single pane to be expanded to occupy the entire display screen, thereby facilitating accurate understanding of the image.

FIGS. 9 & 13 illustrate another embodiment of an interface 550 which allows the clinician 1010 to navigate between the clinician's EHR entry and the coding protocol with the purposes of allowing the clinician 1010 to make prompt coding decisions 551 and capture those coding decisions with the relevant EHR entry.

In this screen display 590, the past relevant EHR entry is displayed. The EHR entry is selected by the processor based upon the selected code entries or text entries of the current clinician. In the embodiment shown, the clinician may scroll through the past EHR entry utilizing arrow keys 570 or the arrow keys contained within the control panel 540. It will be appreciated that the text entries may be used in the search. This will allow productive access to the past EHR entries that have not been previously annotated or indexed utilizing the code protocols.

### Data Entry Validation

As indicated above, only an authorized user, i.e., clinician such as a physician, nurse, physician's assist, nurse practitioner, etc., may enter data that may be included as part of a patient's EHR. As further indicated above, the identity of the clinician may be required to be authenticated 1120 prior to the entry of the data. See FIG. 11. Authentication may be achieved by various methods. All such methods are included within the scope of this disclosure.

In another embodiment, the document source 920 (FIG. 14a) may need to be authenticated in order that the current clinician may trust the authenticity of the received documents.

In the embodiment illustrated in FIG. 11, the clinician is required to enter an established user name (first authentication information) and corresponding password (second authentication information). See the sign in spaces 1120 of FIG. 11.

FIG. 25a is a flow diagram illustrating a method allowing the clinician to obtain access to an EHR.

The advantages of this embodiment include that user login, i.e., the clinician, utilizes a two factor protocol comprising two stages: a first stage, including anonymous authentication S100 and a second stage, including identifying information authentication S200, in which the user needs to provide the user name and password for authentication. It will be appreciated that entering the first stage may not require the user name and password to be provided and only the random verification code is acquired to be verified by a direct anonymous authentication method to allow the access request to move to the second stage. The authentication of two stages can effectively reduce the risk of the user login information leakage and improve security.

In step S100, the recipient of the access/search request may be a single entity or a broad network of clinicians. The random verification code acts as challenge information for anonymous authentication.

In step S200, the acquired user name and password information is authenticated when the anonymous authentication (first stage) is successful, wherein the user name and password information may be pre-stored by the recipient entity or acquired through user input.

It will be appreciated that there are a variety of ways to implement the anonymous authentication to the random verification code in step S100. FIG. 25b illustrates one example. FIG. 25b above illustrates the method steps for performing anonymous authentication of the random authentication code, i.e., the first stage of the two stage authentication.

Beginning with the first step S110, login session identification code and a random verification code is generated according to a login request of the clinician for accessing the information system. The login session identification code is temporary and unique.

In the second step S120 asymmetric cryptographic algorithm (RSA) is performed, generating an encryption and signature to the login session identification code, the random verification code and an authentication server network address with an information system private key and a user public key.

It will be appreciated that an authentication server may be utilized to provide a function of registration of a clinician, e.g., a hospital participating in a network. This clinician or EHR custodian, i.e., recipient, is receiving a request from a current clinician. This current clinician may be an emergency room doctor. This request may be activated after an authentication application is installed by the recipient. The recipient should first be registered in the authentication server, and the recipient may create a link with the authentication server at any time through linking to the authentication server network address for authentication. The known techniques in the prior art can achieve the RSA encryption and signature, and the transmission of the authentication information is more secure and reliable when transmitted with the help of the asymmetric cryptographic technique.

### Encryption of Device and EHR Data

It will be further appreciated that asymmetric cryptography, also known as public key cryptography, uses public and private keys to encrypt and decrypt data. The keys are simply large numbers that have been paired together but are not identical (asymmetric). One key in the pair can be shared with everyone and is called the public key. The other key in the pair is kept secret; it is called the private key. Either of the keys can be used to encrypt a message; the opposite key from the one used to encrypt the message is used for decryption. It will be appreciated that encryption is the method by which plain text or any other type of data is converted from a readable form to an encoded version that can only be decoded by another entity if they have access to a decryption key. It will be further appreciated a key is a piece of information (a parameter) that determines the functional output of a cryptographic algorithm. For encryption algorithms, a key specifies the transformation of plaintext into ciphertext, and vice versa for decryption algorithms. Keys also specify transformations in other cryptographic algorithms, such as digital signature schemes and message authentication codes.

Stated further, asymmetric cryptography is any cryptographic system that uses pairs of keys: public keys which may be disseminated widely private which are known only to the client. This accomplishes two functions: authentication, where the public key verifies that a holder of the paired private key sent the message, and encryption, where only the paired private key holder can decrypt the message encrypted with the public key.

In a public key encryption system, any person can encrypt a message using the receiver's public key. That encrypted message can only be decrypted with the receiver's private key. To be practical, the generation of a public and private key-pair must be computationally economical. The strength of a public key cryptography system relies on the computational effort (work factor in cryptography) that would be required to find the private key from its paired public key. Effective security only requires keeping the private key private; the public key can be openly distributed without compromising security.

Asymmetric cryptography systems often rely on cryptographic algorithms based on mathematical problems that currently admit no efficient solution, particularly those inherent in certain integer factorization, discrete logarithm, and elliptic curve relationships. Public key algorithms, unlike symmetric key algorithms, do not require a secure channel for the initial exchange of one or more secret keys between the parties.

Because of the computational complexity of asymmetric encryption, it is usually used only for small blocks of data, typically the transfer of a symmetric encryption key (e.g. a session key). This symmetric key is then used to encrypt the rest of the potentially long message sequence. The symmetric encryption/decryption is based on simpler algorithms and is much faster.

In a public key signature system, a person can combine a message with a private key to create a short digital signature on the message. Anyone with the corresponding public key can combine a message, a putative digital signature on it, and the known public key to verify whether the signature was valid, i.e. made by the owner of the corresponding private key. Changing the message, even replacing a single letter, will cause verification to fail. In a secure signature system, it is computationally infeasible for anyone who does not know the private key to deduce it from the public key or any number of signatures, or to find a valid signature on any message for which a signature has not hitherto been seen. Thus the authenticity of a message can be demonstrated by the signature, provided the owner of the private key keeps the private key secret.

The third step S130 of this example embodiment involves converting the encrypted and signed login session identification code, random verification code and authentication server network address into a QR (Quick Response) code. The known QR code conversion software or program in the prior art can achieve the QR code conversion. It will be appreciated that the transmission of the authentication information is more secure and reliable with the help of the asymmetric cryptographic technique.

FIG. 25c is a flow diagram illustrating a process of authenticating information of a user name and a password according to one embodiment of the present disclosure.

In another embodiment, a request to login to the system includes first successfully performing anonymous authentication to a random verification code and second, authenticating the clinician name and password.

Another embodiment of the disclosure described above FIG. 25d includes a device for providing access to the EHR system including a verification code authentication module 2510 and user name and password authentication module 2520 connected to the verification code authentication module. The verification code authentication module is configured to perform anonymous authentication to a random verification code generated according to a login request for accessing an information system of client; and the user name and password authentication module is configured to authenticate acquired user name and password information when the anonymous authentication is successful.

In one embodiment, the user name is converted and a credential that includes a public key may be extracted from the user name. The password may be decoded and may be decrypted based on the public key extracted from the user name. The user name and password may be authenticated by comparing the decrypted password to the extracted credential. If the comparison results in a match, the computing device may be authenticated.

In some embodiments, the user name comprises supplemental information concatenated to the retrieved credential. The supplemental information may comprise a time stamp generated at the time the user name is generated. The time stamp may be extracted from the user name. After the user name and password are compared, the time stamp may be verified in order to complete authentication. The time stamp may be verified by comparing the extracted time stamp to previously received time stamps for that computing device. If the extracted time stamp is different from the previously received time stamps for the computing device, the extracted time stamp may be confirmed.

An example system of authenticating a computing device is described further below with reference to FIG. 25e. In some embodiments, the system may comprise computing device 1520 (e.g., user device from FIG. 15), previously established credential 2592 (e.g., digital certificate, PKI, etc.), trusted authority 693 (e.g., certificate authority), public key 694, private key 695, authentication computing device 2596, directory 2597, database 2598, and computing device 2599 (e.g., servers). In some embodiments, the device seeking authentication may be any suitable computing device (e.g., client computing device, peer computing device, etc.) that seeks authentication in an authentication system.

In some embodiments, authentication computing device 2596 and computing device 1520 (1520 of FIG. 15) implement a username and password authentication policy. In this embodiment, first authentication information may comprise a user name and second authentication information may comprise a password. Computing device 1520 may provide one or more credentials via the user name and password to authentication computing device 2596 and the one or more credentials may be authenticated by authentication computing device 2596. In an example, computing device 1520 and authentication computing device 2596 are associated with an Electronic Health Record system. In another example, the authentication computing device 696 has no prior knowledge of computing device 1520.

In some embodiments, computing device 1520 (FIG. 25e) and authenticating computing device 2596 implement a public key cryptography system. For example, public key 2594 and private key 2595 may comprise a set of asymmetric keys. When data is encrypted using private key 2595, public key 2594 may be used to decrypt the data. For example, a digital signature for computing device 1520 may comprise hashing data prior to transmission, e.g., based on a 256-bit secure hash algorithm (SHA), and then encrypting the digest of the hash with private key 2595. The digital signature may be decrypted using public key 2594. Any other suitable hashing algorithm (e.g., SHA-224, any hash algorithm published by the National Institute of Standards and Technology, etc.) may be used.

FIG. 25f discloses an identification module and verification code acquisition module 3101 in communication with an encryption signature module 3102 and QR code conversion module 3103. The QR code conversion module 3103 is in communication with the QR code decoding module 3104 as well as the signature authentication and decryption module 3105 and QR code authentication module 3106. FIG. 25g illustrates the signature and encryption module 3102 also in communication with a transmission module 3107, then a cipher signature authentication and decryption module 3108 and cyphertext authentication module 3109.

A hash algorithm is a function that converts a data string into a numeric string output of fixed length. The output string is generally much smaller than the original data. A hash function is any function that can be used to map data of arbitrary size to data of a fixed size. The values returned by a hash function are called hash values, hash codes, digests, or simply hashes. Hash functions are often used in combination with a hash table, a common data structure used in computer software for rapid data lookup. Hash functions accelerate table or database lookup by detecting duplicated records in a large file. They are useful in cryptography. A cryptographic hash function allows one to easily verify that some input data maps to a given hash value, but if the input data is unknown, it is deliberately difficult to reconstruct it (or any equivalent alternatives) by knowing the stored hash value. This is used for assuring integrity of transmitted data, and is the building block for HMACs, which provide message authentication.

A cryptographic hash function is a special class of hash function that has certain properties which make it suitable for use in cryptography. It is a mathematical algorithm that maps data of arbitrary size to a bit string of a fixed size (a hash) and is designed to be a one-way function, that is, a function which is infeasible to invert. The only way to recreate the input data from an ideal cryptographic hash function's output is to attempt a brute-force search of possible inputs to see if they produce a match or use a rainbow table of matched hashes. The input data is often called the message, and the output (the hash value or hash) is often called the message digest or digest.

The ideal cryptographic hash function has five main properties:
i. it is deterministic so the same message always results in the same hash;
ii. it is quick to compute the hash value for any given message;
iii. it is infeasible to generate a message from its hash value except by trying all possible messages;
iv. a small change to a message should change the hash value so extensively that the new hash value appears uncorrelated with the old hash value;
v. it is infeasible to find two different messages with the same hash value.

### Authentication

FIG. 17a provides an illustration of the steps of an embodiment wherein the clinician initiates a search 1702 of past EHR entries that may pertain to the same medical issue now being confronted by the current clinician. The clinician may first designate the entities 1703, 1704, 1705 to receive the search request. See also the categories 1130 illustrated in FIG. 11, i.e., Search locations and Search scope. Note the clinician may create a custom search which allows specified codes or text to be searched. The search will be initiated by the clinician selecting the “Search” button. 1131.

FIG. 17a further illustrates an embodiment for authenticating the party requesting the search by inserting a first factor identification 1706. Note in the example provided, the requestor, i.e., user or clinician, may make 3 attempts to supply a correct authenticator (password or identifying credential) 1708, 1709. If the requestor's authentication is accepted, the requestor may be subjected to a second layer authentication 1710. This may comprise two factor authentication and require a second level of acceptance 1711. This may be an authentication code separately texted or emailed to the requestor. Again, the requesting party may have 3 attempts to submit an acceptable second factor identifier 1712, 1713. Upon acceptance of the requestor's credentials, the search may be performed. The search results may be encrypted and returned to the requesting party (clinician) 1714.

As illustrated FIG. 17a illustrates the user authentication steps 1706-1714. Also see FIG. 111120 requiring entry of a user ID and password. The authentication factors may be entry of the user name and password. The system may, in another embodiment, employ a two factor authentication process 1710. Note that the receiving/disclosing entity (“Recipient Network”) may encrypt the patient information transmitted to the health care provider 1714.

FIG. 17b expands the steps of one embodiment wherein the Recipient Network discloses responsive or relevant EHR entries to the requesting health care provider. First, the requesting clinician may be authenticated 17141. The patient is next identified 17142 and a determination made whether the recipient network possesses an EHR for the patient. In the illustrated embodiment, the clinician may provide a declaration that the requested information is required for continued health treatment of the patient 17143. This document may satisfy the Recipient Network that a patient consent is not required. It will be appreciated that this step will comply with the Health Insurance Portability and Accountability Act (HIPAA). If the Recipient Network is the custodian of any patient EHR documents containing information responsive to the search request (whether by text entry or codes listed in the search request) the responsive information can be provided to the clinician 17145, 17146, and 17147. If the Recipient Network requires patient consent to the release of information, the authenticated clinician is to be notified 17148.

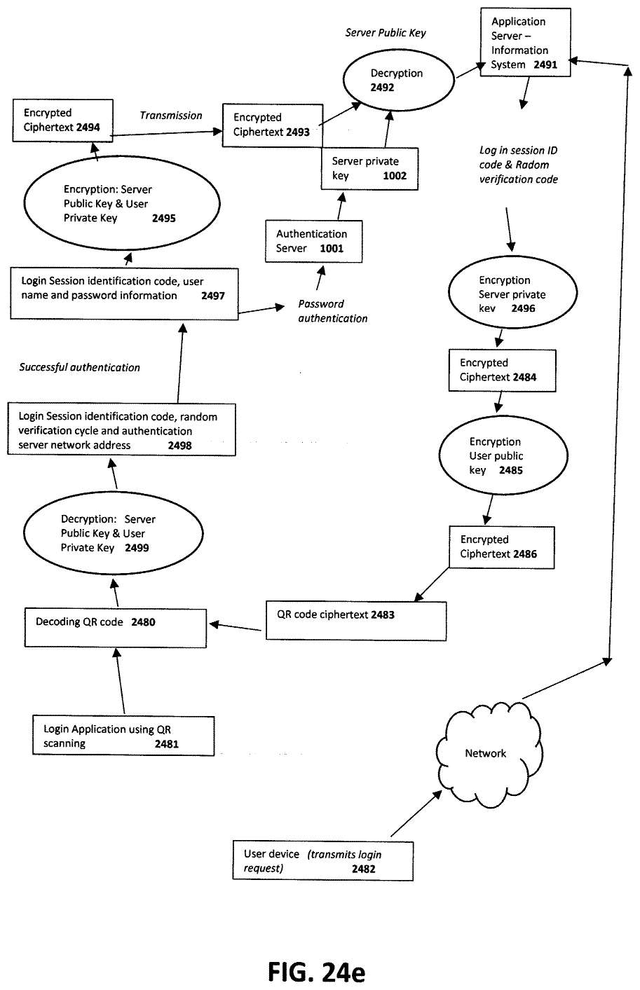

FIGS. 24a through 24d illustrate an authentication process that may utilize a QR code 2401

FIG. 24a illustrates a log in interface 2400 containing an authentication code 2401, along with webpage (e.g., “www.singou.mo”) 2410 corresponding to a unique URL and a bookmark column 2420 that includes a bookmarklet 2422 (shown as “[+]SINGOU”). The authentication code 2401 comprises a QR code that is generated in response to retrieval of a session ID from a gateway server. The authentication code 2401 includes information such as a session ID and the URL that are to be obtained by a mobile device when reading (e.g. scanning) the authentication code 2401.

FIG. 24b illustrates a user interface 2401 at a mobile device in accordance with an example embodiment. The user interface includes a block 2430 for inputting a user name, a block 2435 for inputting a password, and one or more triggers or buttons 2436, 2437, and 2438.

As an example, when no user name and password associated with a URL that is extracted from an authentication code displayed by a client device is found in a list of URLs, user names and password in the mobile device, a user inputs a user name and a password into blocks 2430 and 2435 respectively.

The one or more buttons perform a variety of functions. For example, in response to activation of the button 2436, the mobile device stores a URL, a user name and a password associated with the URL into the list in the mobile device. In response to activation of the button 2437, the mobile device denies login to a computer system. In response to activation of the button 2438, the mobile device generates a counter value associated with a URL for voting the URL.

FIG. 24c illustrates a process for processing a user name in accordance with some embodiments. The process begins at step 2440, where the received user name is converted into PEM format or any other suitable format. In some embodiments, the user name will be received in the desired format (e.g. PEM), and the converting will be unnecessary. The process of FIG. 24c may move from step 2441 to step 2442, where a credential (e.g., digital certificate) is extracted from the user name. In an example, the user name may have been received as a concatenation of supplemental information and a digital certificate, and the extraction may comprise separating the digital certificate from the supplemental information. The extraction may comprise additional steps, such as converting a format for the user name, decoding the user name, separating the credential from other appended data, etc.

From step 2441 the process of FIG. 24c may move to step 2442, where supplemental information is extracted from the user name. In an example, the user name may have been received as a concatenation of supplemental information and a digital certificate, and the extraction 2443 may comprise separating the digital certificate from the supplemental information. The extraction may comprise additional steps 2444, such as converting a format for the user name, decoding the user name, separating the supplemental information from other appended data, etc.

The process of FIG. 24d illustrates an embodiment of converting a received password. In an example, the received password may have been digitally signed by a computing device, and the conversion may include decrypting 2445 the digitally signed password.

FIG. 24e illustrates the clinician initiating a login request from the clinician device 2482 links to the application server 2491 over the network, and sends a login request, and the application server returns a user login interface 2481. The application server generates a login session identification code and a random verification code for the login request. The application server performs RSA encryption and signature to the login session identification code, the random verification code and authentication server network address with the server private key and the user public key, to generate an encrypted ciphertext.

The application server converts the encrypted ciphertext 2486 into a QR code 2483 and display the QR code on the user login interface at the client; the login application installed in the user scans the QR code through a camera device and decodes the QR code 2480.

The login application decrypts the decoded QR code with the server private key and the user public key 2499 to obtain the login session identification code, the random verification code and authentication server network address 2498. The login application links the authentication server to perform anonymous authentication to the random verification code, and the authentication server returns a response message;

The application server determines whether the anonymous authentication is successful according to the response message, and if the anonymous authentication is successful 2498, the application server informs the authentication server to start the second stage of authentication 2497. The login application 2481 then queries the user name and the password information stored in the user, and the user name and the password information can be also acquired by user input.

The login application performs encryption and signature to the login session identification code, the user name and the password information with the information system private key and the user public key to generate a new encrypted ciphertext 2486.

The login application links the authentication server network address to transfer the new encrypted ciphertext to the authentication server. The authentication server then transfers the new encrypted ciphertext to the application server over the network.

The application server performs signature authentication and decryption to the new encrypted ciphertext with its own server private key 2496 and the user public key 2485 to obtain the login session identification code, the user name and password information; and the application server authenticates the user name and the password information, returns a successful login message when the authentication is successful and the login procedure is completed that the user's login is successful, and returns an error message when the authentication is unsuccessful.

Compared with the prior art, the above method and device for information system access authentication has the following advantages.

1. The user's login of the information system includes two stages: a first stage, including anonymous authentication, in which it is not required to provide the user name and password, and it is only required to acquire the random verification code and verify the random verification code with a direct anonymous authentication method; and a second stage, including identifying information authentication, in which the user need to provide the user name and password for authentication. The authentication of two stages can effectively reduce the risk of the user login information leakage and improve security.

2. Two-factor authentication (i.e., QR code authentication and user name and password authentication) is required when a user logs in the information system, which combines the QR code technology and the asymmetric encryption technology to make the transmission of the authentication information more secure and reliable.

3. With the application software which facilitates the scanning of the QR code and the input of the password, the security is improved while the user's operation experience is also improved.

A health care provider may be able to rapidly conduct a search of a patient's EHR to gain, in real time, the patient's health history. This history can be obtained via an electronic search and disclosure request that (i) identifies the patient, e.g., contains the patient's name, DOB, SSN, etc., (ii) confirms the patient's consent to the disclosure, (iii) identifies the requesting entity, a secure transmission receiving address, (iv) any cryptographic security measures such as a public key, and (v) the scope or substance of the of the request, e.g., a text statement of requested information such a past “injury, laceration, strain, sprain, twist, soreness or swelling of the left knee, or a series of ACD or CPT codes applicable to the listed types of injury or trauma. The EHR could be searched for the text “laceration of left knee”, “left knee strain”, “left knee twist”, etc. or combination of these and other text terms. In another embodiment, the EHR can be search using ACD, CPT or other codes that pertain to injury of the left knee. In some embodiments, the requesting health care provider may use an expanded set of codes for an enhanced capture of relevant information.

It will be appreciated that the patient's consent to a request for all or a portion of the EHR may be obtained at the time of the initial signing of consent to treatment documents. The patient's signature can be digitally copied.

The health care provider's request can be directed to one or more identified entities. Alternatively, the request may be directed to unspecified entities. In yet another embodiment, the request can be directed to an organization representing numerous health care providers. An example of such an entity could be Greater Houston Healthconnect https://www.ghhconnect.org.

### EHR Security and Encryption

Referencing FIG. 4, in one embodiment, the health care provider enters patient ID (e.g., name, DOB, SSN, etc.) 401 and data (diagnosis/treatment/test data) into the health care provider's device (sometimes referred to as the “user's device”) 402. This is data obtained in a patient encounter, e.g., emergency room visit. As more fully described elsewhere, the entry of text by the clinician pertaining to patient diagnosis, etc., into the user device may prompt codes, e.g., ICD, CPT, etc., being displayed 403. The clinician may enter codes based upon the suggestion of the user device or enter separate codes 404. The data, optionally appearing on the display screen of the user device, may be transmitted from to a server connected to a network 406. This first server may be at the clinician's office, clinic, hospital, laboratory, etc. Upon direction of the clinician, making a search request from the device, the server may initiate communication with one or more EHR repositories 405. The data having been entered by the clinician may include a user ID. The user ID may be transmitted to the EHR repository, along with a previously established password. The step may ensure that the search request, containing patient information, is being received from a proper source. Utilizing this information an EHR repository may initiate a search of its records for information pertaining to the identified patient 406.

In another embodiment, the patient information may be provided additional security. The EHR may require that the requesting server provide authentication information. This information may comprise a certificate from an independent certification authority, e.g., VeriSign, etc.

In another embodiment, the patient data/search request may be encrypted. This encryption may utilize a private key. In one such embodiment, the first server may transmit a public key to the EHR repository. This public key may allow the recipient to decrypt the search request information. It will be appreciated that the search request information will include alphanumeric code information that identifies factors of the clinician's assessment or diagnosis of the patient. As stated above, the alphanumeric code may be selected in conformance of a code protocol such as CPT or ICD, etc. The search request may also include the text of the clinician's assessment or diagnosis. The search request may also contain additional code entries as suggested information that should be included in the search. These additional codes may have been suggested by the system at the time the clinician inputted his/her assessment or diagnosis.

Each EHR repository receiving the search request may electronically search for records pertaining to the identified patient 406. Each repository will further seek to identify information within its electronic records that pertains to the listed codes of the search request (both codes pertaining to the diagnosis, treatment or test or codes additional codes that have been listed in the request) 407. Also the search may include text correlated to the text entries that may be included in the search request. Again, the health care provider may also suggest key words to be searched within the EHR repository information for the identified patient. Responsive data is transmitted from the EHR repository 408 to the user device of step 2401.

The relevant documents based upon code or text, which may in one embodiment include test results (images such as MRI or x-rays) are retrieved. The information may be transmitted back to the requesting party (the clinician).

In order to assure the clinician that the information received is from an authentic source and that the information can be relied upon in the treatment of the patient, the system may require that each responding EHR repository provide certification from a certificate authority, e.g., VeriSign, etc. Further, the patient's privacy may be maintained by each EHR repository encrypting the data to be transmitted using a private key. The certificate furnished to the clinician may include a public key that allows the device (or receiving server) to decrypt the EHR records responsive to the search criteria.

It will be appreciated that the patient's records of relevant past medical treatment will now be available to the clinician, e.g., an emergency room physician. These electronic records will have been searched and transmitted with little delay. The transmission of information may avoid duplication of testing or treatment, including unnecessary costs and expenses.

The apparatus initiating a search of an EHR repository may comprise an electronic device containing a processor, memory, user interface, display screen and programing. The device allows a health care provider to enter information into the device pertaining to a patient encounter, the device displays the entered information and the device suggested codes and code descriptors selected by the device in response to the entered health care provider information. This device is also illustrated in FIGS. 10a-10f, 11 and 13.

The EHR repository will comprise one or more servers in communication with a network such as an Internet or local area network (LAN). The server memory may contain electronic health records (EHR) received from multiple health care entities such as physicians, clinics, hospitals, laboratories, etc. The records may have been originally created in electronic format or may be scanned images of notes created in a patient encounter such as physical examination (perhaps resulting in a diagnosis) or treatment procedure (drug therapy, physical therapy, counseling, surgery, etc.) or testing (radiation, MRI, echocardiogram, blood testing & results, etc.).

The records will contain patient identifying information such as patient name, date of birth, social security number, etc. The records may be annotated with alphanumeric codes derived from one or more code protocols such as Current Procedural Terminology (CPT) or International Classification of Diseases (ICD). The coding annotation may have occurred as part of the initial recording of information, or subsequently added in a coding step (perhaps related as part of the health care provider's invoicing procedure). In another embodiment, the coding may occur at the time the data is received and recorded within the EHR repository. In another embodiment, the records my later be annotated with the coding. The coding annotation may occur at the EHR repository by merging patient billing records with the health care information, i.e., the records of the patient encounter. In another embodiment, the EHR repository may utilize medical coders to electronically assign the appropriate alphanumeric code(s) to the received records.

The health care provider may instruct the device to conduct a search of the patient EHR wherein the device communicates information to a remote server (i.e., a computer connecting one or more computer accessed EHR repositories) wherein the information comprises patient identity and alphanumeric codes wherein the codes have been assigned from one or more code protocols. The search request may also contain word elements or test classifications, e.g., “INR” for measurement of blood coagulation properties.

In an embodiment, the search request transmitted to the EHR server will contain at least one alphanumeric code specific to the patient's observed condition or diagnosis. The codes may also be specific to treatment or medical procedures that have performed for the benefit of the patient. The codes may also pertain to tests, etc., that have been conducted for the benefit of the patient in the course of medical treatment. These codes will be matched with codes contained in the EHR records. The search may include entries related to similar codes utilizing machine learning described in the related applications.

The codes will have been selected from a code protocol (ICD, CPT, etc.) wherein each code describes and pertains to a particular health care condition or treatment or test, etc. Another coding protocol is the Healthcare Common Procedure Coding System (HCPCS). Yet another protocol is the Diagnostic Related Grouping (DRG) codes.

In an embodiment, the health care provider identity and authorization/passcodes, identity of the device initiating the search request, and device authorization/authentication information may be required as part of the search request communication. In one embodiment, the clinician (requesting party) may utilize a pre-established user name and password. In other embodiments, patient's EHR data may be transmitted to the requesting party (clinician) using asymmetric encryption and a “public key infrastructure” (PKI) as discussed below.

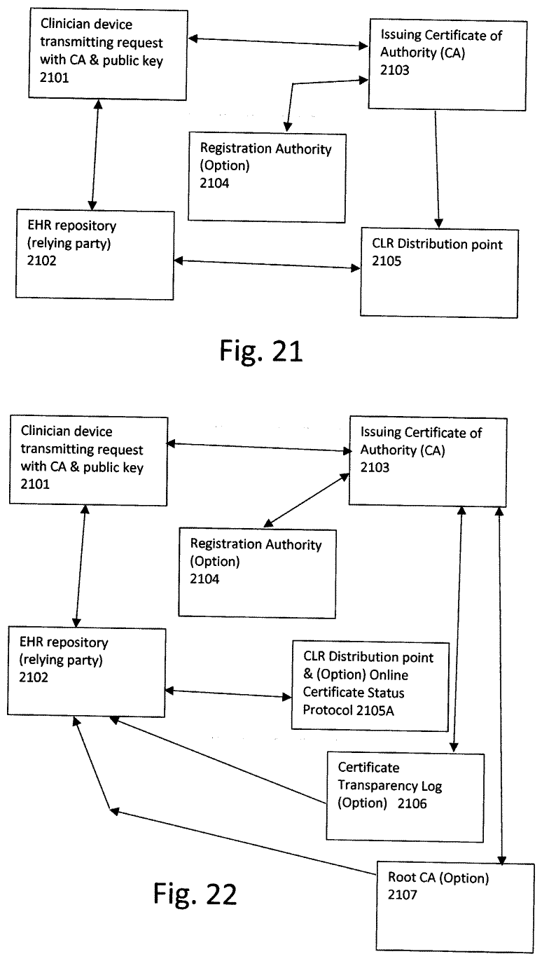

Applicant's FIG. 21 illustrates a public key infrastructure. The health care provider may transmit the text and selected alphanumeric codes to an EHR repository 2101. To assure the EHR repository that the request is from a legitimate/authorized health care provider (and not a phishing transmission), the request is accompanied by a certificate from an issuing authority (CA) 2103. The search request may be encrypted by a server in communication with the health care provider's device. The request may therefore be transmitted with a public key that will allow the EHR repository (relying party) to decrypt the search request 2102. Note that the authentication of the certificate subject party (clinician) may be performed by a registration authority (RA) 2104 in communication with the certificate authority 2003. It will be appreciated that the certificates may be limited for a duration in time. Also certificates can be revoked for reasons such as the clinician's security having been compromised. Revoked certificates can be tracked by a CLR distribution point 2105. The CLR distribution point may communicate to the relying party (EHR repository) 2102 that a proffered certificate from the subject (clinician) 2101 has been revoked.

FIG. 22 illustrates an additional embodiment of a public key infrastructure shown in FIG. 21 wherein a segregated and more secure root CA 2107 is used to issue certificates to the CA 2103. Also a certificate transparency log 2106 may be in communication with the EHR repository 2102. Further, the CLR distribution point may include an online certificate status protocol to expedite the EHR repository's inquiry whether the certificate proffered by the requesting party 2101 is valid.

It will be appreciated that the EHR repository wants to be assured that the request for patient data is being received from an authorized entity. Conversely, the requesting party wants assurance that the received data is valid data from the repository.

In an embodiment, the clinician device must be authenticated. This may require authentication utilizing the device MAC address and prior assignment of an authentication code. See FIG. 19, item 1909. Note also that the step of authenticating the user device of step 1909 may be performed at the time the request is initially received. See step 1903 & 1904. It may require utilization of a Session Initiation Protocol (SIP).

Note that in the above embodiment, the device must also be authorized to send and receive information to the EHR repository. The device may be identified from its Media Access Control (MAC) address. The MAC address may have been previously recorded as an authorized address for transmission and receipt of data. There may also need to be initiation of a Session Initiation Protocol (SIP) which may utilize a proxy server. The requestor (health care provider) may also be required to be authorized 1904. This may be performed utilizing a user ID and password. It may also be performed utilizing a certificate authority issued by a third party CA. See FIG. 21, item 2003.

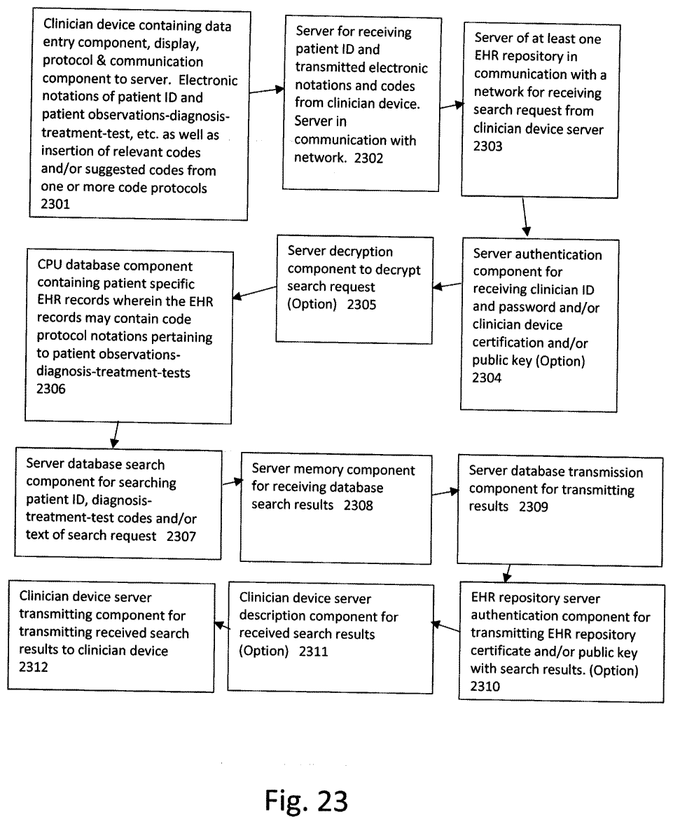

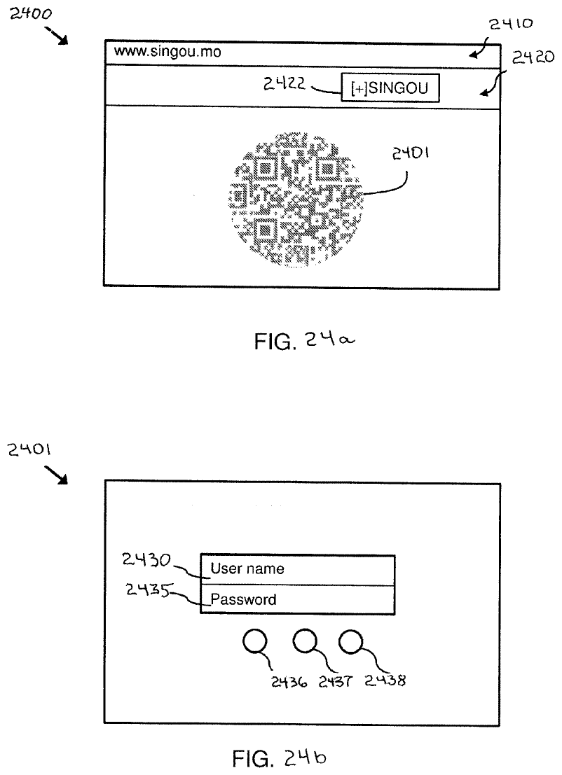

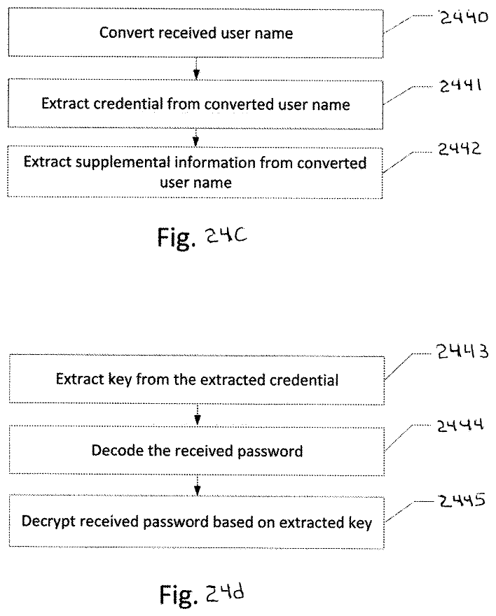

In another embodiment, a system of components is used to record diagnoses, codes and conduct searches of a patient's EHR. FIG. 23 illustrates this system wherein a clinician's device contains data entry components, display, processor with at least one code protocol and communication components to a server 2301. The server of the system receives patient ID information and the transmitted clinician notations and suggested codes 2302 wherein the server is in communication with a network. A separate server of at least one EHR repository is also in communication with the network 2303. This server may receive search request from the clinician server 2202. In an option, the EHR repository server includes an authentication component 2304. This authentication component may further include authentication of the clinician ID and password and or a public key. Further, there may be an optional decryption component to decrypt a received search request 2305. The receiving server containing a CPU data base of patient specific EHR records with code protocol notations pertaining to past patient observations, diagnosis, treatment and tests 2306. The receiving (EHR repository) server including capability to search patient ID, diagnosis, treatments and test codes as well as optionally text of search request 2307. The server further includes a memory component for receiving search results 2308 and transmitting components for transmitting results 2309 to requesting server. The repository server may optionally include authentication components for transmitting EHR repository certificate and public key with search results 2310. The clinician requesting server may optionally include decryption components for the received search results 2311. The clinician device server transmitting component for transmitting the search results to the clinician's device 2312.

In another embodiment, only the requesting party (clinician) is required to be authenticated. To maintain the confidentiality of patient EHRs, the requesting party and EHR may utilize asymmetric cryptography with public key infrastructure and creation of a private key and public key. The public key infrastructure (PKI) may include a certification authority (CA), as well as a registration authority (RA) working with the CA, root certificate authority, etc.

It will be appreciated that the certificate (issued by a third party CA) assures the one party (relying party) to an information exchange, e.g., the clinician or EHR repository, that the requesting party or EHR repository (subject party) is whom they say that are. Each certificate has a unique serial number, may be valid for a defined term, and identifies the certifying authority. Certificates can be cancelled and replaced. Cancelled certificates are identified on a public Certification Revocation List (CRL). An Online Certificate Status Protocol (OCSP) may also be utilized. Further the PKI may include a certificate transparency log identifying all of the issued certificates.

In one embodiment, the EHR repository may create a private key and related a public key utilizing an algorithm. The private key is confidential and retained by the EHR repository. The private key will be utilized to encrypt data (portions of the patient's EHR responsive to the request). The requesting party may have a copy of the public key that can be utilized to decrypt the EHR data. It will be appreciated that the EHR repository's public key may be widely disseminated among clinicians that may seek access to the electronic health records within the repository.

In another embodiment, the public key received from the EHR repository may be authenticated utilizing known protocols. The public key may be transmitted with a certificate assuring that the key is from the repository. In this embodiment, the repository may be referred to as the “subject” of the certificate and the requesting party is the “relying party”. The authentication will assure the clinician receiving the data and decrypted with the received public key is in fact from the EHR repository. Stated differently, the clinician will know that the public key used to decrypt the encrypted patient data is in fact the public key of the EHR repository.

The EHR repository can utilize its private key to encrypt the responsive data. See FIG. 20, item 2003 & 2004. The EHR repository, i.e., relying party, can decrypt the search request with the transmitted public key 2005. The EHR repository can search for the existence of records pertaining to the identified patient 2006 and, if records are located, the records may be searched using the alphanumeric codes contained within the search request 2007. The EHR repository can transmit its response along with its own Certificate Authority to the requesting party and the search results 2008. The requesting party can decrypt the response with the EHR repository public key contained with the Certificate Authority. This public key may also be stored on the requesting party's device.

In another embodiment, the user device communicates directly (and simultaneously) with one or more EHR databases. This would be distinct from a wheel and spoke model. In other words, the device is not required to communicate with a central clearing house that may comprise the combined databases of multiple entities, e.g. hospitals, clinics, physician's offices, etc.

The requesting party, i.e., clinician, will preferably have a 1. pre-established user name and a 2. Passcode. This should constitute 2 factor authorization and allow the subject (EHR repository) to trust the origin of the request, i.e., the request is from a clinician requesting patient information for the purpose of medical treatment of the patient.

The device used by the requesting party, e.g., a tablet computing device, smart phone, desktop CPU, etc., will have an established and unique MAC address. The MAC address will further allow the EHR repository to trust the validity of the information request. The method may also utilize public key infrastructure (PKI).

In a separate embodiment, for each device communicating, i.e., initiating a search request, with the repository database, authentication or legitimacy of the requesting party may be required by the repository server. This can be in addition to or in lieu of the user name/password combination. In this embodiment, each user would have to be authorized by the repository of EHR. This could also be in addition to or in lieu of separate authorization of the entity server, e.g. prior authentication of a MAC address.

In one embodiment, both the device and the user must be authorized.

In an embodiment, there are multiple users, each possessing their own CPU device, e.g. a tablet device. Multiple tablets could be individually connected a server. The server may be located an entity, medical office, clinic, hospital, laboratory, imaging facility, etc. This “entity server” may communicate with the EHR repository/database server.

In one embodiment, the entity server would require authorization from each user (user name, password, and/or use of a PKI structure). In turn the server would have to be authenticated to the receiving repository of the EHR.

As mentioned above, in another embodiment, each user device would communicate with one or more repositories.

Upon authentication of the requesting party (and in some circumstances, authentication of the device), the device or entity server transmits the search request. Each search request would contain the patient identity, as well as (i) codes, e.g., selected ACD, CPT, etc., codes and (ii) specified search terms. The specific search terms may be specific laboratory test classification, e.g., INR for blood clotting capability, physical ailment or anatomical parameter, e.g., knee ligament. The search codes would be the codes, e.g. alphanumeric codes correlated to medical conditions (diagnoses), treatments or tests appearing on the display of the clinician's device. (The clinician can enter the code(s), utilize the suggested codes, or select among the suggested codes for the scope of the search.) The specified terms may be selected from the health care provider's diagnosis or treatment notes, etc.

It will be appreciated that there are multiple code protocols. In one embodiment, the device (used by a clinician) may contain multiple code protocols. The entry of a draft diagnosis or observed condition may result in the device displaying not only the text entered by the clinician but also alphanumeric codes correlated to the patient diagnosis, etc., but also the appropriate or suggested codes of several code protocols. This feature will allow all entries within the EHR repository to be searched notwithstanding the repository containing entries utilizing or indexed with differing protocols. The function of supplementing codes appearing on the clinician device in response to the clinician's text entries may be performed by a server in communication with the clinician device. For example, if the user device displays and transmits codes selected from the ICD-10 protocol, the server may correlate the ICD-10 code to the counterpart CPT code, etc. Both the ICD-10 and CPT codes will therefore be searched in the records of the EHR repository.

For example, text entry of “torn right knee ligament” would, under the ICD-10 code provide the following suggested codes:
a. S83.401A Sprain of Unspecified collateral ligament of right knee
b. 0MQN0ZZ Repair of right knee bursa and Ligament, Open Approach
c. 0MQN3ZZ Repair right knee bursa and ligament, Percutaneous Approach
d. 0MQN4ZZ Repair right knee Bursa and ligament, Perc Endo Approach

The CPT protocol would list the following for the same “torn right knee ligament search:
a. 27405 Repair, primary, torn ligament and/or capsule knee: collateral
b. 27407 Repair primary torn ligament & Capsule knee Cruciate
c. 27409 Repair, primary, torn ligament and/or capsule, knee; collateral and cruciate ligaments

The repository would first search its database for an EHR for the relevant/identified patient. If an EHR was not located, a message could be communicated to the user device stating that fact. If an EHR was located, the repository (server) could optionally search using the criteria set forth in the search request. If there were any extra-ordinary information disclosure restrictions (beyond that required of HIPAA), the disclosure requirements could be communicated to the clinician device. The clinician would have to attempt to comply with the extra-ordinary requirements in order to proceed further.

Note that the system subject of this disclosure does not require that the search be of a specific format. The search will identify the patient and the relevant diagnosis/treatment/test codes of the search. The search may also include text entries that may be searched. The combined code and text entries will provide enhanced search results.

The repository may require the clinician device to provide confirmation that the requested information is required for treatment of the patient (thereby exempting the disclosure of patient information from any consent requirement under HIPAA).

Each physician's office, clinic, hospital and laboratory may create their own EHR repository. Preferably, an individual EHR repository may be linked to multiple other repositories. In some embodiments, the inter linked EHR repositories may comprise a distributed network wherein each repository maintains a ledger of each entry made to a specific patient's EHR. This constitutes a block chain of EHR records, thereby ensuring the authenticity and accuracy of the information within the EHR. This distributed ledger structure may be facilitated by the authentication and confidentiality procedures utilized in the

Each EHR entry would, of course, identify the patient and the date of encounter (examination/treatment/test, etc.). In an embodiment, the electronic entry will contain the notes or observations of the clinician along with suggested or selected billing codes. Similarly, a physician's notes of treatment would also contain the codes suggested or selected. If the clinician's services were invoiced to a third-party payor or invoiced to the patient wherein billing codes were assigned, the billing codes could be included in the clinician's notes. These codes contained in the patient records of the EHR repository may be used as an index to expeditiously search for information related to the clinician's search request. Again, the search request may contain codes of multiple code protocols that are relevant to the clinician's diagnosis, etc. Again, it must be remembered that the clinician device may automatically suggest billing codes based upon the contents of the clinician's notes (or ordered tests, the test results collated with the billing code and order).

In a further embodiment, the EHR repository may include invoices containing the codes associated with the invoiced diagnosis/treatment/testing.

Accordingly the preceding discloses a tool such as a tablet computer or similar that can record a clinician's notes of patient systems or treatment responses. The method of utilization of this tool creates an entry into a comprehensive patient electronic health record (EHR). The tool and method include correlating the clinician's electronically recorded notes to accepted medical diagnosis and treatment coding protocols. The disclosure teaches how the tool may facilitate clinician use by providing suggest autocompletion of entries. The disclosure also teaches how the tool or device can correlate the clinician's entries to accepted code protocols, including use of the code descriptors. The clinician may accept suggested diagnostic treatment codes or modify the notations to prompt a new or modified slate of suggested codes.

The disclosure thereby teaches the integration of the codes into the patient specific EHR. The disclosure teaches how the EHR may be searched utilizing the codes to identify EHR entries of relevance and interest.

The EHR records, annotated with a searchable code or index is also subject of this disclosure.

The tool of the disclosure may also be utilized for the display of search results, including images such as recorded X-ray images.

The disclosure further teaches the tool and methods requiring entry of authentication information for the protection of patient privacy of EHR. These methods include but are not limited to two factor authentication or private/public key combinations.

### Auto Correct & Autocomplete System

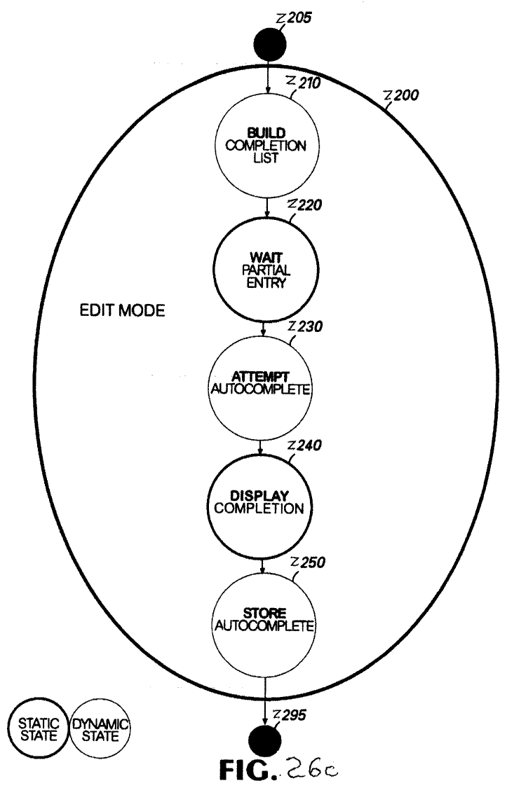

FIG. 26a illustrates an embodiment of a computer 10 suitable for supporting the operation of the preferred embodiment of the disclosure. Other configurations are included within the scope of this disclosure. As shown in FIG. 26a, the computer 10 may operate in a networked environment with logical connections to a remote computer 11. The logical connections between the computer 10 configured for implementation of this disclosure and the remote computer 11 are represented by a local area network 12 and a wide area network 13. Those of ordinary skill in the art will recognize that in this client/server configuration, the remote computer 11 may function as a file server or computer server.

The computer (CPU) 10 includes a processing unit (PU) 14. The computer may also include system memory 15 (including read only memory (ROM) 16 and random access memory (RAM) 17), which is connected to the PU 14 by a system bus 18. The preferred computer 10 utilizes a BIOS 19 (Basic Input/Output System), which is stored in ROM 16.

Those skilled in the art will recognize that the BIOS 19 is a set of basic routines that helps to transfer information between elements within the computer 10. Those skilled in the art will also appreciate that the present disclosure may be implemented on computers having other architectures, such as computers that do not use a BIOS. Additionally, the present disclosure is not limited to computers that utilize ROM or RAM for system memory. Other technologies such as electronically programmable ROM (EPROM), ultraviolet light erasable and electronically programmable ROM (UVEPROM), electronically erasable and programmable ROM (EEPROM), FLASH and bubble memory may also be used.

Within the computer 10, various devices may be connected to enhance the utility and performance of the computer. A local hard disk drive 20 may be connected to the system bus 18 via a hard disk drive interface 21. A floppy disk drive 22, which is used to read or write a floppy disk 23, may be connected to the system bus 18 via a floppy disk drive interface 24. A CD-ROM drive 25, which is used to read a CD-ROM disk 26, may be connected to the system bus 18 via a CD-ROM interface 27. A health care provider or medical coder enters commands and information into the computer 10 by using input devices such as a keyboard 28, and/or pointing devices such as a mouse 29. Typically, these input devices are connected to the system bus 18 via a serial port interface 430 or a parallel port interface (not shown in FIG. 4). Other types of pointing devices (not shown in FIG. 4) include track pads, track balls, pens, head trackers, data gloves and other devices suitable for positioning a cursor on a computer monitor or display 31. A monitor 31 or other kind of display device may be connected to the system bus 18 via a video adapter 32.

The computer may be connected to a network of other computers or devices. A remote computer 11 in a networked environment is connected to a remote memory storage device 33. This remote memory storage device 33 is typically a large capacity device such as a hard disk drive, CD-ROM drive, magneto-optical drive or the like. The personal computer 10 may be connected to the remote computer 11 by a network interface 34, which is used to communicate over the local area network 12.

The computer 10 may also be connected to the remote computer 11 by a modem 35, which is used to communicate over the wide area network 13, such as the Internet. The modem 35 is connected to the system bus 18 via the serial port interface 30. The modem 35 also can be connected to the public switched telephone network (PSTN) or community antenna television (CATV) network. Although illustrated in FIG. 26a as external to the computer 10, those of ordinary skill in the art will quickly recognize that the modem 35 may also be internal to the personal computer 11, thus communicating directly via the system bus 18. It is important to note that connection to a remote computer 11 via either the local area network 12 and the wide area network 13 is not required, but merely illustrates methods of providing a communication path between the computer 10 and the remote computer 11.

Although other internal components of the computer 10 are not shown, those of ordinary skill in the art will appreciate that such components and the interconnection between them are well known. Accordingly, additional details concerning the internal construction of the computer 10 need not be disclosed in connection with the present invention.

Those skilled in the art will understand that program modules such as an operating system 36, application programs 37a-N, and data are provided to the computer 10 via computer-readable or machine readable media. The computer-readable media may include the local or remote memory storage devices, which may include the local hard disk drive 20, floppy disk 23, CD-ROM 26, RAM 17, ROM 16, and the remote memory storage device 33. In the preferred computer 10, the local hard disk drive 20 is used to store data and programs, including the operating system and programs.

FIG. 26b is a simplified block diagram illustrating one embodiment of the interaction between the computer hardware 50, the preferred operating system 36, and an application program 37a. Referring now to both FIGS. 26a and 26b, when the computer 10 is turned on or reset, the PU 14 is forced to begin program execution at a specific memory location in the ROM 16. This specific memory location corresponds to the beginning of the bootstrap routine contained in the BIOS 19. The bootstrap routine functions to load the operating system 36 from the hard disk drive 20 into the RAM 17. Once the operating system 36 is loaded into RAM 17, the PU 14 executes the operating system 36 and causes the visual elements associated with the user interface of the operating system 36 to be displayed on the monitor 31.

The operating system 36, in conjunction with the BIOS 19 and associated device drivers, may provide the basic interface between the computer's resources, the user, and the application program 37a. The operating system 36 interprets and carries out instructions issued by the user and/or application program(s). For example, when the user wants to load an application program 37a, the operating system 36 interprets the instruction (e.g., double clicking on the application program's icon) and causes the PU 14 to load the program code into RAM 17 from either the local hard disk drive 20, floppy disk 23, CD-ROM 26, or the remote memory storage device 33. Once the application program 37a is loaded into the RAM 17, it is executed by the PU 14. For larger programs, the operating system 36 causes the PU 14 to load various portions of program, or program modules, into RAM 17 as needed. In addition, several applications programs (37a-N) can be loaded into RAM at the same time. In this scenario, the operating system 36 will switch the PU 14 execution time between applications based on user requests, application program request, or by a time-sliced allotment of the processing time of PU 14.

The operating system 36 may provide a variety of functions or services that allow an application program 37a to easily deal with various types of input/output (I/O). This allows the application program 37a to issue relatively simple function calls that cause the operating system 36 to perform the steps required to accomplish various tasks, such as displaying text on the monitor 31 (FIG. 26a) or printing text on an attached printer (not shown). Generally described (with reference to FIG. 26b), the application program 37a communicates with the operating system 36 by calling predefined functions provided by the operating system 36. The operating system 36 responds by providing the requested information in a message or by executing the requested task. In addition, the operating system may interface to the hardware components 50 in responding.

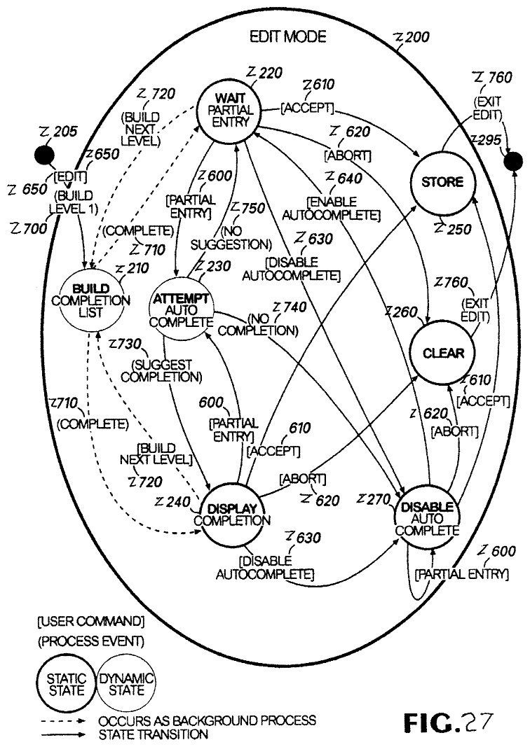

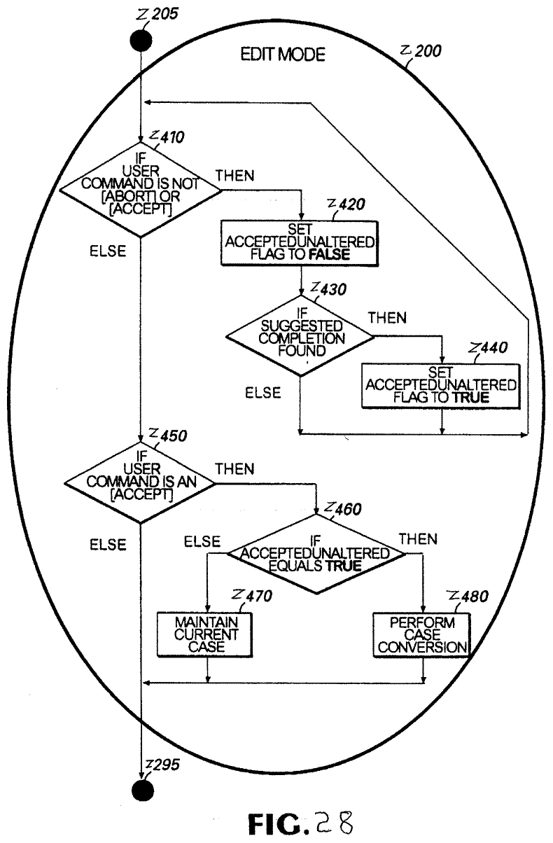

FIG. 26c may be used to illustrate the simple state flow for the autocomplete process including entering edit mode Z200 for an active cell (i.e., a space for entering a single alphanumeric character), entering a partial text or data item comprising one or more alphanumeric characters Z210, receiving a suggested completion Z240 (comprising one or more completed words), and accepting the suggested completion Z250, Z295. FIG. 27, which provides a full state diagram of the AutoComplete process, may be used to describe the AutoComplete process in detail as consisting of a state machine with user commands and process events inducing transitions between states. Finally FIGS. 28, 29a, 29b and 30 may be used to describe additional detail for several aspects of the AutoComplete process.

### AutoComplete User Interface

The AutoComplete user interface in one embodiment subject of the disclosure comprises eight functional areas: (1) Entering edit mode for an active cell; (2) Entering a partial data entry and finding a unique match; (3) Accepting a suggested completion; (4) Entering a partial data item over a suggested completion; (5) Entering a partial data entry and finding multiple matches; (6) Entering a partial data entry that disables further searches; (7) Obtaining a case conversion for a partial data entry; and (8) Entering a partial data entry where there are no associated cells. Each of these functional areas will be described with references being made to the user screens in FIGS. 3a-f and the state flow diagram in FIG. 26a.

Referring to FIG. 10, the health care provider may enter text in the description block or space 1002a regarding the patient. This may be a diagnosis or note pertaining to the examination or treatment. In one embodiment, text may be entered directly into the text block 1002a. The text may be subject to the autocorrect function (edit function) described below. The text may also be subjecting to the autofill of suggested code(s) entered into the code space 1001a. It will be appreciated that the suggested codes will be determined upon the health care provider's text entry into the text block 1002a. In another embodiment, the health care provider or coder may directly enter one or more codes into the code space 1001a.

Referring to FIGS. 10c and 27, in one embodiment, the edit mode for the text block may be manually activated entered at point by pressing a function key. In another embodiment, the edit function is automatically activated. The cursor may be located in the middle of text space 1002a, illustrating that text space 1002a is currently active with the edit mode being enabled. Referencing FIG. 27, upon entering edit mode Z205, a list of completed data items is generated in the BUILD Completion List state Z210. The list of completed data items may generated from stored completed data items pertaining to the text block 1002a of FIG. 10c or from a preloaded computer dictionary. The completed data items may be previously entered text entries. As will be described in more detail herein below, the list of completed data items will only contain one entry for each unique data item. It should be noted that the user may also enter the edit mode automatically by initiating a text entry in the text block. After generating the completed text data item list, (FIG. 10a containing text entry for sprain on left side of left knee 1002a) the alternate completed entries in code space 1001a may be displayed. Referencing FIG. 10c, if a partial text entry is entered, e.g., “Torn lig”, the screen will display a suggested completion text is reverse video of “ament”. Referencing FIGS. 26c and 27, when the partial text entry of “Torn lig” is entered, the WAIT Partial Entry state Z420 is entered.

### Entering a Partial Data Entry and Finding a Unique Match:

Entering a partial data or text entry will cause a transition to the ATTEMPT AutoComplete state Z230 and invoke a search of the list of completed data items to find a completed entry that uniquely matches the partial text entry. This can be completion of the text entry in FIG. 10c 1002a or matching code entries in 1001a. The DISPLAY Completion state Z240 is then entered and the suggested completion text is displayed in the text space 1002a and the suggested code(s) may be displayed in 1001a. A suggested completion can be presented to the user by displaying the portion of the suggested completion that contains the partial text or entry in normal video and the remainder of the suggested completion in reverse video e.g., having a distinguishing text color, text background or border. The reverse video is illustrated in FIG. 10a by initialization and brackets, e.g., [ ]. The suggested code can be similarly displayed.

Accepting a suggested completion. Referring to FIG. 10c, the health care provider has entered “torn lig” 1002a. The autocomplete functions displays the letter string “ament” immediately after the “g” of “torn lig”. At this point the user can either accept the suggested completion or enter an additional partial completion, i.e., by typing an additional character such as “g”. The scenario in which the user accepts the suggested completion is illustrated in FIG. 27 by entering the STORE AutoComplete state Z250. If the user accepts the suggested completion by pressing the enter key, the edit mode Z200 is exited at point Z295 (shown in FIG. 27). It will now be appreciated that the text states “torn ligament”. The suggested code may be similarly accepted. In another embodiment, acceptance of the suggested text, creating “Torn ligament” may result in additional suggested codes being displayed.

A suggest code may be entered into the code space 1001a. FIG. 10c illustrates the insertion of the ICD code for “repair, primary, torn ligament and/or capsule, knee, collateral”. The device may be programed to display both the ICD and CPT codes. Alternatively, the health care provider may select the specific code to be displayed. Note also that the code descriptor may also be displayed in 1001a. This will facilitate the health care provider electing whether to accept the code or, when multiple codes may be displayed, to select among the choices.

It will be appreciated that the EHR format of FIGS. 10a-10f is only one embodiment. Other configurations may be used and additional types of information may be contained within the display. See for example FIGS. 11 and 13.

### Entering a Partial Data Item Over a Suggested Completion:

Referring now to FIG. 10c, instead of accepting the suggested completion, the user may modify the partial data item by entering an additional letter character (e.g., “d”) into the partial word text entry “torn lig” within 1002a. This action may result in another search of the list of completed data items for a completed entry uniquely matching the modified partial data entry “ligd”. There will be no such entry. Accordingly, the closest matching word is “ligament”. Again, the completed data item “ligament” may be displayed in the text space 1002a next to the letter string “ligd”. The suggested autocorrect may be in reverse video (indicated in FIG. 10b by the [ ] brackets). If the user elects to again modify the partial data item by entering the additional character “m”, then the list of completed data items will be searched to identify a suggested completion for the twice modified partial data entry “ligam”. In this example, the completed term “ligament” may again be displayed in reverse video.

### Entering a Partial Data Entry and Finding Multiple Matches:

The results of entering a partial data entry may have multiple matching items. The partial entry “li” could trigger the suggested completion word text of “liver” or “ligament”. Both suggested entries may be shown in reverse video. In one embodiment, the suggested autocomplete or autocorrect entry may be shown beneath the subject letter string “li”.

Entering a Partial Data Entry that Disables Further Searches:

Referencing FIG. 10c, if the user enters the letter “i”, after “lig” the modified partial data entry of “ligi” will not have a matching completed data item in the completed data item list. When a partial text item is entered and there are no matching completed text items in the computer vocabulary (maintained in static memory) then the partial text entry is displayed in the text space. However, it will be appreciated that the disclosure includes an embodiment wherein the entry “ligi” will still trigger the word “ligament” (in reverse video) on the display as a suggested appropriate word text for a user error in spelling. This would be an application of an autocorrect application program. The application program may be enabled to suggest possible corrective word text.

Continuing with the partial text entry, if the user enters additional characters, the list of completed data items may not be searched. The reason for disabling further searches is that if the completed text item list does not contain a match for a partial text entry (i.e., “ligi”), then it will not contain a match for any partial data entry beginning with the previous partial data entry.

### Obtaining a Case Conversion for a Partial Data Entry:

The process of the case conversion in the AutoComplete process is illustrated. In FIG. 10e, the partial data entry “liga” has been entered and a suggested completion of “ligament” has been found and displayed 1002a. Alternatively, the portion of the suggested completion that matches the partial data entry may be displayed in normal video in conformance with the capitalization of the partial text entry “liga”. The suggested completed text entry containing the upper case “L” is displayed in reverse video. If the user accepts the suggested completion by pressing the enter key, the capitalization in the active cell will be adjusted to correspond to the capitalization of the completed data item.

### AutoComplete as a State Machine:

The detailed operation of AutoComplete can be described in the form of a state machine. In general, a state machine is a means to illustrate the operation of a process by identifying the various unique states in which the process can exist and identifying what events or circumstances will cause the process to transition between those states.

Referring to FIG. 27, a diagram of the AutoComplete state machine Z200 is shown as consisting of two types of states: static, depicted as a solid circle; and dynamic, depicted as a broken circle. A static state is a steady state in which an external event must occur in order to transition to a different state. In the absence of an external event, a process can remain in a static state indefinitely. A dynamic state is a momentary or transitory state in which a process only exists while performing a specific function or task. Upon completion of the task, a transition to another state will occur. A dynamic state generally comprises the execution of an algorithm and is exited upon completion of that algorithm.

The AutoComplete process comprises 3 static states and 4 dynamic states. These AutoComplete states include static states:
a. WAIT Partial Entry Z220;
   DISPLAY Completion Z240
   DISABLE AutoComplete Z270; and dynamic states:
   BUILD Completion List Z210;
   ATTEMPT AutoComplete Z230;
   STORE Z250; and
   CLEAR Z260.

Three types of transitions exist for the AutoComplete state machine and include: User commands; Process events; and Background events. The User commands, indicated by [xxxxx], occur in response to actions taken by the user. The Process events, indicated by (xxxxx), occur automatically upon the completion of processing in a dynamic state. The Background events, indicated by (xxxxx) and drawn with dashed lines, occur during the idle time between user commands.

The AutoComplete user commands include:
a. [Partial Entry] Z600: Partial data item for a cell;
   [Accept] Z610: Accepts a suggested completion;
   [Abort] Z620: Rejects the data in the active cell;
   [Disable AutoComplete] Z630;
   [Enable AutoComplete] Z640; and
   [Edit] Z650: Enables the edit mode for a cell.

The AutoComplete process events include:
a. (Suggested Completion) Z730;
   (No Completion) Z740;
   (No Suggestion) Z750; and
   (Exit Edit) Z760.

The AutoComplete background process events include:
a. (Build Level 1) Z700;
   (Complete) Z710; and
   (Build Next Level) Z720

Upon entering the edit mode for an active cell, the AutoComplete process will initiate the performance of two functions. The first function is the generation of the list of completed text or code items associated with the alphanumeric text entries 1002a. In the preferred embodiment, the completed data item list will be generated in a tiered approach as a background process. The tiered approach consists of building the list of completed data items one level at a time where each level includes a specific number of possible alphanumeric character spaces. The second function is to identify and provide a suggested completion for a partial text entry.

Referencing FIG. 27, entering the [Edit] user command Z650 causes the edit mode Z200 to be entered as shown at point Z205. The (Build Level 1) process command Z700 is issued automatically upon entering edit mode and the BUILD Completion List state Z210 is entered. In the BUILD Completion List state Z210, the first level of the completed data item list is generated. In the preferred embodiment, the first level includes the 50 alphanumeric character spaces that are most closely associated with the active cell. As will be described later, this includes the associated character spaces that are physically closest to the active space. If there are a limited number of text entries (i.e., less than 50 alphanumeric character spaces), the first level of the completion list will include all of the associated entries using a pre-entered dictionary, past usage or compatible code descriptors. After the first level of the completion list has been generated, a (Complete) process event Z710 is executed and a transition to the WAIT Partial Entry state Z220 occurs. In the WAIT Partial Entry state Z220, the application program waits for the reception of a user command, e.g., acceptance or entry of another alphanumeric character.

If there is idle time between entering user commands in the WAIT Partial Entry state Z220, and the completion list has not been fully generated, a (Build Next Level) background process event Z720 will be issued resulting in a transition to the BUILD Completion List dynamic state Z210. (Note, this event may also be issued in the DISPLAY Completion state Z240.) When the next level of the completion list has been generated, a (Complete) process event Z710 will be issued and a transition to the WAIT Partial Entry state Z220 will occur (or a transition to the previous state). The (Build Next Level) background process event Z720 will continue to be issued until the list of completed data items has been fully generated.

In the WAIT Partial Entry state Z220, four user commands are accepted: [Partial Entry], [Disable AutoComplete], [Abort], and [Accept]. If the user provides [Partial Entry] Z600, a transition to the ATTEMPT AutoComplete state Z230 will occur. This transition will occur in response to entering the first character in the text space. This is an improvement over similar implementations, which require the entry of several characters prior to attempting to automatically complete the entry.

In the ATTEMPT AutoComplete state Z230, the partial text entry will then be used as a character mask to search through the list of completed text items for a unique match. A unique match exists if there is only one item in the completed text item list that begins with the same character or characters defining the partial data entry. A unique match is further defined as, given that an N character partial entry has been entered, if there is one and only one text item in the completed text item list that begins with these N characters, then that data item is a unique match. If more than one entry matches the first N characters, then a unique match does not exist.

The ATTEMPT AutoComplete state Z230 can be entered prior to fully generating the list of completed text items. If this occurs, the search for a suggested completion will be limited to the completed text item list in its current state (i.e., the levels that have been generated). But, if updating the list of completed text items results in destroying the unique status of the partial text entry, the current AutoComplete suggestion remains intact until either the user accepts the text item or enters a subsequent partial entry.

As a result of entering the ATTEMPT AutoComplete state Z230, one of three possible outcomes will be realized: (1) a unique match will be found; (2) no matches will be found; or (3) multiple matches will be found.

If a unique match is found, then a (Suggested Completion) process event Z730 is issued, which results in a transition to the DISPLAY Completion state Z540. Here, [Partial Entry] Z600 containing the partial data entry “liga” was entered in the WAIT Partial Entry state Z220 and this resulted in a transition to the ATTEMPT AutoComplete state Z230. Upon finding the unique match “ligament”, the (Suggested Completion) process event Z730 issued, which resulted in a transition to the DISPLAY Completion state Z240 for displaying the suggested completion in the text space or code space.

If no matches are found, the (No Completion) process event Z740 is issued, which results in a transition to the DISABLE AutoComplete state Z270. Examples of this scenario include the partial data entry “lj” that has no matches.

If multiple matches are found, the (No Suggestion) process event Z750 is issued, which results in a transition back to the WAIT Partial Entry state Z220. An example of this transition and the resulting display occurs where a partial data entry “I” matches both the completed text items “liver” and “ligament”.

The [Partial Entry] command Z600, entered in the WAIT Partial Entry state Z520, can also consist of multiple characters. FIG. 10d illustrates that if the partial data entry is “L”, the search of the completed data item list may result in finding the suggested completion “ligament” and “lumen”. If entering additional characters to distinguish a partial data entry from multiple matches, i.e., the character “i” can be appended to partial data entry “I” in FIG. 10d to form partial data entry “li” and resulting eliminating “lumen” as a possible entry. This results in the from the ATTEMPT AutoComplete state Z230. A (Suggested Completion) process event Z730 is then issued and a transition to the DISPLAY Completion state Z540 occurs with the display of FIG. 3d being produced. It will be appreciated that the code space 1001a may also be populated with suggested codes and descriptors.

In contrast, if the character “o” is appended to the partial text entry “li”, partial text entry “lio” will be formed and a transition to the ATTEMPT AutoComplete state Z230 will occur. The search of the completed text item list will result in finding no matches for partial text entry “lio”. The (No Completion) process event Z740 will be issued and a transition to the DISABLE AutoComplete state Z270 will occur.

In the WAIT Partial Entry state Z220, the [Disable AutoComplete] command Z630 can also be entered. In an embodiment, the [Disable AutoComplete] command Z630 may consist of moving the insertion point or cursor in the edit window. Other methods could also be utilized such as pressing a function key. Entering this command causes a transition to the DISABLE AutoComplete state Z270.

The final two commands that can be entered in the WAIT Partial Entry state Z220 are [Abort] Z620 and [Accept] Z610. Entering the [Abort] Z620 command results in a transition to the CLEAR AutoComplete state Z260, in which the pre-edited contents of the alphanumeric character space will be restored. The (Exit Edit) process event Z760 will then be issued and the edit mode Z200 will be exited at point Z295. Any partial data entries displayed prior to the [Abort] Z620 command will be erased. Entering the [Accept] command Z610 causes a transition to the STORE AutoComplete state Z250 in which state the contents displayed in the alphanumeric character space are stored and the (Exit Edit) process event is issued to exit the edit mode Z200 at point Z295. In the preferred embodiment, an [Accept] Z610 can be issued by entering a carriage return or by using the key pad or mouse to change active cells. An [Abort] Z620 can be issued by entering the [ESC] key. Other methods could also be used to implement these commands and are contemplated by the present invention.

The DISPLAY completion state Z240, operates to display a suggested completion in the alphanumeric character space. If the completion list is not fully generated, the (Build Next Level) background event Z720 may be issued as described above. While in the DISPLAY completion state Z240, the user can enter the [Partial Entry] Z600, [Accept]Z610, [Abort] Z620 or [Disable AutoComplete] Z630 commands.

Entering [Partial Entry] Z600 in the DISPLAY Completion state Z240 causes a transition to the ATTEMPT AutoComplete state Z530. The completed data item list will then be examined for a unique match of the partial entry. As a result of entering the ATTEMPT AutoComplete state Z530 from the DISPLAY Completion state Z540, two possible outcomes can be realized: (1) a unique match will be found or (2) no matches will be found.

Entering [Accept] Z610 in the DISPLAY Completion state Z540 causes a transition to the STORE AutoComplete state Z250. Entering this state from the DISPLAY Completion state Z240 will invoke a case conversion if one is required. Entering [Abort]Z610 in the DISPLAY Completion state Z540 will have the same response as entering it in the WAIT Partial Entry state Z520 discussed above.

Entering the [Disable AutoComplete] command Z630 in the DISPLAY completion state Z240 will cause a transition to DISABLE AutoComplete state Z270. The contents of the alphanumeric character space will be maintained; however, the portion of the text entry that had been displayed in reverse video will be changed to normal video to indicate that AutoComplete is disabled.

The DISABLE AutoComplete state Z270 operates to eliminate the overhead of searching through the completed data item list when it is known that a match will not be found. For example, there are no matching entries in the completed data item list for partial data entry “Lio”. Appending additional characters to the partial data entry “Lio” will not change this situation. Thus, the DISABLE AutoComplete state Z270 will allow the user to continue entering partial entries without burdening the processing unit Z414 of FIG. 26a with fruitless searches.

The DISABLE AutoComplete state Z470 may also be entered if the completed text item list is empty. This situation will occur when the first text item in a new area is being entered or all other text items have been filtered out. In an embodiment which filters out numeric data items, editing a cell within a column of numbers will result in issuance of a (No Completion) Z740 process event in the ATTEMPT AutoComplete state Z230 and a subsequent transition to the DISABLE AutoComplete state Z270.

In the DISABLE AutoComplete state 270, an [Abort] Z620, [Accept] Z610, or [Enable AutoComplete] Z640 commands can be entered. The responses to the [Abort]Z620 and [Accept] Z610 commands are identical to the responses generated in the DISPLAY Completion state Z240. Entering an [Enable AutoComplete] Z640 command will cause a transition to the WAIT Partial Entry state Z220. In the preferred embodiment, the [Enable AutoComplete] Z640 command can take the form of back-spacing over or deleting the last entered alphanumeric character(s) that caused the (No Completion) Z240 process event to be issued or returning the insertion point to the end of the partial data entry. As an example, in the situation of a partial entry “lio” when the DISABLE AutoComplete state Z270 is active. Backspacing over the letter “o” will cause the issuance of the [Enable AutoComplete] Z640 command and a transition to the WAIT Partial Entry state Z220; however, the ATTEMPT AutoComplete state may optionally not be entered until a new partial text item is entered.

The CLEAR AutoComplete state Z260 can be entered from the WAIT Partial Entry state, the DISPLAY Completion state Z240, or the DISABLE AutoComplete state Z270 upon the execution of the [Abort] Z620 command. The [Abort] Z620 command operates to erase the text that is currently being displayed in the alphanumeric character space and then exiting the edit mode Z200. In addition, the contents of the active cell prior to entering the edit mode Z200 will be restored.

In the STORE AutoComplete state Z250, the data being displayed in the alphanumeric character space is stored and the (Exit Edit) process event Z760 is executed to exit the edit mode Z200 at point Z295. This process occurs independent of the prior state; however, when entering from the DISPLAY AutoComplete state Z240, a case conversion may be performed on the suggested completion.

FIG. 10f illustrates the entry (or selection) of the term “torn ligament”. The entry causes a search of the codes for compatible code and description entries. FIG. 10f illustrates the insertion of suggested multiple CPT codes and descriptors. The health care provider may select one or more of the suggested entries.

It will be appreciated that the display of the code descriptors will facilitate the health care provider entry of a sufficiently clear and complete description of the diagnosis or treatment. The descriptors need not be stored in the EHR code space but rather presented only during the drafting of the EHR. Stated differently, the descriptors need not be stored as part of the text of the EHR. Further, the descriptors appearing in the code space during drafting or entry of the patient diagnosis, etc., can prompt the clinician to provide additional detail. For example, if the clinician does not provide whether the injured knee is the right or left, the displayed descriptor will state the knee is unspecified. Similarly if the health care provider does not specify the ligament is injured, the descriptor will state “ligament unspecified”, thus prompting the clinician to provide more specific information if possible. The correct or consistent use of descriptors, as suggested by the repeated display of code descriptors, will facilitate correct or uniform terms to describe a patient condition or diagnosis by clinicians. The clinician may select one of the suggested descriptor, thereby selecting the appropriate code for invoicing and EHR indexing. The use of uniform terms will facilitate the understanding of a subsequent clinician reviewing the EHR or portion of the EHR provided in response to an information request. The enhanced uniform usage of terms will also facilitate the use of common terms in searching the patient's medical history. This may also cause the code descriptors, established by various organizations, to be subject of editing and revision suggestions from the clinician. This will clarify the distinctions between the various codes and thereby reduce erroneous entries being created. This will facilitate both the billing function of the codes and the use of the codes for indexing of EHR and as a search tool. The codes indexing a patient's EHR will allow for smart searching. It will not be necessary for the current clinician to have to guess what medical terms have been used by a prior clinician to describe the patient's condition of treatment.

In a synergistic fashion, the autocorrect and autocomplete functions discussed in this disclosure will improve the descriptions entered by the clinician to improve the accuracy of the entered codes. In corresponding fashion, the code descriptors will suggest alternate and distinctive entries that may be used by the clinician to describe the diagnosis and/or treatment procedures. This can result in more robust and meaningful records that will be available to subsequent clinicians. Stated differently, there will not be two separate languages used for recording a patient's health records, i.e., a language used for determining correct billing and a language used by clinicians to describe the patient's condition (leading to diagnosis) or treatment.

Note further that the use of the codes as a search tool for a clinician to promptly search and receive information of the patient's medical history does not require that a central clearing house or similar entity be created that coordinates search requests for patient's prior medical history. The search request, specifying the patient by common identity markers (name, date of birth, SSN, etc.) can be simultaneously submitted electronically to multiple clinicians. It will be appreciated that these requests will contain evidence of patient's consent for disclosure of records.

Further, requests may be submitted through health insurance companies or other third party payors to be forwarded to other clinicians that the insurance company records disclose have received insurance reimbursement for treatment of the identified patient and utilizing the same or similar codes.

### Case Conversion

The case conversion algorithm is a unique aspect of the AutoComplete process and gives it the ability to handle data items with mixed upper and lower case characters. In general, the AutoComplete algorithm is case insensitive during the data entry process. But, when a suggested completion has been accepted, the AutoComplete process will adjust the capitalization of the data item to be consistent with matching entries. The purpose behind the case conversion is to provide consistency for the text items in the EHR. If a text item is being entered in lower case, and a matching completed text item is found that is in upper case, then the matching item will be displayed as a suggested completion. The portion of the suggested completion that matches the partial text item (ignoring the capitalization) will be shown in normal video on the display screen and in the capitalization that the partial text item was entered. The portion of the suggested completion that does not include the partial text item will be displayed in reverse video and in the capitalization that corresponds with the suggested completion. Accepting the suggested completion will result in adjusting the case of the partial entry to be the same as that of the suggested completion. But, if the user changes the insertion point or in some other way executes a DISABLE AutoComplete command Z630, then accepting the text in the alphanumeric character space will result in maintaining the case of the characters as displayed in the alphanumeric character space.

Turning to FIG. 28, edit mode entrance point Z205 indicates that the case conversion algorithm becomes active when the edit mode Z200 has been entered for an alphanumeric character space. The process blocks shown in FIG. 6 are executed during various states of the AutoComplete process. Overall, the case conversion algorithm maintains an “Accepted/Unaltered” flag to indicate whether or not a case conversion should be performed upon exiting the edit mode Z200. When the Accepted/Unaltered flag is equated to FALSE, as in process block Z420, then the case conversion will not be performed upon exiting the edit mode Z200. Conversely, a value of TRUE in the Accepted/Unaltered flag will cause a case conversion to be performed. Thus, the Accepted/Unaltered flag is examined upon exiting the edit mode Z200, to determine if a case conversion should be performed. Decision block Z410 illustrates that for each user command received, other than an [Abort] Z420 or [Accept] Z450, the THEN branch is followed. For each user command invoking the THEN branch of process block Z410, the AcceptedUnaltered flag is set to FALSE as shown in process block Z420. If the user command results in producing a suggested completion (i.e., for [Partial Entry] Z200 commands with unique matches), the THEN branch of decision block Z430 will be followed and process block Z440 will set the Accepted/Unaltered flag to TRUE. If the user command does not result in finding a suggested completion, then the ELSE branch of decision block Z430 is followed. In either of these cases, processing will return to decision block Z410 and the next user command will be processed.

Once the [Abort] Z620 or [Accept] Z610 commands are received, the ELSE branch of decision block Z410 will be followed and decision block Z450 will be entered prior to exiting the edit mode. If the received user command was an [Accept] Z450 command, then the Accepted/Unaltered flag will be examined in decision block Z460 to determine if a case conversion should be performed. If the Accepted/Unaltered flag is equated to TRUE, process block Z480 will be entered to perform the case conversion; however, if the Accepted/Unaltered flag is equated to FALSE, then the current case displayed in the active cell will be maintained. In either case, the edit mode for the active cell will be exited at point Z295. In summary, whenever a partial entry results in producing a suggested completion, a case conversion will occur upon a subsequent [Accept] Z410 command. If the user enters any command other than [Accept] Z410, then the case conversion will be disabled until a subsequent suggested completion is produced.

Similar to the above described autocomplete and autocorrect functions, the disclosure also teaches that text entries with the diagnosis/treatment/test space 1002a may trigger a suggested entry into the code space. This step requires that there be a listing or vocabulary of code entries correlated with the text entry. Accordingly, entry of “torn ligament” in space 1002a of FIG. 10f, will prompt the entry of code numbers/letters related to the text “torn ligament” 1001a.

In an alternate embodiment, the entry of text in diagnosis/treatment/test space 1002a of “torn ligament” will trigger a display of a suggested diagnosis, etc., with the corresponding code. In this variation, it will be appreciated that the diagnosis text or name entered into the code space 1001a is correlated with the diagnosis description appearing in 1002a. This embodiment has a vocabulary of diagnosis/treatment/test descriptors correlated with the possible text descriptions entered by the health care provider. The code appears with the descriptor. Stated differently, the alpha-numeric codes are correlated with possible text descriptions that may be entered by the health care provider.

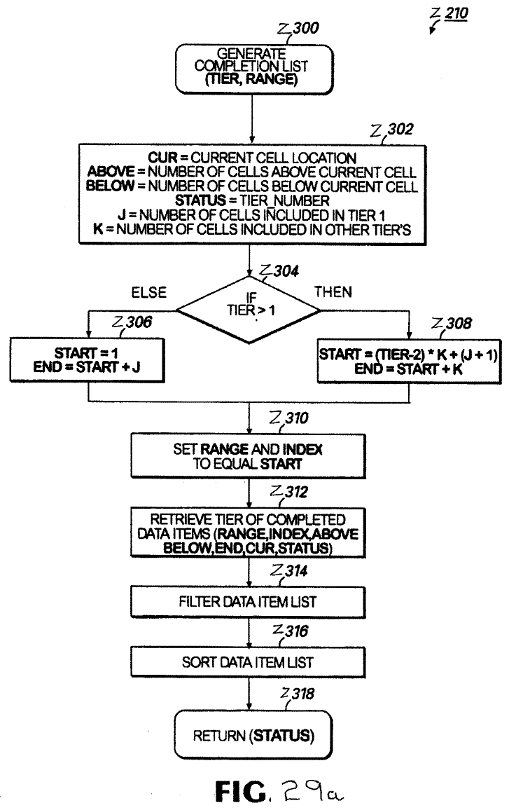

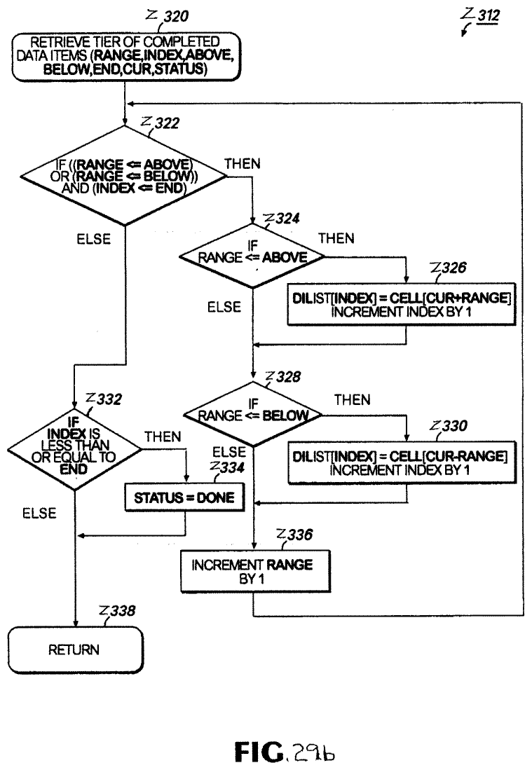

Turning now to FIG. 29a, the detailed operation Z210 of generating the completed text item list Z300 is shown. The completed text item list used in the AutoComplete process is a dynamic list (i.e., is built on the fly for each data entry attempt). In contrast to a static list, a dynamic list does not require permanent memory resources. Therefore, once a text item has been entered and accepted by the user, the memory occupied by the completed text item list can be released and reallocated to other resources. The trade off in using a dynamic list rather than a static list is the processing overhead required to generate a unique list upon each entrance of the edit mode. In one embodiment, this processing overhead is minimized by utilizing a tiered generation technique. This tiered technique includes building a first text item list of entries most closely associated with the alphanumeric character space (current or active cell) and, during idle time between user commands, augmenting the list with other associated text items. This process will continue until the entire list has been completed. Thus, in this tiered technique, priority is given to user input, and text entry is not delayed or “shuttered” while the text item list is being generated.

The first aspect in building the text item list is determining the particular text items that are associated with the alphanumeric character space. The preferred embodiment utilizes a table determination algorithm to define the borders of an EHR. The algorithm defines a table as a set of text items surrounded by “white space” or empty cells, i.e. a portion of the text space 1002a that may contain a single alphanumeric character or not, i.e., an empty cell. Ignoring certain exceptions such as text boxes, table headings, picture objects, and print area definitions, this algorithm defines a rectangular border that encompasses adjacent data items in the vertical horizontal and diagonal directions.

The process of determining the data items that are associated with an active cell (the portion of the text or code space that can accept a single alphanumeric character) can be accomplished in several ways. In the most liberal approach, all of the data items entered into a spreadsheet and any associated sheets could be considered to be associated with the active cell, and thus, become input into the data item list generation process. Although for some applications this may be a viable approach, the typical spreadsheet designer arranges associated data into columns. Hence, in the more restrictive approach, only data items in the same column and same table (using the term table as defined by the above algorithm) would be considered to be associated with the active cell. A further limitation would be to only include the block of data items that are in the same column as the active cell, encompass the active cell, and are bordered by white space, i.e., a portion of the text or code space that cannot accept an alphanumeric character.

The second aspect in building the text item list involves applying filters to the list of associated data items. Several filtering mechanisms can be employed. A first filtering mechanism includes limiting the text items to alphabetical or alpha-numeric entries, and hence, excluding numeric entries. A second filtering mechanism is the elimination of duplicate data items from the text item list. Thus, if a text item has been entered in a column multiple times, sorting through the text item list will not be burdened by examining redundant text items. Other filtering mechanisms can include (a) limiting the text items to include only those items that have been entered more than once; (b) limiting the text items to only include data that conforms to certain formatting restrictions; and (c) limiting the text items to entries that exceed a certain number of characters. In the preferred embodiment, the filtering mechanism is limited to the elimination of duplicate text items.

FIGS. 29a-b illustrate a possible implementation of the algorithm to generate a completion list. This algorithm is executed in the BUILD Completion List state Z300 shown in FIG. 29a. Entry block Z205 of FIG. 27 indicates that the generate completion list algorithm is invoked with input parameters TIER and RANGE. The TIER parameter is used to identify which level of the completion list is to be generated. The purpose of RANGE will be described later. In exit block Z318 of FIG. 29a, the generate completion list algorithm returns the parameter STATUS. STATUS indicates whether the completed data item list has been fully generated (i.e., STATUS is equated to DONE) or if subsequent levels need to be generated (i.e., STATUS is equated to TIER). This algorithm will be invoked as many times as necessary until a completion list is generated that comprises all of the text items that are associated with the alphanumeric character space.

Continuing with FIG. 29a, in process block Z302, the algorithm variables and parameters are initialized. The variable CUR is defined as the location of the alphanumeric character space. The variables ABOVE and BELOW define the boundaries of the alphanumeric character spaces associated with the active alphanumeric character space. These variables are equated to the number of associated alphanumeric character spaces above and below the current alphanumeric character space respectively. The variable J identifies the number of alphanumeric character spaces that are to be included in the first tier or level 1 generation of the completion list. The variable K defines the number of alphanumeric character spaces included in subsequent tiers of the completion list. In the preferred embodiment the values of J and K are set to 50 and 20 respectively; however, other values for these variables can be selected and are contemplated by the present invention.

In decision block Z304, the input parameter TIER is examined to determine which level of the completion list is being generated. If level 1 is being generated (TIER=1) then execution continues in process block Z306. In this block two additional parameters are initialized. The START parameter is used to identify the location of the next associated alphanumeric character space to be included in the completion list. The parameter END is used to define the location of the last associated alphanumeric character space to be included in the completion list for the level being generated. In process block Z306, these parameters are initialized for the first level. Thus, in the preferred embodiment the first level will include associated alphanumeric character spaces 1 to J, or the first 50 associated alphanumeric character spaces. If the TIER parameter is greater than 1 in decision block Z304, then processing continues in block Z308. If level 2 is being generated (TIER=2), process block Z308 equates START to J plus 1 (51 in the preferred embodiment) and END is equated to START plus K. Thus, for level 2, the START and END variables are set up so as to examine the next K alphanumeric character spaces associated with the active alphanumeric character space (spaces 51-70 in the preferred embodiment). For TIER N, space (N−2)*20+51 and the following K−1 spaces will be added to the completed text item list. Upon completion of process block Z306 or Z308 process block Z310 will be entered.

Process block Z310 initializes the INDEX variable by equating it to the value of START. INDEX is used as a counter to indicate when all of the spaces for the current level have been read and is also used as a pointer in the list of completed text items to identify the location to store the next text item. Input parameter RANGE is used as a pointer to indicate the distance above and below the active character space where the next text item is to be retrieved. When entering edit mode for the active character, the RANGE variable is equated to 1. The text character spaces immediately above and below the active space are at RANGE 1. The next two spaces above and below the active space are at RANGE 2. Thus, as text items are retrieved from associated spaces, the RANGE variable is incremented. The value of RANGE must be retained between subsequent calls to the BUILD Completion List algorithm.

Process block Z312 invokes the Retrieve Tier of Completed Text Items routine shown in FIG. 29b. This routine utilizes the variables RANGE, INDEX, ABOVE, BELOW, END, CUR, and STATUS in retrieving and building the next level of the completion list. Process block Z314 applies the appropriate filters to the completed text item list. Process block Z316 will sort the new filtered completed text item list in alphabetical order. Finally, exit block Z318 returns the STATUS variable to the calling routine.

Turning to FIG. 29b, the details of process block Z312 in FIG. 29a are provided. Entrance block Z320 in FIG. 29b indicates the starting point of the routine. In process block Z322 a check is performed to determine if all of the text items for the level being generated have been retrieved. This is accomplished by verifying that RANGE is less than or equal to ABOVE or BELOW and that INDEX is less than or equal to END. The program variables for level 1 will be initialized as follows:

a. START=1

END=50

ABOVE=1 (includes text character space Z328)

BELOW=1 (and comprises text character space Z326)

RANGE=1

INDEX=1

Continuing with the example, decision block Z322 in FIG. 29b determines that RANGE (equated to 1) is equal to ABOVE and less than BELOW, and that INDEX is less than END. Therefore, the THEN branch of decision block Z322 will be followed and decision block Z324 will be entered. In the THEN branch of decision block Z322, RANGE is examined in comparison to ABOVE and BELOW to determine which associated text character spaces should be selected. The basic premises of the algorithm is to select the next closest space to the active alphanumeric character space, giving preference to spaces above the active space. For instance, when building a level of associated text items, if there are spaces above and below the active text character space, they are selected by alternating between the space above and the space below the active alphanumeric character space. In some instances, there will be character spaces above the active alphanumeric character space but no text character spaces below the active alphanumeric character space or vice versa. In this case, only the character spaces above or below the active text character space are selected. Thus, if the bottom text character space in a column is being edited, then the J text character spaces above the active alphanumeric character space will be selected in the first level. Alternatively, if the text character space is being edited, then the J text character spaces below the active alphanumeric character space will be selected.

In process block Z324, if RANGE is less than or equal to ABOVE, then there are associated text character spaces above the active alphanumeric character space. Execution then continues in process block Z326 where the contents of the text character space at the location of the active alphanumeric character space plus RANGE is loaded into the completed text item list at the location of INDEX. In addition, INDEX is incremented by 1. Indecision block Z324, if RANGE is greater than ABOVE, then there are no associated text character spaces above the active alphanumeric character space and the ELSE branch will be followed. Whether or not process block Z326 is executed, processing will continue at decision block Z328. In decision block Z328, if RANGE is less than or equal to BELOW, then there are associated text character spaces below the active alphanumeric character space. Execution then continues in process block Z330 where the contents of the text character space at the location of the active alphanumeric character space minus RANGE is loaded in to the completed text item list at the location of INDEX. In addition, INDEX is incremented by 1. In decision block Z328, if the value of RANGE is found to be greater than BELOW, then there is no associated text character space below the active cell and the ELSE branch will be followed. Whether or not process block Z330 is executed, processing will continue in process block Z336. In process block Z336, RANGE is incremented by 1 and processing returns to decision block Z322.

Therefore, the overall effect of executing the THEN branch of decision block Z322 is to obtain the next two completed text items associated with the active letter string. In some instances, only one text data item will be retrieved. This will be the case when there are no associated text character spaces either above or below the active alphanumeric character space text string.

For example, after the first execution of the THEN branch of decision block 322, the variables will be set to the following values:

a. START=1

END=50

ABOVE=1

BELOW=11

RANGE=2

INDEX=3

In decision block Z322 for the current example, RANGE is no longer less than or equal to ABOVE. But, RANGE is less than BELOW and INDEX is less than END. Therefore, processing will continue to execute through the THEN branch of decision block Z322 and returning to process block Z322 until either (1) RANGE is greater than both ABOVE and BELOW, or (2) INDEX is greater than END. In the first case, the list of completed text items is fully generated. Thus, in decision block Z332, INDEX will be less than END causing process block Z334 to be executed, equating STATUS to DONE. In the second case, additional levels must be generated. Thus, in decision block Z332, INDEX will be greater than END resulting in returning STATUS equated to the value of TIER or the level that was generated.

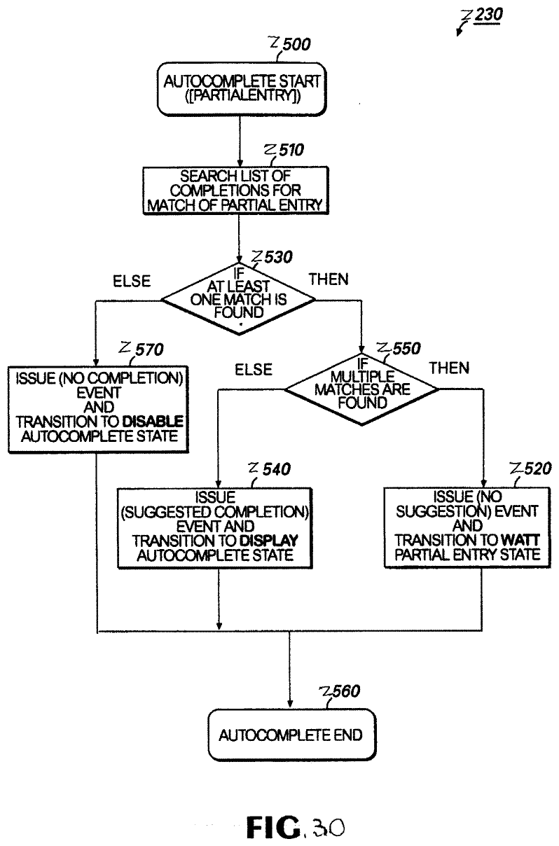

Referring now to FIG. 30, the AutoComplete algorithm invoked in the ATTEMPT AutoComplete state Z230 (shown in FIG. 27) is illustrated as a flow diagram. The ATTEMPT AutoComplete state Z530 is entered from the WAIT Partial Entry state Z520 or the DISPLAY Completion state Z540 upon providing a partial data entry.

Entrance block Z500 in FIG. 30 illustrates that the AutoComplete algorithm uses input parameter [Partial Entry] Z520. In process block Z210, the completed data item list is searched for items that match [Partial Entry]. The searching process can be accomplished using a binary search, sequential search, or various other searching techniques. If at least one match is found, the THEN branch of decision block Z530 will be followed and decision block 550 will be entered. At decision block Z550, if more than one match has been identified in the completed text item list, the THEN branch of decision block Z550 will be followed and process block Z520 will be entered. In block Z520 the (No Suggestion) process event Z750 will be issued, and a transition to the WAIT partial entry state Z520 will occur. Returning to decision block Z550, if only one match was identified in the completed data item list, the ELSE branch of decision block Z550 will be followed and process block Z540 will be entered. In process block 540, the (Suggested Completion) process event Z730 will be executed and a transition to the display completion state Z540 will occur.

Returning to decision block Z530, if no matches are found in the completed data item list, the ELSE branch of decision block Z530 will be followed and process block Z570 will be entered. In process block Z570, a (No Completion) process command Z740 will be executed and a transition to DISABLE AutoComplete state Z270 will occur. Block Z560 in FIG. 8 will then be entered and the AutoComplete algorithm will be exited.

From the foregoing description, it will be appreciated that the present disclosure provides a method to improve the efficiency and reliability of text entry in a EHR record by providing the ability for an automatic completion process utilizing a list of completed text items comprised of text associated with the medical diagnosis, treatment or test item being entered into the EHR. The disclosure also creates improved efficiency and reliability in the diagnosis, treatment and test codes associated with the text entry of the health care provider.

The foregoing method of the present disclosure may be conveniently implemented in one or more program modules. No particular programming language has been indicated for carrying out the various tasks described above because it is considered that the operation, steps, and procedures described in the specification and illustrated in the accompanying drawings are sufficiently disclosed to permit one of ordinary skill in the art to practice the instant invention. Moreover, in view of the many different types of computers and program modules that can be used to practice the instant invention, it is not practical to provide a representative example of a computer program that would be applicable to these many different systems. Each user of a particular computer would be aware of the language and tools which are more useful for that user's needs and purposes to implement the instant invention.

The above described features of the present disclosure have been described in relation to particular embodiments which are intended in all respects to be illustrative rather than restrictive. Those skilled in the art will understand that the principles of the present disclosure may be applied to, and embodied in, various program modules for execution on differing types of computers regardless of database application.

Alternative embodiments will become apparent to those skilled in the art to which the present disclosure pertains without departing from its spirit and scope. Accordingly, the scope of the present disclosure is described by the appended claims and supported by the foregoing description.

The population of the code space with one or more suggested codes is achieved by creating a data base of the codes and each code's descriptor. When the descriptor is entered in FIG. 10a into the text space 1002a, the corresponding code is entered in code space 1001a.

As described above, this disclosure teaches the health care provider entering diagnostic notes etc., electronically into a device. The disclosure facilitates this entry of data by auto-correct features, etc. The disclosure also teaches display of suggested codes based upon the text of the entered data. This step may include matching of entered text data with the text of code descriptors. However the disclosure includes direct matching of alphanumeric codes to the healthcare provider's entered text. This disclosure teaches the following steps as one embodiment of populating the code space with suggested codes and the clinician enters the description of the diagnosis or treatment, etc. First, the correlation of the data entry with all possible applicable codes. In a simple embodiment, the code space would become populated with all codes containing “knee” in the descriptor. As the clinician adds additional text, e.g., “torn ligament”, the codes pertaining to “sprains or tearing of knee ligament” would be substituted into the code space. In other embodiments, the program could enter all codes that were classified and correlated as applicable to torn knee ligaments. This could be further expanded to applications utilizing machine learning algorithms.

Such an algorithm would create a dynamic completion list, in contrast to solely utilizing pre-defined values. The completion list is generated from a set of data within the database that is associated with the data item being entered, and reflects the status of the database at the time the data item is being entered. The benefits associated with this aspect of the invention include: (i) providing a completed text item list that is automatically updated to reflect the current contents of the database; (ii) providing a completed text item list that is not encumbered by extraneous data entries that have no relationship with the item being entered; and (iii) providing an automatic completion feature that is not restricted to the use of a limited list of possible completions (i.e., a predefined data set).

A unique aspect of the generation process includes defining which entry text items or descriptions entered into the text space (diagnosis, treatment, test) 1002a are associated with the code being suggested 1001a. In the context of a generic database, the present invention provides several methods to perform this process. Generally, text entry items, e.g. diagnosis or procedure descriptions, that fall within the same category or a similar category to the code item being suggested are considered to be associated text items. Therefore, an advantage of the present disclosure is the ability to define which alphanumeric entries within a code database are related to a text item being entered by the clinician, to generate a list of suggested codes based on these associated diagnosis or treatment data entries, and to provide a dynamic completion code list which tracks the actual contents of the diagnosis or treatment, etc.

Electronic health records (EHR) track patient health care information over time. Further, the EHR records can easily identify patients due for preventive screenings or checkups. Also, the records can check the patient's health conforming to certain parameters such as blood pressure readings or vaccinations. Overall, the EHR can serve to monitor and improve overall quality of patient care. EHRs may focus on the total health of the patient, i.e., going beyond standard clinical data collection of care within a single health care provider's office, etc. EHRs are designed to share information with other clinicians, such as laboratories and specialists, so that the EHRs contain information from all the clinicians involved in the patient's care. An electronic health record (EHR) is created, managed, and consulted by authorized clinicians and staff across more than one healthcare organization. The EHR is formatted to be used both intramurally (within the “walls” of a single clinician similar to an electronic medical record (EMR) and to be used inter-mural fashion, i.e., shared and accessible to multiple authorized clinicians outside of the wall of a single institution.

This specification is to be construed as illustrative only and is for the purpose of teaching those skilled in the art the manner of carrying out the disclosure. It is to be understood that the forms of the disclosure herein shown and described are to be taken as the presently preferred embodiments. As already stated, various changes may be made in the shape, size and arrangement of components of the system or device. Also adjustments made in the steps or order of the steps of the method without departing from the scope of this disclosure. For example, equivalent elements may be substituted for those illustrated and described herein and certain features of the disclosure may be utilized independently of the use of other features, all as would be apparent to one skilled in the art after having the benefit of this description of the disclosure.

While specific embodiments have been illustrated and described, numerous modifications are possible without departing from the spirit of the disclosure, and the scope of protection is only limited by the scope of the accompanying claims.

What we claim are:

1. A medical coding assignment and medical service reimbursement estimator system that receives clinician notations of patient symptoms during a patient encounter including simultaneously suggesting applicable codes, and displaying code suggestions to clinician along with projected or estimated third party reimbursement to assist in evaluating during the patient encounter the accuracy of the entered symptoms or any diagnosis, treatment or test plan comprising: a system that performs recording of patient symptoms or diagnosis, correlation to medical codes and estimating and displaying reimbursement information during the patient encounter including:
(a) an information input component to enter patient medical information including symptoms or diagnosis;
(b) a software program that correlates the patient medical information to a medical coding protocol and selects a medical code;
(c) an information communication component that communicates the medical code to a database of medical reimbursement correlated to medical codes; and
(d) a software program that correlates the communicated codes to medical reimbursement; and
(e) displays the medical code and correlated medical reimbursement.

2. The system of claim 1 further comprising correlation the patient symptoms or diagnosis to ICD, CPT or HCPCS code protocol.

3. The system of claim 1 further comprising correlation of the patient medical information to medical codes for diagnosis, treatment or testing.

4. The system of claim 1 further comprising a search of an EHR of the patient using the selected code and a display of the EHR search results.

5. A system allowing a clinician to enter medical information regarding a patient symptoms, a software subsystem to evaluate the medical information and correlate the medical information to a diagnosis, treatment or test code protocol, and display a correlated code to the clinician during a patient encounter for evaluation of possible diagnosis, test or treatment comprising:
(a) an information input component structured to enter medical information including patient symptoms during a patient encounter;
(b) a non-transitory memory component;
(c) a database of medical coding protocols wherein a numeric or alphanumeric code is listed with a code descriptor;
(d) a software program to access the database and evaluate and correlate the medical information to a medical code protocol pertaining to diagnosis, treatment or testing relevant to the patient medical information; and
(e) a display to disclose a correlated medical code during a patient encounter.

6. The medical coding assignment system of claim 5 further comprising:
(a) a communication component structured to access a network containing patient electronic health records;
(b) the software program structured to search and access the network for electronic health records of the patient utilizing the correlated code.

7. The medical coding assignment system of claim 6 further comprising the display of search results during the patient encounter.

8. The system of claim 7 further wherein the display is made during or before selection of diagnosis, treatment or testing relevant to the patient medical information.

9. A patient medical diagnosis, treatment or test evaluation method utilizing entry of patient symptoms into a computer software program and evaluation and correlation of the information by the software with a medical code protocol and displaying correlated codes during the patient encounter comprising:
(a) entering the patient symptom description into a device accessing a database of a medical code protocol;
(b) evaluating the patient symptom description with a medical code protocol and correlating a medical code to the patient symptom description;
(c) observing the correlated medical code;
(d) creating a patient diagnosis or patient treatment or test plan with the correlated medical code; and
(e) performing the steps (a) through (d) during a patient encounter.

10. The method of claim 9 further comprising:
(a) searching an EHR of the patient for instances of the correlated medical code or a related medical code; and
(b) evaluating the instances of the correlated medical code or related medical code within the search result; and
(c) including the evaluation in creating the patient diagnosis or patient treatment or test plan.

11. The method of claim 9 wherein the evaluation of patient symptom description with the code protocol includes creating a database of key medical terms from the code protocol and comparing contents of the patient symptom description with the database.

12. The method of claim 11 wherein the evaluation of patient symptom description with code protocol includes evaluation of synonyms.

13. The method of claim 9 further comprising the software utilizing machine learning to evaluate the patient symptom description with the code protocol.

14. The method of claim 13 wherein the machine learning includes creating a database of symptom descriptions of past patients and correlated codes.

15. The method of claim 13 wherein the machine learning includes evaluating symptom descriptions with medical literature or synonyms.

16. An method of a clinician creating a patient diagnosis, treatment or test plan by evaluating patient symptoms observed and evaluating diagnosis treatment and testing options during a patient encounter comprising: noting patient information of symptoms or possible diagnosis; entering the information of symptoms or diagnosis, and evaluating the information with a database accessed by software using machine learning and keyword matching; correlating the symptom and possible diagnosis information with relevant suggested diagnosis, treatment or test plans achieved from the evaluation of the medical code protocol; and displaying the suggested diagnosis, treatment and test plans to a clinician during a patient encounter.

17. The method of claim 16 further comprising initiating a search of the patient's medical history using a relevant suggested code from the medical code protocol for a medical diagnosis, treatment or test plan and displaying the search results to the clinician during the patient encounter.

18. Patient treatment and testing evaluation method comprising:
(a) initiating a patent encounter and entering information of patient symptom into a software program;
(b) evaluating the information with the software program wherein the software program contains or accesses in non-transitory memory a medical code protocol and correlating the information to the coding protocol;
(c) identifying a diagnostic code relevant to the information;
(d) identifying a test or treatment code utilizing the identified diagnostic code; and
(e) communicating the diagnostic code and the test or treatment code to a clinician during the patient encounter for evaluation of diagnosis and treatment or test plan.

19. The method of claim 18 wherein the evaluation of the information by the software program comprises matching of key words or machine learning wherein data of prior related symptom information correlated to medical codes is utilized in the evaluation.

20. The method of claim 19 wherein the clinician initiates a search of the patient medical records utilizing the identified diagnosis, treatment or test code and a result of the search is available for clinician evaluation of the diagnosis, treatment or test plan.
